(19) United States
(12) Patent Application Publication
(10) Pub. No.: US 2010/0166731 A1
(43) Pub. Date: Jul. 1, 2010

(54) Methods and Compositions for Treating Cancer

(76) Inventors: Steven R. Bartz, Seattle, WA (US); Peter Linsley, Encinitas, CA (US)

Correspondence Address: MERCK, P O BOX 2000, RAHWAY, NJ, 07065-0907, US

(21) Appl. No.: 11/992,798

(22) PCT Filed: Sep. 29, 2006
(86) PCT No.: PCT/US06/38350

§ 371 Date: Mar. 28, 2008

Related U.S. Application Data
(60) Provisional application No. 60/722,601, filed on Sep. 30, 2005.

(51) Int. Cl. A61K 39/395; A61K 33/24; C12Q 1/68; C12Q 1/48; C12N 5/02; A61K 38/00; A61P 35/04; A61K 31/7088

(52) U.S. Cl. 424/130.1; 424/649; 435/6; 435/15; 435/375; 514/2; 514/44 R; 514/44 A

(57) ABSTRACT

The present invention relates to methods and compositions for treating cancers by reducing the expression or activity of one or more of the genes encoding protein kinases ATR, MAST2, MAP3K6, TBK1, ADRBK2, CDKL2, LATS2, STK32B, STK11, DDR13 PSKH2, and NEK8, and/or the encoded kinases. The invention also relates to methods and compositions for determining the responsiveness of a cancer patient to anti-cancer drugs based on the status of one or more of such kinases. The invention further relates to methods and compositions for screening compounds that can be used to modulate the expression/activity of these kinases.

This application claims the benefit under 35 U.S.C. §119(e) of U.S. Provisional Patent Application No. 60/722,601, filed on Sep. 30, 2005, which is incorporated by reference herein in its entirety.

### 1. FIELD OF THE INVENTION

The present invention relates to methods and compositions for treating cancer by modulating the expression or activity of certain kinase genes and/or their encoded kinases. The invention also relates to methods and compositions for determining the responsiveness of a cancer patient to one or more anti-cancer drugs based on the status of such kinases. The invention further relates to methods and compositions for screening compounds that can be used to modulate the expression/activity of these kinases.

### 2. BACKGROUND OF THE INVENTION

Reversible protein phosphorylation is a predominant strategy for controlling the activity of proteins in eukaryotic cells (See, e.g., Molecular Biology of the Cell, third edition, Alberts et al., eds., Garland Publishing, Inc., New York, 1994). Covalent attachment of a phosphate group to an amino acid residue, such as a serine or tyrosine residue, in an amino acid side chain causes structural and conformational changes in a protein, which lead to changes in the activity of the protein, e.g., changes in the catalytic activity of the protein, changes in protein-protein interaction between the protein with its interaction partners, or changes in subcellular localizations of the protein. Such a protein activity control strategy is realized by two types of enzymes, protein kinases (PKs), which catalyze the addition of phosphates, and protein phosphatases (PPs), which catalyze the removal of phosphates. Eukaryotic cells contain a large variety of these enzymes for various important cellular processes, e.g., in cell signaling and cell cycle control.

Protein kinases can be divided into two major types based on their substrate specificity. One type of protein kinases is serine/threonine kinases (STKs) which catalyze the phosphorylation of serine or threonine residues. Another type of protein kinases is protein tyrosine kinases (PTKs) which catalyze the phosphorylation of tyrosine residues. Some kinases are dual specific, i.e., they are capable of phosphorylating both tyrosine and serine or threonine. The protein kinases may also be classified into several major groups including AGC, CAMK, Casein kinase 1, CMGC, STE, and tyrosine kinases (Plowman et al., 1999, Proc. Natl. Acad. Sci., USA, 96:13603-11610). Within each group the kinases can further be divided into different distinct families of more closely related kinases. In addition, there are a group designated “other” to represent several smaller families and a group designated “atypical” family to represent those protein kinases whose catalytic domain has little or no primary sequence homology to conventional kinases, including the alpha kinases, pyruvate dehydrogenase kinases, A6 kinases and PI3 kinases.

STKs include cyclic-nucleotide-dependent kinases, calcium/calmodulin kinases, cyclin-dependent kinases (CDKs), MAP-kinases, serine-threonine kinase receptors, and several other less defined subfamilies. STKs generally contain a homologous catalytic subunit and one or more regulatory subunits. PTKs can be divided into soluble (or non-receptor) PTKs and membrane-bound (receptor-like) PTKs. The non-receptor PTKs comprise one or more catalytic domains flanked by one or more non-catalytic domains. The receptor-like PTKs comprise one or more transmembrane domains and a receptor domain. The catalytic domain of a receptor PTK, which is exposed on the cytoplasmic side of the plasma membrane, is activated when an extracellular molecule binds to the extracellular receptor domain (See, e.g., Molecular Biology of the Cell, third edition, Alberts et al., eds., Garland Publishing, Inc., New York, 1994; and Pingel and Thomas, 1989, Cell 58:1055-1065).

Protein kinases play important roles in cell signaling pathways which control fundamental cellular processes including growth and differentiation, cell cycle progression, and cytoskeletal function. PKs are implicated in modulation of cytoskeletal integrity and related cellular phenomena such as transformation, tumor invasion, metastasis, cell adhesion, and leukocyte movement along and passage through the endothelial cell layer in inflammation. Due to their involvement in vital cellular processes, modulating the activity of protein kinases may have potential therapeutic effects as a way of modulating, e.g., cell signaling and cell growth and proliferation. Therefore, agents that modulate the activity of protein phosphatases may be important drug candidates for diseases such as cancer. For example, U.S. Patent Publication No. 2005012582 discloses nucleic acid and amino acid sequences of novel human kinases and their uses in the diagnosis and treatment of diseases.

Ataxia-Telangiectasia and Rad3-related protein (ATR), also known as FRAP-related protein 1 (FRP 1), is a member of the phosphotidylinositol kinase (PIK)-related kinase family, which is involved in cell cycle progression, DNA recombination, and detection of DNA damage. Initially cloned from a Jurkat T-cell cDNA library, the ATR open reading frame encompasses 7.9 kb, encoding a predicted 2,644 amino acid protein. This gene was also mapped to chromosome 3. ATR is most closely related to three other PIK-related kinase family members involved in checkpoint function, MEI41 (Drosophila), MEC1P (S. cerevisiae), and RAD3 (S. pombe), and may be the human homolog of these kinases (Cimprich et al., 1996, Proc. Natl. Acad. Sci. USA 93:2850-2855).

ATR is involved in the response to DNA damage induced by ionizing radiation and ultra-violet irradiation (Sarkaria et al., 1998, Cancer Res. 58:4375-4382; Wright et al., 1998, Proc. Natl. Acad. Sci. USA 95:7445-7450). Phosphorylation of RAD 17 checkpoint protein by ATR and ATM (Ataxia-Telangiectasia Mutated) is critical for DNA-damaged induced checkpoint response (Bao et al., 2001, Nature 411:969-974). Casper et al. (2002, Cell 111:779-789) demonstrated that ATR regulates fragile site stability. Common fragile sites are loci that exhibit gaps and breaks on metaphase chromosomes under conditions of replicative stress. These sites are hot spots for sister chromatid exchanges, translocations, and deletions.

ATRIP was identified as an ATR-interacting protein that is phosphorylated by ATR, regulates ATR expression, and is an essential component of the DNA damage checkpoint pathway. Both ATR and ATRIP co-localize to intranuclear foci after DNA damage or inhibition of replication. Deletion of or interference with ATR or ATRIP caused the loss of both ATR and ATRIP expression and the loss of checkpoint responses, suggesting that ATR and ATRIP are mutually dependent partners in cell cycle checkpoint pathways (Cortez et al., 2001, Science, 294:1713-1716). Zhou and Elledge (2003, Science 300:1542-1548) demonstrated that the ATR-ATRIP kinase complex is crucial for cellular response to replication stress and DNA damage through its interaction with replication protein A (RPA) complex, which associates with single-stranded DNA. The binding of ATRIP to RPA-coated single-stranded DNA allows the ATR-ATRIP complex to phosphorylate the RAD17 checkpoint protein.

Alternative splice variants of ATR have been identified in multiple human tissues (Mannino et al., 2001, Gene 272:35-43). O'Driscoll et al. (2003, Nat. Genet. 33:497-501) identified a synonymous mutation affecting ATR splicing of exon 9 and results in Seckel syndrome, characterized by microcephaly, dwarfism, and mental retardation. Patients homozygous for the mutation have low levels of correctly spliced ATR. Casper et al. (2004, Am. J. Hum. Genet. 75:654-660) demonstrated that patients carrying this Seckel Syndrome mutation in ATR showed increased chromosomal instability, particularly at fragile sites, after treatment with aphidicolin, a DNA polymerase inhibitor, compared with controls.

ATR deficiency results in early embryonic lethality in mice and cells displayed to genome disruption, suggesting the essential role of this protein in cell cycle and genome integrity (Brown and Baltimore, 2000, Genes and Dev. 14:397-402; de Klein et al., 2000, Curr. Biol. 10:479-482). Cha and Kleckner (2002, Science 297:602-606) found that elimination of MEC1 function in yeast, the homolog of mammalian ATR, results in genome-wide fork stalling followed by chromosomal breakage.

Multiple studies have investigated the role of ATR in the sensitivity of cancer cells to chemotherapy. ATR knockdown by RNA interference in p53-defective PC3 prostate cancer cells increased their sensitivity to doxorubicin compared to normal cells (Mukhopadhyay et al., 2005, Cancer Res. 65:2872-2881). Flatten et al. (2005, J. Biol. Chem. 280:14349-14355) also showed that ATR siRNA-induced sensitization to topoisomerase I poison occurs in cultured cells with either an active or inactive p53 pathway. Prostate cancer-derived DU145 cells demonstrated enhanced sensitivity to alkylating agent methyl methanesulfonate (MMS) following ATR RNA interference (Collis et al., 2003, Cancer Res. 63:1550-1554).

ATR polynucleotides, expression vector, and methods of making ATR polypeptides have been claimed (U.S. Pat. No. 6,632,936). Methods of screening for anti-cancer therapies using ATR have also been described (US 2003/0007975). Methods for screening for agents that modify ATR function or ATR-ATRIP interaction have been described (WO 2003/044214). ATR has been disclosed as one of a set of gene biomarkers for determining the response of a mammal to a cancer treatment comprising administration of a modulator of cyclin-dependent kinase activity (WO 2005/012875). WO 2004043406 discloses siRNA probes useful for knocking down ATR function and sensitizing cancer cells to DNA damaging agents.

Microtubule associated serine/threonine kinase 2 (MAST2), also known as MAST205, was initially identified in a search for proteins that associate with microtubules during spermatogenesis. MAST2 was found to co-localize with the microtubular machete of developing spermatids and may provide a link between the signal transduction pathway, microtubule organization, and sperm head shaping. Sequencing revealed that MAST205 is a novel serine/threonine kinase with a catalytic domain related to those of the A and C kinase families. The microtubule binding-domain occupies the central region of the molecule, including the kinase domain, and a portion of the C-terminus. MAST205 expression was found to be regulated during testicular development, increasing in abundance during prepuberal testicular development (Walden et al., 1993, Mol. Cell. Biol. 13:7625-7635).

MAST2 was found to interact with the protocadherein LKC, whose function is implicated in contact inhibition of cell proliferation (Okazaki et al., 2002, Carcinogenesis 23:1139-1148). Lumeng et al. (1999, Nat. Neurosci. 2:611-617) reported that MAST2 interacts with cortical microtubule filaments through the formation of a β2-syntrophin-dystrophin/utrophin complex, which is found at neuromuscular junctions and neuronal postsynaptic densities.

MAST2 has also been implicated in the LPS signal transduction pathway, leading to activation of NF-κB. RNA interference of MAST2 resulted in inhibition of LPS-stimulated IL-12 promoter activity and IL-12 secretion in macrophages. Similarly, a dominant negative MAST2 mutant blocked IL-12 synthesis and NF-κB activation following LPS stimulation. Additionally, MAST2 is rapidly ubiquinated and degraded following macrophage FcγR ligation (Zhou et al., 2004, J. Immunol. 172:2559-2568). Xiong et al. (J. Biol. Chem. 2004, 279:43675-43683) demonstrated that MAST2 forms a complex with TRAF6, an E3 ubiquitin ligase, resulting in the inhibition of TRAF6 NF-κB activation.

MAST2 expression was also found to be up-regulated in the muscle of diabetic patients vs. lean non-diabetic individuals, and the MAST2 expression was down-regulated in the muscle of diabetic patients after troglitazone treatment (WO 2003/103601). MAST2 was also identified as a modifier of beta-catenin (MBCAT) using a genetic screen in C. elegans. Uses of MBCATs for identifying candidate therapeutic agents for treatment of disorders associated with defective or impaired beta-catenin and/or MBCAT function, such as an angiogenic, apoptotic or cell proliferation disorder, have been suggested (US 2003/224406). MAST2 sequence and methods of screening for modulators have also been disclosed in WO 2004/006838.

Mitogen-activated protein kinase kinase kinase 6 (MAP3K6), also known as MPKKK6, was identified via a yeast two-hybrid screen used to find proteins that bind to MAP3K5/ASK1, which activates c-Jun N-terminal kinase (JNK) and p38 kinase signaling pathways and induces apoptosis when expressed in stably transfected cells. MAP3K6 encodes a predicted 1,280 amino acid protein, which shares 45% identity with MAP3K5. MAP3K6 transcripts were observed in human heart and skeletal muscle, and weaker signals were detected lung, liver, kidney, testis and spleen. Recombinant MAP3K6 only weakly activated the JNK pathway but not the p38 or ERK pathways in transfected cells (Wang et al., 1998, Biochem. Biophys. Res. Commun. 253:33-37).

MAP3K6 was identified as one of a group of genes differentially expressed in gastric cancer (WO 2003/059148).

Phosphorylation of IκB by the IκB kinase (IKK) complex marks IκB for degradation, resulting in activation of NF-κB. The IKK complex consists of two catalytic subunits, IKKα and IKKβ, with the IKKβ subunit being required for NF-κB activation by proinflammatory cytokines. A tank-binding kinase 1 (TBK1), also known as NF-κB-Activating Kinase (NAK), which has kinase activity on IKKβ, inducing IKB degradation and NF-κβ activity, was amplified from a fetal brain cDNA library using degenerate primers based sequences common to IKKα and IKKβ. (Tojima et al., 2000, Nature 404:778-782). Like IKKα/β, TBK1 contains leucine-zipper and helix-loop-helix motifs in its carboxy region. However, while IKKα and IKKβ contain two serines in their respective activation loops, TBK1 substitutes glutamic acid (Glu168) for one of these serines. A 2.2 kb TBK1 transcript was ubiquitously detected by Northern blot, with highest expression in the testis. Functional analysis demonstrated that TBK1 can phosphorylate only one of the two regulatory serines of IκB, but can phosphorylate both serines in the IKKβ and stimulate its IκB kinase activity (Tojima et al., 2000, Nature 404:778-782).

In addition to its function in NF-κB activation, TBK1 function has also been found to have a role in activation of the IRF3 signaling pathway, triggering host antiviral response to viral infection (Sharma et al., 2003, Science 300:1148-1151; Fitzgerald et al., 2003, Nat. Immun. 491-496).

Pomerantz and Baltimore (1999, EMBO J. 18:6694-6704) demonstrated that TBK1 mediates TANK protein's ability to activate NF-κB. TBK1 functions in a TBK1-TANK-TRAF2 signaling complex upstream of NIK and the IKK complex. Pomerantz and Baltimore also cloned the mouse TBK1, which is 94% identical to human TBK1. The human TBK1 gene contains 21 exons and is located on chromosome 13 (Li et al., 2003, Gene 304:57-64).

TBK1^{-/-} mice die at embryonic day 14.5 of apoptotic liver degeneration and show impaired NF-κB-dependent gene transcription (Bonnard et al., 2000, EMBO J. 19:4976-7985). Study of embryonic fibroblasts from TBK1^{-/-} mice showed that TBK1 is required for activation and nuclear translocation of IRF3 in mouse embryonic fibroblasts (McWhirter et al., 2004, Proc. Natl. Acad. Sci. USA 101:233-238). These cells showed marked defects in expression of IFN-α, IFN-β, IP-10, and RANTES gene expression after Sendai or Newcastle disease viral infection, suggesting that TBK1 is important for IRF3-dependent antiviral gene expression.

TBK1 polynucleotides, protein, expression vectors, and screening methods for identifying TBK modulators have been disclosed (WO 0144444; U.S. Pat. No. 5,837,514; U.S. Pat. No. 5,776,717). WO 2005/035746 relates to the use of TBK1 in methods of screening and treatment of diseases marked by abnormal angiogenesis. TBK1 was also identified as a member of a polypeptide complex which includes TNF-α and/or TNF-α receptor and demonstrate anti-inflammatory properties and cytostatic activities and may be useful for screening for modulators of apoptosis and inflammation (WO 2004/012673).

β-adrenergic receptor kinase (βARK) is a serine/threonine kinase that phosphorylates the agonist-occupied form of the β-adrenergic and related G-protein coupled receptors. Benovic et al. (1991, J. Biol. Chem. 266:14939-14946) identified a second beta-adrenergic receptor kinase (ADRBK2), also known as βARK2 or GRK3, by screening a bovine brain cDNA library using a catalytic domain fragment of the βARK cDNA. Bovine ADRBK2 has 85% identity with ADRBK1. In the rat, ADRBK2 transcripts were detected primarily in neuronal tissues, though low levels were also observed in peripheral tissues. Parruti et al. (1993, Biochem. Biophys. Res. Commun. 190:475-481) cloned ADRBK2 from a human pituitary cDNA library using the catalytic domain of ADRBK1. The predicted human ADRBK2 protein consisted of 688 amino acids which shares 84% identity with ADRBK1 and 95% identity with the bovine ADRBK2. Northern blot analysis revealed ADRBK2 transcripts in monocytes, granulocytes, and a neuroblastoma cell line, as well as in lung, heart, and adipose tissue.

Human ADRBK2 was mapped to chromosome 22 (Calabrese et al., 1994, Genomics 23:286-288) while the mouse ADRBK2 gene was mapped to chromosome 5 (Benovic et al., 1991, J. Biol. Chem. 266:14939-14946).

Barrett et al. (2003, Mol Psych. 8:546-557) suggested that a single nucleotide polymorphism in the promoter region of ADRBK2 is associated with bipolar disorder. Dzimiri et al. (2004, Eur. J. Pharmacol. 489:167-177) detected differential expression of ADRBK2 in the right ventricle of the volume overload patients. ADBRK2 has also been to implicated in the desensitization of μ-opiod receptors (Celver et al., 2001, J. Biol. Chem. 276:4894-4900; Mandyam et al., 2002, J. Pharmacol. Exp. Ther. 302:502-509).

WO 2003/097795 describes a method for identifying a compound that alters GPR internalization, including ADRBK2, which may be useful for treating disorders associated with aberrant GRP desensitization. ADRBK2 has also been described as a member protein of complex protein-protein interactions in adipocyte cells, which may be used for identifying compounds that modulate the protein-protein interactions for the treatment of obesity and metabolic disorders (WO 2002/53726).

The 56 kDa cyclin-dependent kinase-like 2 (CDKL2) gene, also known as p56 or KKIAMRE, was molecularly cloned from human fetal brain (Taglienti et al., 1996, Oncogene 13:2563-2574). The predicted 493 amino acid CDKL2 protein is related to the proline-directed protein kinase group of signal transducing enzymes and has 58% identity with p42 KKIALRE in the kinase domain. CDKL2 and KKIALRE displayed mutually exclusive expression in the reproductive tissues, where CDKL2 was expressed in the testis and KKIALRE was expressed in the ovary. Northern blot analysis showed two major CDKL2 transcripts in the adult testis and kidney, and lower levels in the adult brain and lung. A single transcript was found in fetal brain and fetal kidney. CDKL2 was activated by treatment of cultured cells with epidermal growth factor (EGF). However, EGF activation of CDKL2 did not require phosphorylation of the conserved MAP kinase dual phosphorylation motif, suggesting that CDKL2 may not be a functional member of the MAP kinase family (Taglienti et al., 1996, Oncogene 13:2563-2574).

The sequence, gene structure, expression pattern, and cDNA diversity of the mouse CDKL2 gene has also been investigated. The mouse CDKL2 gene consists of 15 exons, and multiple variants have identified, generated by alternative splicing in the carboxyl-terminal regulatory region as well as the 5′ noncoding region. In situ hybridization and immunohistochemistry detected CDKL2 expression in neurons of various brain regions, including the cerebral cortex, thalamus, hippocampus, olfactory bulb, and deep cerebellar nuclei. Transcripts were also detected in the mouse lung and kidney by Northern blot (Sassa et al., 2000, J. Neurochem. 74:1809-1819). Sassa et al. (2004, Cell. Tissue Res. 315:147-156) studied postnatal expression of CDKL2 in the mouse brain, using mutant mice in which the LACZ gene is expressed under the control of CDKL2 promoter/enhancer. LACZ expression was first detected in the mouse cerebral cortex at postnatal days 3-7, and increased gradually to near maximum at day 28.

CDKL2 expression was found to increase in deep cerebellar nuclei of rabbits after eyeblink conditioning, a model of learning and memory. CDKL2 expression in other rabbit tissues was consistent with findings in mouse and human (Gomi et al., 1999, J. Neurosci. 19:9530-9537). The rabbit CDKL2 polypeptide, associated vector and host cell, and method of producing the polypeptide have been claimed (U.S. Pat. No. 6,664,086; U.S. Pat. No. 6,428,994). CDKL2 was also described as useful for diagnosis and screening methods related to pain disorders (WO 2003/073983).

Large Tumor Suppressor, homolog 2 (LATS2), also known as KPM, is one of two human homologs of Drosophila LATS/WARTS (the other being LATS 1), which encode putative serine/threonine kinases. LATS2 maps to chromosome 13q11-12, a known hot-spot for loss-of-heterozygosity (LOH) in non-small cell lung cancer. LATS2 inhibits the G1/S cell-cycle transition, and ectopic expression suppresses tumor growth in nude mice (Li et al., 2003, Oncogene 22:43498-4405). LATS2 negatively interacts with the androgen receptor

(AR) and inhibits androgen-regulated gene expression, suggesting a role in prostate cancer (Powzaniuk et al., 2004, Mol. Endocrinol. 18:2011-2023). Ectopically expressed LATS2 also induces apoptosis by downregulating the anti-apoptotic proteins BCL-2 and BCL-X(L) (Ke et al., 2004, Exp. Cell Res. 15:329-339). LATS2 deficiency in mice results in embryonic lethality on or before day 12.5, which is accompanied by overgrowth in restricted tissues of mesodermal lineage. LATS2^{-/-} mouse embryonic fibroblasts acquired a growth advantage and exhibited centrosome amplification and defective cytokinesis, consistent with the localization of LATS2 protein to the centrosome, suggesting that LATS2 has a role in maintenance of mitotic fidelity and genomic integrity (MacPherson et al., 2004. EMBO J. 23:3677-3688).

The human LATS2 gene maps to chromosome 13, consists of 7 coding exons, and encodes a predicted protein of 1,088 amino acids with a C-terminal serine/threonine kinase domain. LATS2 is most closely related to the mouse and human LATS1 proteins, followed by Drosophila LATS. Endogenous LATS2 is a nuclear protein of ˜125 kd. Northern analysis detected a 5.8 kb transcript in several tissues, with highest expression in heart and skeletal muscle. The testis expressed a 3.8 kb transcript (Yabuta et al., 2000, Genomics 63:263-270).

LATS2 nucleotide sequences, expression vectors for producing LATS2 polypeptides and fusion proteins, and methods of identifying compounds that modulate the function of LATS2 in cells have been claimed (U.S. Pat. No. 6,495,353).

Human serine/threonine protein kinase 32B (STK32B), is also known as STKG6, YANK2, STK32, and HSA250839. STK32B is linked to the EVC locus, which is implicated in Ellis-van Creveld syndrome; however, the two genes are distinct (Ruiz-Perez et al., 2000, Nature Genetics 24:283-286).

STK32B nucleotide sequences are disclosed (US 2005/0054826; US 2004/0038337).

Human serine/threonine protein kinase 11 (STK11), also known as PJS (Peutz-Jeghers syndrome) and LKB1, regulates chromatin remodeling, cell-cycle arrest, WNT signaling, cell polarity, and energy metabolism, and functions as a tumor suppressor (see review by Marignani, P., 2005, J. Clin. Pathol. 58:15-19). STK11 is homologous to the Xenopus laevis embryonically expressed kinase XEEK1 (Su et al., 1996, J. Biol. Chem. 271:14430-14437). Mutations in STK11 are associated with Peutz-Jeghers syndrome, an autosomal dominant disorder characterized by growth of polyps in the gastrointestinal tract, pigmented macules on the skin and mouth, and other neoplasms. Most patients with Peutz-Jeghers syndrome show germline mutations in STK11, with a minority having somatic mutations. The cytoplasmic-nuclear localization of the protein appears to play a role in pathogenicity, such that mutations that interfere with cytoplasmic retention are associated with PJS (Nezu et al., 1999, Biochem. Biophys. Res. Comm. 261:750-755).

STK11 physically associates with P53 and regulates P53 dependent apoptosis pathways, and STK11 is absent from intestinal polyps, suggesting that deficiency in apoptosis plays a role in polyp formation (Karuman et al., 2001, Mol. Cell 7:1307-1319). A yeast 2-hybrid system identified a leucine-rich repeat protein called LIP1 that interacts with STK11, and may regulate the cytoplasmic localization of STK11. LIP1 also interacts with SMAD4, forming STK11-LIP1-SMAD4 ternary complexes. SMAD4 mutations are associated with juvenile intestinal polyposis syndrome (PJI), suggesting a link between PJS and PJI (Smith et al., 2001, Hum. Mol. Genet. 8:1479-1485). Ectopic expression of STK11 in cancer cell lines defective for STK11 expression resulted in G1 cell cycle arrest. Kinase-defective Peutz-Jeghers syndrome mutants of STK11 localized predominantly to the nucleus. Moreover, when STK11 was forced to remain cytoplasmic through disruption of the nuclear localization signal, it retained full growth suppression activity in a kinase-dependent manner. STK11 expression also up-regulated P21 promoter activity in a P53 dependent manner (Tiainen et al., 2002, Hum. Mol. Genet. 11:1497-1504). Micro-array experiments suggest that mutant STK11 proteins fail to activate GSK-3beta kinase, which normally inhibits the WNT pathway, suggesting that activation of WNT signaling may contribute to the cancer predisposition of Peutz-Jeghers syndrome patients (Lin-Marq et al., 2005, Mol. Genet. Genomics 273:184-196). STK11 is activated by the pseudokinase Ste20-Related-Adaptor (STRAD) protein, which forms a complex with STK11 and results in phosphorylation of both proteins. STRAD translocates wild-type, but not mutant forms of STK11 from the nucleus to the cytoplasm. Removal of endogenous STRAD by siRNA abolished the STK11 induced G1 cell cycle arrest, whereas mutant forms of STK11 that do not interact with STRAD also fail to induce G1 arrest, suggesting that STRAD plays a role in regulating the tumor suppressor functions of STK11 (Bass et al., 2003, EMBO J. 22:3062-3072).

Several groups have generated mice with mutations in the STK11 gene. Homozygous STK11^{-/-} mice die in utero between 8.5 and 9.5 days, due in part to defective vasculogenesis associated with a tissue-specific deregulation of vascular endothelial growth factor (VEGF) (Ylikorkala et al., 2001, Science 293: 1323-1326). Heterozygous mice develop gastric and intestinal polyps histologically similar to those in Peutz-Jeghers syndrome. The wild-type allele was not mutated, suggesting that the initiation of polyposis is not due to loss of heterozygosity in STK11 (see Ylikorkala et al., 2001, Science 293: 1323-1326; Miyoshi et al., 2002, Cancer Res. 62:2261-2266; Jishage et al., 2002, PNAS 99:8903-8908; and Bardeesy et al., 2002, Nature 419: 162-167). Moreover, STK11 was shown to be haploinsufficient for tumor suppression in heterozygous mice (Rossi et al., 2002, PNAS 99:12327-12332). This same study demonstrated that cyclooxygenase-2 (COX2) protein was highly up-regulated in polyps from heterozygous STK11 mice, along with the activated forms of extracellular signal-regulated kinases 1 and 2 (ERK1/2), and that most human polyps examined also up-regulated COX2, thus identifying COX2 as a potential target for chemoprevention (Rossi et al., 2002, PNAS 99:12327-12332).

STK11 mRNA is ubiquitously expressed in humans, with generally higher levels in fetal tissues and lower levels in adult tissues, except for the testis which showed relatively high adult expression. STK11 expression was largely confined to epithelia, which is consistent with the epithelial origin of most cancers in Peutz-Jeghers syndrome (Rowan et al., 2000, J. Pathol. 192:203-206). In the mouse, STK11 mRNA is also ubiquitously expressed during early embryogenesis, becoming more restricted at later stages with high expression levels observed in testis (Luukko et al., 1999, Mech. Dev. 83:187-190). STK11 is located on chromosome 19p13.3 and the gene is composed of 9 coding exons (Schumacher et al., 2005, J. Med. Genet. 42:428-435).

STK11 sequences are disclosed (U.S. Pat. No. 5827726; U.S. Pat. No. 6,800,436; U.S. Pat. No. 6,500,938) and methods of use described (U.S. Pat. No. 6,800,436; U.S. Pat. No. 6,500,938). STK11 gene knockout mice are disclosed (U.S. Pat. No. 6,791,006).

Discoidin domain receptor family, member 1 (DDR1), also known as CAK, DDR, NEP, PTK3, PTK3A, RTK6, TRKE, CD167, EDDR1, MCK10, and NTRK4, is a receptor tyrosine kinase (RTK). DDR1 belongs to a subfamily of RTK's with homology to the Dictyostelium discoideum protein discoidin I in the extracellular domain, a single transmembrane domain, an extended juxtamembrane region, and a catalytic tyrosine kinase domain (Vogel, W., 1999, FASEB J. 13 (Suppl.):S77-S82). DDR1 is activated by all collagens so far tested (type I to type V), which is consistent with a function in cell adhesion to the extracellular matrix (Vogel, W., 1999, FASEB J. 13 (Suppl.):S77-S82). DDR1 mRNA expression is restricted to epithelial cells, particularly in the kidney, lung, gastrointestinal tract, and brain, (Alves et al., 1995, Oncogene 10:609-618). DDR1 is significantly overexpressed in several human tumors from breast, ovarian, esophageal, and pediatric brain, (Vogel, W., 1999, FASEB J. 13 (Suppl.):S77-582), and DDR1 is expressed in highly invasive tumor cells (Alves et al., 1995, Oncogene 10:609-618). The DDR1 promoter contains a consensus binding site for P53, and expression of DDR1 is upregulated by P53 in human osteosarcoma cells (Sakuma et al., 1996, FEBS Lett. 398:165-169). The activation of DDR1 requires WNT-5A-mediated stimulation of SRC non-receptor tyrosine kinases (Dejmek et al., 2003, Int. J. Cancer 103:344-351).

DDR1 also plays a role in leukocyte activation by collagen. DDR1 is expressed on human leukocytes, including neutrophils, monocytes, and lymphocytes in vitro (Yoshimura et al., 2005, Immunol. Res. 31:219-230). Activation of DDR1 on CD14+ cells from patients with idiopathic pulmonary fibrosis induced the production of chemokines and matrix metalloproteinase-9 (MMP9), whereas DDR1 activation of CD14+ cells from control patients did not induce chemokine or MMP-9 production (Matsuyama et al., 2005, J. Immunol. 174:6490-6498).

The DDR1 gene is located on chromosome 6p21.3, is encoded by 15 exons spanning ˜9 Kb, and there are three isoforms generated by alternative splicing (NM_{—}001954, NM_{—}013994, NM_{—}013993) (Sakuma et al., 1996, FEBS Lett. 398:165-169).

DDR1^{-/-} knockout mice are viable but significantly smaller than littermates, and female DDR1^{-/-} mice show defects in blastocyst implantation and mammary gland development (Vogel et al., 2001, Mol. Cell. Biol. 21:2906-2917). Expression of DDR1 mRNA and protein increased after balloon catheter injury of the rat carotid artery. In DDR1 knockout mice, the neointima area and the amount of collagen deposited were significantly lower following mechanical injury of the carotid arteries (Hou et al., 2001, J. Clin. Invest. 107:727-735). Further, DDR1^{-/-} mice have defects in kidney function associated with a disrupted glomerular basement membrane (Gross et al., 2004, Kidney Int'l 66:102-111).

DDR1 nucleotide sequences are disclosed (U.S. Pat. No. 6,627,733; U.S. Pat. No. 5,677,144; U.S. Pat. No. 6,607,879; U.S. Pat. No. 5,709,858 and related U.S. Pat. No. 6,001,621, U.S. Pat. No. 6,087,144, U.S. Pat. No. 6,096,527, U.S. Pat. No. 6,825,324) and methods of use described (U.S. Pat. No. 6,607,879).

Protein serine kinase H2 (PSKH2) is a serine/threonine kinase. PSKH2 sequences are disclosed (US 2004/0033493).

Never In Mitosis, Gene A-Related Kinase 8 (NEK8), also known as NEK12A, is a homolog of the filamentous fungus Aspergillus nidulans gene Never In Mitosis, gene A (NIMA) which controls mitotic signaling. It is closely related to murine NEK8 and human NEK9 (previously called NEK8, see Holland et al., 2002, J. Biol Chem 277:16229). There are currently 11 members of the human NEK serine/threonine protein kinase family, which share homology with the amino-terminal kinase domain of NIMA but diverge in their carboxy-terminal domains. NIMA is required for the G2-M cell cycle transition, as is human NEK2. Expression levels of endogenous NEK8 RNA are very low; by semi-quantitative PCR, NEK8 was detected in thyroid, adrenal gland, and skin tissues, and at lower levels in spleen, colon, and uterus. However, NEK8 mRNA is overexpressed in a variety of primary breast tumors. Overexpression of a kinase domain mutant form of NEK8 protein moderately increased CDK1/CyclinB1 protein and reduced actin protein levels, suggesting that NEK8 may be involved in cell cycle progression (Bowers and Boylan, 2004, Gene 328:135-142).

The murine NEK8 gene is mutated in the jck (juvenile cystic kidney) murine model of autosomal polycystic disease (ARPKD) (Liu et al., 2002, Development 129:5839-5846). A proteomic analysis of homozygous jck mice revealed galectin-1, sorcin and vimentin were induced in the kidneys, and increased accumulation and phosphorylation of the major urinary proteins (MUP) was also observed (Valkova et al., 2005, Mol Cell Proteomics, 4:1009-1018).

The human NEK8 gene is located on chromosome 17q11.1. The open reading frame of NEK8 encodes a 692 amino-acid protein with a calculated molecular weight of 75 kd (Bowers and Boylan, 2004, Gene 328:135-142).

NEK8 sequences are disclosed (U.S. Pat. No. 6,815,188; U.S. Pat. No. 6,593,125; U.S. Pat. No. 6,783,969).

RNA interference (RNAi) is a potent method to suppress gene expression in mammalian cells, and has generated much excitement in the scientific community (Couzin, 2002, Science 298:2296-2297; McManus et al., 2002, Nat. Rev. Genet. 3, 737-747; Hannon, G. J., 2002, Nature 418, 244-251; Paddison et al., 2002, Cancer Cell 2, 17-23). RNA interference is conserved throughout evolution, from C. elegans to humans, and is believed to function in protecting cells from invasion by RNA viruses. When a cell is infected by a dsRNA virus, the dsRNA is recognized and targeted for cleavage by an RNaseIII-type enzyme termed Dicer. The Dicer enzyme “dices” the RNA into short duplexes of 21nt, termed siRNAs or short-interfering RNAs, composed of 19nt of perfectly paired ribonucleotides with two unpaired nucleotides on the 3′ end of each strand. These short duplexes associate with a multiprotein complex termed RISC, and direct this complex to mRNA transcripts with sequence similarity to the siRNA. As a result, nucleases present in the RISC complex cleave the mRNA transcript, thereby abolishing expression of the gene product. In the case of viral infection, this mechanism would result in destruction of viral transcripts, thus preventing viral synthesis. Since the siRNAs are double-stranded, either strand has the potential to associate with RISC and direct silencing of transcripts with sequence similarity.

It has also been shown that siRNA and shRNA can be used to silence genes in vivo. The ability to utilize siRNA and shRNA for gene silencing in vivo has the potential to enable selection and development of siRNAs for therapeutic use. A recent report highlights the potential therapeutic application of siRNAs. Fas-mediated apoptosis is implicated in a broad spectrum of liver diseases, where lives could be saved by inhibiting apoptotic death of to hepatocytes. Song (Song et al. 2003, Nat. Medicine 9, 347-351) injected mice intravenously with siRNA targeted to the Fas receptor. The Fas gene was silenced in mouse hepatocytes at the mRNA and protein levels, prevented apoptosis, and protected the mice from hepatitis-induced liver damage. Thus, silencing Fas expression holds therapeutic promise to prevent liver injury by protecting hepatocytes from cytotoxicity. As another example, mice were injected intraperitoneally with siRNA targeting TNF-α. Lipopolysaccharide-induced TNF-α gene expression was inhibited, and these mice were protected from sepsis. Collectively, these results suggest that siRNAs can function in vivo, and may hold potential as therapeutic drugs (Sorensen et al., 2003, J. Mol. Biol. 327, 761-766).

Martinez et al. reported that RNA interference can be used to selectively target oncogenic mutations (Martinez et al., 2002, Proc. Natl. Acad. Sci. USA 99:14849-14854). In this report, an siRNA that targets the region of the R248W mutant of p53 containing the point mutation was shown to silence the expression of the mutant p53 but not the wild-type p53.

Wilda et al. reported that an siRNA targeting the M-BCR/ABL fusion mRNA can be used to deplete the M-BCR/ABL mRNA and the M-BRC/ABL oncoprotein in leukemic cells (Wilda et al., 2002, Oncogene 21:5716-5724). However, the report also showed that applying the siRNA in combination with Imatinib, a small-molecule ABL kinase tyrosine inhibitor, to leukemic cells did not further increase in the induction of apoptosis.

U.S. Pat. No. 6,506,559 discloses a RNA interference process for inhibiting expression of a target gene in a cell. The process comprises introducing partially or fully doubled-stranded RNA having a sequence in the duplex region that is identical to a sequence in the target gene into the cell or into the extracellular environment. RNA sequences with insertions, deletions, and single point mutations relative to the target sequence are also found as effective for inhibition of expression.

U.S. Patent Application Publication No. US 2002/0086356 discloses RNA interference in a Drosophila in vitro system using RNA segments 21-23 nucleotides (nt) in length. The patent application publication teaches that when these 21-23 nt fragments are purified and added back to Drosophila extracts, they mediate sequence-specific RNA interference in the absence of long dsRNA. The patent application publication also teaches that chemically synthesized oligonucleotides of the same or similar nature can also be used to target specific mRNAs for degradation in mammalian cells.

PCT publication WO 02/44321 discloses that double-stranded RNA (dsRNA) 19-23 nt in length induces sequence-specific post-transcriptional gene silencing in a Drosophila in vitro system. The PCT publication teaches that short interfering RNAs (siRNAs) generated by an RNase III-like processing reaction from long dsRNA or chemically synthesized siRNA duplexes with overhanging 3′ ends mediate efficient target RNA cleavage in the lysate, and the cleavage site is located near the center of the region spanned by the guiding siRNA. The PCT publication also provides evidence that the direction of dsRNA processing determines whether sense or antisense target RNA can be cleaved by the produced siRNP complex.

U.S. Patent Application Publication No. US 2002/016216 discloses a method for attenuating expression of a target gene in cultured cells by introducing double stranded RNA (dsRNA) that comprises a nucleotide sequence that hybridizes under stringent conditions to a nucleotide sequence of the target gene into the cells in an amount sufficient to attenuate expression of the target gene.

PCT publication WO 03/006477 discloses engineered RNA precursors that when expressed in a cell are processed by the cell to produce small interfering RNAs (siRNAs) that selectively silence targeted genes (by cleaving specific mRNAs) using the cell's own RNA interference (RNAi) pathway. The PCT publication teaches that by introducing nucleic acid molecules that encode these engineered RNA precursors into cells in vivo with appropriate regulatory sequences, expression of the engineered RNA precursors can be selectively controlled both temporally and spatially, i.e., at particular times and/or in particular tissues, organs, or cells.

Discussion or citation of a reference herein shall not be construed as an admission that such reference is prior art to the present invention.

### 3. SUMMARY OF THE INVENTION

The invention provides a method for treating a mammal having a cancer, comprising administering to said mammal a therapeutically effective amount of a first agent, said first agent reducing the expression of a gene encoding a protein kinase selected from the group consisting of ATR, MAST2, MAP3K6, TBK1, ADRBK2, CDKL2, LATS2, STK32B, STK11, DDR1, PSKH2, and NEK8 and/or activity of said protein kinase, wherein said mammal is subject to a therapy comprising administering to said mammal a therapeutically effective amount of a composition comprising one or more anti-cancer agents different from said first agent. In a particular embodiment, method comprises (a) administering to said mammal a therapeutically effective amount of a first agent, said first agent reducing the expression of a gene encoding a protein kinase selected from the group consisting of ATR, MAST2, MAP3K6, TBK1, ADRBK2, CDKL2, LATS2, STK32B, STK11, DDR1, PSKH2, and NEK8 and/or activity of said protein kinase; and (b) administering to said mammal a therapeutically effective amount of a composition comprising one or more anti-cancer agents.

In one embodiment, said first agent comprises a substance selected from the group consisting of siRNA, antisense nucleic acid, ribozyme, and triple helix forming nucleic acid, each being capable of reducing the expression of said gene in cells of said cancer. In a specific embodiment, said first agent comprises an siRNA targeting said gene. In another specific embodiment, said first agent comprises 2, 3, 4, 5, 6, or 10 different siRNAs targeting said gene.

In another embodiment, said first agent comprises a substance selected from the group consisting of antibody, peptide, and small molecule, each being capable of reducing the activity of said protein kinase in cells of said cancer.

In another embodiment, said one or more anti-cancer agents are selected from the group consisting of topoisomerase I inhibitor, topoisomerase II inhibitor, DNA binding agent, anti-metabolite, anti-mitotic agent, and ionizing radiation. In a specific embodiment, said one or more anti-cancer agents are selected from the group consisting of camptothecin, cisplatin, gemcitabine, hydoxyurea, bleomycin, L-001000962-000Y, and 5-fluorouracil.

In one embodiment, said first agent and/or said anti-cancer agent is purified.

In another aspect, the invention provides a method for evaluating sensitivity of a cell to the growth inhibitory effect of an anti-cancer agent, said method comprising determining a transcript level of one or more genes each encoding a different protein kinase selected from the group consisting of ATR, MAST2, MAP3K6, TBK1, ADRBK2, CDKL2, LATS2, STK32B, STK11, DDR1, PSKH2, and NEK8 in said cell, and determining whether said transcript level is below a predetermined threshold level, wherein said transcript level below said predetermined threshold level indicates that said cell is sensitive to the growth inhibitory effect of said anti-cancer agent. In one embodiment, said cell is an ex vivo cell. In another embodiment, said cell is an in vivo cell.

In one embodiment, said agent is an anti-cancer agent selected from the group consisting of topoisomerase I inhibitor, topoisomerase II inhibitor, DNA binding agent, anti-metabolite, anti-mitotic agent, and ionizing radiation. In a specific embodiment, said anti-cancer agent is selected from the group consisting of camptothecin, cisplatin, gemcitabine, hydoxyurea, bleomycin, L-001000962-000Y, and 5-fluorouracil.

In one embodiment, said one or more genes consists of genes encoding respectively 2, 3, 4, 5, 6, 7, 8, 9, 10, 11 or all of said protein kinases in said group.

In one embodiment, each said transcript level is at least 1.5-fold, 2-fold or 3-fold reduction from said threshold level.

In another embodiment, the method further comprises determining each said transcript level of said genes by a method comprising measuring the transcript level of each gene using one or more polynucleotide probes, each of said one or more polynucleotide probes comprising a nucleotide sequence complementary and hybridizable to a sequence in said gene. In one embodiment, said one or more polynucleotide probes are polynucleotide probes on a microarray.

In still another aspect, the invention provides a method for evaluating sensitivity of a cell to the growth inhibitory effect of an anti-cancer agent, said method comprising determining a level of abundance of a protein kinase selected from the group consisting of ATR, MAST2, MAP3K6, TBK1, ADRBK2, CDKL2, LATS2, STK32B, STK11, DDR1, PSKH2, and NEK8 in said cell, and determining whether said leve of abundance is below a predetermined threshold level, wherein said level of abundance of said protein kinase below said predetermined threshold level indicates that said cell is sensitive to the growth inhibitory effect of said anti-cancer agent.

In another embodiment, the invention provides a method for evaluating sensitivity of a cell to the growth inhibitory effect of an anti-cancer agent, said method comprising determining a level of activity of a protein kinase selected from the group consisting of ATR, MAST2, MAP3K6, TBK1, ADRBK2, CDKL2, LATS2, STK32B, STK11, DDR1, PSKH2, and NEK8, and determining whether said level of activity is below a predetermined threshold level, wherein said activity level below a predetermined threshold level indicates that said cell is sensitive to the growth inhibitory effect of said anti-cancer agent.

In one embodiment, said cell is an ex vivo cell. In another embodiment, said cell is an in vivo cell. In another embodiment, said cell is a human cell.

In one embodiment, said anti-cancer agent is selected from the group consisting of topoisomerase I inhibitor, topoisomerase II inhibitor, DNA binding agent, anti-metabolite, anti-mitotic agent, and ionizing radiation. In a specific embodiment, said anti-cancer agent is selected from the group consisting of camptothecin, cisplatin, gemcitabine, hydoxyurea, bleomycin, L-001000962-000Y, and 5-fluorouracil.

In still another aspect, the invention provides a method for enhancing sensitivity of a cell to an anti-cancer agent, comprising contacting said cell with an agent that reduces the expression of a gene encoding a protein kinase selected from the group consisting of ATR, MAST2, MAP3K6, TBK1, ADRBK2, CDKL2, LATS2, STK32B, STK11, DDR1, PSKH2, and NEK8 and/or the activity of said protein kinase, said agent being in an amount sufficient to enhance sensitivity of said cell to an anti-cancer agent.

In one embodiment, said agent comprises a substance selected from the group consisting of siRNA, antisense nucleic acid, ribozyme, and triple helix forming nucleic acid.

In another embodiment, said agent comprises a substance selected from the group consisting of antibody, peptide, and small molecule.

In one embodiment, said anti-cancer agent is selected from the group consisting of topoisomerase I inhibitor, topoisomerase II inhibitor, DNA binding agent, anti-metabolite, anti-mitotic agent, and ionizing radiation. In a specific embodiment, said anti-cancer agent is selected from the group consisting of camptothecin, cisplatin, gemcitabine, hydoxyurea, bleomycin, L-001000962-000Y, and 5-fluorouracil.

In still another aspect, the invention provides a method for regulating growth of a cell, comprising contacting said cell with i) a first agent that reduces the expression of a gene encoding a protein kinase selected from the group consisting of ATR, MAST2, MAP3K6, TBK1, ADRBK2, CDKL2, LATS2, STK32B, STK11, DDR1, PSKH2, and NEK8 and/or the activity of said protein kinase; and ii) an amount of an anti-cancer agent different from said first agent sufficient to regulate growth of said cell in the presence of said first agent. In one embodiment, said cell is an ex vivo cell. In another embodiment, said cell is an in vivo cell.

In one embodiment, said first agent comprises a substance selected from the group consisting of siRNA, antisense nucleic acid, ribozyme, and triple helix forming nucleic acid.

In another embodiment, said first agent comprises a substance selected from the group consisting of antibody, peptide, and small molecule.

In preferred embodiments, said first agent comprises an siRNA targeting said gene.

In one embodiment, said first agent comprises 2, 3, 4, 5, 6, or 10 different siRNAs targeting said gene. In one embodiment, the total siRNA concentration of said different siRNAs in said first agent is an optimal concentration for silencing said gene, wherein said optimal concentration is a concentration further increase of which does not increase the level of silencing substantially. The optimal concentration can be a concentration further increase of which does not increase the level of silencing by more than 20%, more than 10%, or more than 5%. In one embodiment, the concentration of each said different siRNA is about the same. In another embodiment, the respective concentrations of said different siRNAs are different from each other by less than 50%, less than 20%, or less than 10% of said total siRNA concentration of said different siRNAs. In still another embodiment, none of the siRNAs in said first agent has a concentration that is more than 80%, more than 50%, or more than 20% of said total siRNA concentration of said different siRNAs. In still another embodiment, at least one siRNA in said first agent has a concentration that is more than 20% or more than 50% of said total siRNA concentration of said different siRNAs. In still another embodiment, the number of different siRNAs and the concentration of each siRNA in said first agent is chosen such that said agent causes less than 10%, less than 1%, less than 0.1%, or less than 0.01% of silencing of any off-target genes.

In still another aspect, the invention provides a method of identifying a substance that is capable of enhancing sensitivity of a cell to the growth inhibitory effect of an anti-cancer agent, wherein said first agent is capable of reducing the expression of a gene encoding a protein kinase selected from the group consisting of ATR, MAST2, MAP3K6, TBK1, ADRBK2, CDKL2, LATS2, STK32B, STK11, DDR1, PSKH2, and NEK8 and/or the activity of said protein kinase, said method comprising comparing a growth inhibitory effect of an anti-cancer agent on cells expressing said gene in the presence of each of one or more candidate substances with the growth inhibitory effect of said anti-cancer agent on cells expressing said gene in the absence of said one or more candidate substances, wherein a greater growth inhibitory effect of said anti-cancer agent in the presence of said one or more candidate substances identifies said one or more candidate substances as capable of enhancing sensitivity of said cell to the growth inhibitory effect of said anti-cancer agent.

In a specific embodiment, the method comprises (a) contacting a first cell of said cell type expressing said first gene with said anti-cancer agent in the presence of said candidate substance and measuring a first growth inhibitory effect; (b) contacting a second cell of said cell type expressing said first gene with said anti-cancer agent under the same conditions as (a) except in the absence of said candidate substance and measuring a second growth inhibitory effect; and (c) comparing said first and second growth inhibitory effects measured in said step (a) and (b), wherein a greater first growth inhibitory effect than said second growth inhibitory effect identifies said candidate substance as capable of enhancing sensitivity of a cell to the growth inhibitory effect of said anti-cancer agent.

In one embodiment, said cell is an ex vivo cell. In another embodiment, said cell is an in vivo cell.

In one embodiment, said cell expresses an siRNA targeting a second target gene. In a specific embodiment, said second target gene is p53.

In one embodiment, said substance comprises a molecule that reduces expression of said gene.

In preferred embodiments, said substance comprises an siRNA targeting said gene. In one embodiment, said substance comprises 2, 3, 4, 5, 6, or 10 different siRNAs targeting said gene. In one embodiment, the total siRNA concentration of said different siRNAs in said substance is an optimal concentration for silencing said gene, wherein said optimal concentration is a concentration further increase of which does not increase the level of silencing substantially. The optimal concentration can be a concentration further increase of which does not increase the level of silencing by more than 20%, more than 10%, or more than 5%. In one embodiment, the concentration of each said different siRNA is about the same. In another embodiment, the respective concentrations of said different siRNAs are different from each other by less than 50%, less than 20%, or less than 10% of said total siRNA concentration of said different siRNAs. In still another embodiment, none of the siRNAs in said substance has a concentration that is more than 80%, more than 50%, or more. than 20% of said total siRNA concentration of said different siRNAs. In still another embodiment, at least one siRNA in said substance has a concentration that is more than 20% or more than 50% of said total siRNA concentration of said different siRNAs. In still another embodiment, the number of different siRNAs and the concentration of each siRNA in said substance is chosen such that said agent causes less than 10%, less than 1%, less than 0.1%, or less than 0.01% of silencing of any off-target genes.

In some embodiments, said anti-cancer agent is selected from the group consisting of a topoisomerase I inhibitor, a topoisomerase II inhibitor, a DNA binding agent, anti-metabolite, anti-mitotic agent, and ionizing radiation. In a specific embodiment, said anti-cancer agent is selected from the group consisting of camptothecin, cisplatin, gemcitabine, hydoxyurea, bleomycin, L-001000962-000Y, and 5-fluorouracil.

In still another aspect, the invention provides microarray for determining sensitivity of a cell to the growth inhibitory effect of an anti-cancer agent, said microarray comprising (i) one or more first polynucleotide probes attached to a substrate in a positionally addressable manner, wherein each of said first polynucleotide probes comprises a nucleotide sequence complementary and hybridizable to a sequence in a gene encoding a different protein kinase selected from the group consisting of ATR, MAST2, MAP3K6, TBK1, ADRBK2, CDKL2, LATS2, STK32B, STK11, DDR1, PSKH2, and NEK8; wherein said first polynucleotide probes are at least 50%, 75%, 80%, 85%, 90%, 95%, 97%, 98%, 99% or 100% of the total polynucleotide probes attached to said substrate in a positionally addressable manner; and (ii) optional control probes.

In still another aspect, the invention provides a kit comprising in separate containers a plurality of first polynucleotide probes, wherein each said first polynucleotide probe comprises a nucleotide sequence complementary and hybridizable to a sequence a gene encoding a protein kinase selected from the group consisting of ATR, MAST2, MAP3K6, TBK1, ADRBK2, CDKL2, LATS2, STK32B, STK11, DDR1, PSKH2, and NEK8; wherein said first polynucleotide probes are at least 50%, 75%, 80%, 85%, 90%, 95%, 97%, 98%, 99% or 100% of the total polynucleotide probes in said kit.

In still another aspect, the invention provides a kit comprising in one or separate containers (i) a protein kinase selected from the group consisting of ATR, MAST2, MAP3K6, TBK1, ADRBK2, CDKL2, LATS2, STK32B, STK11, DDR1, PSKH2, and NEK8, or a peptide fragment thereof; and (ii) said anti-cancer agent.

In still another aspect, the invention provides a kit comprising in one or separate containers (i) a first agent that reduces the expression of a gene encoding a protein kinase selected from the group consisting of ATR, MAST2, MAP3K6, TBK1, ADRBK2, CDKL2, LATS2, STK32B, STK11, DDR1, PSKH2, and NEK8, and/or the activity of said protein kinase; and (ii) a therapeutically effective amount of an anti-cancer agent different from said first agent. In one embodiment, said first agent comprises a substance selected from the group consisting of siRNA, antisense nucleic acid, ribozyme, and triple helix forming nucleic acid. In another embodiment, said first agent comprises an siRNA targeting said gene. In still another embodiment, said first agent comprises 2, 3, 4, 5, 6, or 10 different siRNAs targeting said gene. In one embodiment, the first agent and the anti-cancer agent are each purified.

In still another embodiment, said anti-cancer agent is selected from the group consisting of a topoisomerase I inhibitor, a topoisomerase II inhibitor, a DNA binding agent, anti-metabolite, anti-mitotic agent, and ionizing radiation. In a specific embodiment, said anti-cancer agent is selected from the group consisting of camptothecin, cisplatin, gemcitabine, hydoxyurea, bleomycin, L-001000962-000Y, and 5-fluorouracil.

In still another aspect, the invention provides a pharmaceutical composition, comprising (i) an agent that reduces the expression of a gene encoding a protein kinase selected from the group consisting of ATR, MAST2, MAP3K6, TBK1, ADRBK2, CDKL2, LATS2, STK32B, STK11, DDR1, PSKH2, and NEK8, and/or the activity of said protein kinase; and (ii) a pharmaceutically acceptable carrier. In one embodiment, said agent comprises a substance selected from the group consisting of siRNA, antisense nucleic acid, ribozyme, and triple helix forming nucleic acid. In another embodiment, said agent comprises an siRNA targeting said gene. In still another embodiment, said agent comprises 2, 3, 4, 5, 6, or 10 different siRNAs targeting said gene. In still another embodiment, said agent is purified.

### 4. DETAILED DESCRIPTION OF THE INVENTION

The invention provides methods and compositions for utilizing genes encoding certain protein kinases and/or their gene products, e.g., the encoded mRNA or kinases, in treating cancer in a patient. As used herein, a patient is an animal. The patient can be, but is not limited to, a human, or, in a veterinary context, a non-human animal such as a mammal, primate, ruminant, horse, swine or sheep, or a domestic companion animal such as a feline or canine. The protein kinases include ATR, MAST2, MAP3K6, TBK1, ADRBK2, CDKL2, LATS2, STK32B, STK11, DDR1, PSKH2, and NEK8. In the application, for simplicity reasons, these kinases are often referred to as PKs. A gene encoding such a kinase is also referred to as a “PK gene.” As used herein, the term “gene product” includes mRNA transcribed from the gene and protein encoded by the gene. The invention is based, at least in part, on the identification of the involvement of these kinases in the response of tumor cells to anti-cancer drugs using RNA interference (RNAi) screens. Tumor cells in which one or more of these genes were silenced by RNAi exhibited enhanced response to anti-cancer drugs. In this application, a cell type refers to a particular type of cell, e.g., a type of cell that can be distinguished from other types of cells by one or more phenotypic and/or genotypic characteristics. For example, a cell type can be but is not limited to a particular cell line, e.g., a tumor cell line, a particular tissue type, e.g., liver cell, erythrocyte, etc.

Thus, the invention provides methods and compositions for treating cancer by modulating, e.g., reducing, the expression and/or activity of the PK genes and/or their gene products, and/or by modulating interactions of the PK genes and/or their gene products with other proteins or molecules, e.g., substrates. The methods and compositions can be used for modulating, e.g., enhancing, the sensitivity of cells to the growth inhibitory effect of an anti-cancer agent, thus enhancing the growth inhibitory effect of the anti-cancer agent in a cell or organism. Thus, such methods and compositions can be used in conjunction with an anti-caner drug to enhance the effect of chemotherapy. In one embodiment, the expression of one or more of the PK genes is reduced to enhance the effects of anti-cancer agents. Such modulation can be achieved by, e.g., using nucleic acids, antisense nucleic acid, ribozyme, triple helix forming nucleic acid, and/or siRNAs that target the PK genes. In another embodiment, the activity of one or more PKs, e.g., the catalytic activities of the PKs and/or the interaction of the PKs with other intra- or extra-cellular molecules, are reduced to enhance the effects of anti-cancer agents. Such modulation can be achieved by, e.g., using antibodies, peptide molecules, and/or small organic or inorganic molecules that target a PK.

The invention also provides methods and compositions for diagnosing resistance or sensitivity of a cell to the growth inhibitory effect of anti-cancer agents based on the expression and/or activity level of one or more of the PK genes or the encoded PKs. The invention also provides methods and composition for assigning treatment regimen for a cancer patient based on one or more of the PK genes and/or gene products. The invention also provides methods and composition for monitoring treatment progress for a cancer patient based on the status of one or more of the PK genes or gene products.

The invention also provides methods and compositions for enhancing the sensitivity of cancer cells to the growth inhibitory effect of an anti-cancer drug by reducing the expression and/or activity of one or more of ATR, MAST2, MAP3K6, TBK1, ADRBK2, CDKL2, LATS2, STK32B, STK11, DDR1, PSKH2, and NEK8 genes, and/or the encoded kinases. The cell or cells can be ex vivo, e.g., in a cell culture, or in vivo. The anti-cancer drugs can be but are not limited to those described in Section 4.3.

The invention also provides methods and compositions for utilizing these genes and their products for screening for agents that modulate their expression and/or activity and/or modulating their interactions with other proteins or molecules. Such agents can be used to enhance the response of cells to anti-cancer agents. Thus, the invention provides methods and compositions for utilizing these genes and gene products for screening for agents that are useful in enhancing sensitivity of cells to the growth inhibitory effect of anti-cancer agents and/or in enhancing the growth inhibitory effect of anti-cancer agent in a cell or organism. The compositions of the invention include but not limited to nucleic acid, antisense nucleic acid, ribozyme, triple helix forming nucleic acid, siRNA, antibody, peptide or polypeptide molecules, and small organic or inorganic molecules.

The present invention also provides methods and compositions for identifying other extra- or intra-cellular molecules, e.g., genes and proteins, which interact with the PK genes and/or their gene products, and/or pathways in which the PKs are involved. The present invention also provides methods and compositions for modulating response of a cell to anti-cancer agents and/or tumorgenesis by modulating such cellular constituents and/or pathways. In specific embodiments of the invention, the present invention provides methods and compositions for modulating response of a cell to anti-cancer agents and/or tumorgenesis in a cell or organism by targeting one or more of the cellular constituents that interact with a PK gene and/or corresponding gene products. In one embodiment of the invention, response of a cell to anti-cancer agents and/or tumorgenesis is modulated, e.g., enhanced, by modulating the expression and/or activity of such cellular constituent.

### 4.1. Cancer Therapy by Kinase Genes and/or Their Products

The invention provides methods and compositions for utilizing the kinases listed in Table 1 in treating cancer. In particular, the invention provides methods and compositions for enhancing the sensitivity of cells to the growth inhibitory effect of anti-cancer drugs by reducing the expression and/or activity of such kinases and/or the genes encoding the kinases. The methods and composition can be used in conjunction with one or more anti-cancer drugs, e.g., the anti-cancer agents described in Section 4.3., infra, to treat cancers. The compositions (i.e., agents that reduce expression and/or activity of the kinase) of the invention are preferably purified.

TABLE 1
PKs of the invention

| | | NCBI REFSEQ | | |
|---|---|---|---|---|
| KINASE | ABREVIATION | ACCESSION NO. | GROUP | FAMILY |
| Ataxia-telangiectasia- | ATR | NM_001184 | Atypical | PIKK |
| and Rad3-related kinase | | | | |
| Microtubule associated | MAST2 | NM_015112 | AGC | MAST |
| serine/threonine kinase 2 | | | | |
| Mitogen-activated protein | MAP3K6 | NM_004672 | STE | STE11 |
| kinase kinase kinase 6 | | | | |
| TANK-binding kinase 1 | TBK1 | NM_013254 | Other | IKK |
| Adrenergic, beta, receptor | ADRBK2 | NM_005160 | AGC | GRK |
| kinase 2 | | | | |
| Cyclin-dependent kinase- | CDKL2 | NM_003948 | CMGC | CDKL |
| like 2 | | | | |
| Large tumor suppressor, | LATS2 | NM_014572 | AGC | NDR |
| homolog 2 | | | | |
| Serine/threonine kinase | STK32B | NM_018401 | AGC | YANK |
| 32B | | | | |
| Serine/threonine kinase | STK11 | NM_000455 | CAMK | CAMKL |
| 11 | | | | |
| Discoidin domain | DDR1 | NM_013994 | TK | DDR |
| receptor 1 kinase | | | | |
| Protein serine kinase H2 | PSKH2 | NM_033126 | CAMK | PSK |
| NIMA (never in mitosis | NEK8 | NM_178170 | Other | NEK |
| gene a)-related kinase 8 | | | | |

In one embodiment, the invention provides method and compositions for regulating growth of a cell, comprising contacting the cell with a first agent that reduces the expression of a gene encoding a protein kinase selected from the group consisting of ATR, MAST2, MAP3K6, TBK1, ADRBK2, CDKL2, LATS2, STK32B, STK11, DDR1, PSKH2, and NEK8 and/or the activity of said protein kinase; and an amount of an anti-cancer agent different from said first agent sufficient to regulate growth of the cell in the presence of said first agent. In one embodiment, the methods and compositions are used to regulate growth of cells ex vivo (e.g., in a cell culture). In another embodiment, the method and compositions are used to regulate growth of cells in vivo, e.g., by administering the first agent and the anti-cancer agent to a patient.

In one embodiment, the invention provides methods and compositions for enhancing the sensitivity of cancer cells to the growth inhibitory effect of an anti-cancer drug by at least 2 fold, 3 fold, 4 fold, 6 fold, 8 fold or 9 fold by reducing the expression and/or activity of one or more of ATR, MAST2, MAP3K6, TBK1, ADRBK2, CDKL2, LATS2, STK32B, STK11, DDR1, PSKH2, and NEK8 genes, and/or the encoded kinases. The cell or cells can be ex vivo, e.g., in a cell culture, or in vivo. The anti-cancer drugs can be but are not limited to those described in Section 4.3. In one embodiment, the expression level of one or more such genes in a cancer cell is reduced to enhance the sensitivity of the cell to the growth inhibitory effect of an anti-cancer drug. In another embodiment, the level of abundance of one or more such kinases in a cancer cell is reduced to enhance the sensitivity of the cell to the growth inhibitory effect of an anti-cancer drug. In still another embodiment, the activity of one or more such kinases in a cancer cell, e.g., substrate binding, ATP bindng, or the binding to a signal molecule, is reduced or inhibited to enhance the sensitivity of the cell to the growth inhibitory effect of an anti-cancer drug.

The invention provides methods and compositions for enhancing the sensitivity of cancer cells to the growth inhibitory effect of specific anti-cancer drugs. In one embodiment, the invention provides methods and compositions for enhancing the sensitivity of cells to the growth inhibitory effect of 5-fluorouracil by reducing the expression and/or activity of ADRBK2 and/or STK32B and/or the encoded kinases.

In still another embodiment, the invention provides methods and compositions for enhancing the sensitivity of cells to the growth inhibitory effect of cisplatin by reducing the expression and/or activity of one or more of the following kinase genes: ATR, MAST2, MAP3K6, TBK1, ADRBK2, CDKL2, LATS2, STK32B, STK11, DDR1, PSKH2, and NEK8, and/or the encoded kinases.

In still another embodiment, the invention provides methods and compositions for enhancing the sensitivity of cells to the growth inhibitory effect of bleomycin by reducing the expression and/or activity of one or more of the following kinase genes: ATR, MAST2, MAP3K6, and ADRBK2, and/or the encoded kinases.

In still another embodiment, the invention provides methods and compositions for enhancing the sensitivity of cells to the growth inhibitory effect of gemcitabine by reducing the expression and/or activity of CDKL2 and/or LATS2, and/or the encoded kinases.

In still another embodiment, the invention provides methods and compositions for enhancing the sensitivity of cells to the growth inhibitory effect of hydroxyurea by reducing the expression and/or activity of one or more of ATR, MAST2, and LATS2, and/or the encoded kinases.

In still another embodiment, the invention provides methods and compositions for enhancing the sensitivity of cells to the growth inhibitory effect of a KSP inhibitor (1S)-1-{[(2S)-4-(2,5-difluorophenyl)-2-phenyl-2,5-dihydro-1H-pyrrol-1-yl]carbonyl}-2-methylpropylamine (“L-001000962-000Y” or “KSPi”) (see, PCT application publication WO 03/105,855, published on Dec. 24, 2003, which is incorporated herein by reference in its entirety) by reducing the expression and/or activity of one of more of CDKL2, LATS2, and DDR1, and/or the encoded kinases.

In still another embodiment, the invention provides methods and compositions for enhancing the sensitivity of cells to the growth inhibitory effect of camptothecin by reducing the expression and/or activity of DDR1, and/or the encoded kinases.

In other embodiments, the invention provides methods and compositions for treating cancer in a patient by reducing the expression and/or activity of one or more of ATR, MAST2, MAP3K6, TBK1, ADRBK2, CDKL2, LATS2, STK32B, STK11, DDR1, PSKH2, and NEK8 genes, and/or the encoded kinases in combination with administration of an anti-cancer agent.

In a specific embodiment, the invention provides a method for treating cancer in a patient by administering to a patient (i) an agent that reduces the expression and/or activity of ADRBK2 and/or STK32B and/or the encoded kinases, and (ii) a therapeutically effective amount of 5-fluorouracil.

In another specific embodiment, the invention provides a method for treating cancer in a patient by administering to a patient (i) an agent that reduces the expression and/or activity of one or more of ATR, MAST2, MAP3K6, TBK1, ADRBK2, CDKL2, LATS2, STK32B, STK11, DDR1, PSKH2, and NEK8, and/or the encoded kinases, and (ii) a therapeutically effective amount of cisplatin.

In still another specific embodiment, the invention provides a method for treating cancer in a patient by administering to a patient (i) an agent that reduces the expression and/or activity of one or more of ATR, MAST2, MAP3K6, and ADRBK2, and/or the encoded kinases, and (ii) a therapeutically effective amount of bleomycin.

In still another specific embodiment, the invention provides a method for treating cancer in a patient by administering to a patient (i) an agent that reduces the expression and/or activity of CDKL2 and/or LATS2, and/or the encoded kinases, and (ii) a therapeutically effective amount of gemcitabine.

In still another specific embodiment, the invention provides a method for treating cancer in a patient by administering to a patient (i) an agent that reduces the expression and/or activity of one or more of ATR, MAST2, and LATS2, and/or the encoded kinases, and (ii) a therapeutically effective amount of hydroxyurea.

In still another specific embodiment, the invention provides a method for treating cancer in a patient by administering to a patient (i) an agent that reduces the expression and/or activity of one or more of CDKL2, LATS2, and DDR1, and/or the encoded kinases, and (ii) a therapeutically effective amount of L-001000962-000Y.

In still another specific embodiment, the invention provides a method for treating cancer in a patient by administering to a patient (i) an agent that reduces the expression and/or activity of DDR1, and/or the encoded kinases, and (ii) a therapeutically effective amount of camptothecin.

In another specific embodiment, the invention provides a method for treating cancer in a patient by administering to a patient (i) an agent that reduces the expression and/or activity of AIR and/or the encoded kinase, and (ii) a therapeutically effective amount of one or more of cisplatin, bleomycin, and hydroxyurea.

In still another specific embodiment, the invention provides a method for treating cancer in a patient by administering to a patient (i) an agent that reduces the expression and/or activity of MAST2 and/or the encoded kinase, and (ii) a therapeutically effective amount of one or more of cisplatin, bleomycin, and hydroxyurea.

In another specific embodiment, the invention provides a method for treating cancer in a patient by administering to a patient (i) an agent that reduces the expression and/or activity of MAP3K6 and/or the encoded kinase, and (ii) a therapeutically effective amount of cisplatin and/or bleomycin.

In another specific embodiment, the invention provides a method for treating cancer in a patient by administering to a patient (i) an agent that reduces the expression and/or activity of ADRBK2 and/or the encoded kinase, and (ii) a therapeutically effective amount of one or more of cisplatin, bleomycin, and 5-fluorouracil.

In another specific embodiment, the invention provides a method for treating cancer in a patient by administering to a patient (i) an agent that reduces the expression and/or activity of CDKL2 and/or the encoded kinase, and (ii) a therapeutically effective amount of one or more of cisplatin, gemcitabine, and L-001000962-000Y.

In another specific embodiment, the invention provides a method for treating cancer in a patient by administering to a patient (i) an agent that reduces the expression and/or activity of LATS2 and/or the encoded kinase, and (ii) a therapeutically effective amount of one or more of cisplatin, hydroxyurea, gemcitabine, and L-001000962-000Y.

In another specific embodiment, the invention provides a method for treating cancer in a patient in a patient by administering to a patient (i) an agent that reduces the expression and/or activity of STK32B and/or the encoded kinase, and (ii) a therapeutically effective amount of cisplatin and/or 5-fluorouracil.

In another specific embodiment, the invention provides a method for treating cancer in a patient by administering to a patient (i) an agent that reduces the expression and/or activity of DDR1 and/or the encoded kinase, and (ii) a therapeutically effective amount of one or more of cisplatin, camptothecin, and L-001000962-000Y.

A variety of therapeutic approaches may be used in accordance with the invention to reduce expression of the PK gene of the invention in vivo. For example, siRNA molecules may be engineered and used to silence a PK gene of the invention in vivo. Antisense DNA molecules may also be engineered and used to block translation of a PK mRNA in vivo. Alternatively, ribozyme molecules may be designed to cleave and destroy the PK mRNAs in vivo. In another alternative, oligonucleotides designed to hybridize to the 5′ region of the PK gene of the invention (including the region upstream of the coding sequence) and form triple helix structures may be used to block or reduce transcription of the PK gene of the invention.

In a preferred embodiment, RNAi is used to knock down the expression of a PK gene of the invention, i.e., a gene encoding one of the following protein kinases ATR, MAST2, MAP3K6, TBK1, ADRBK2, CDKL2, LATS2, STK32B, STK11, DDR1, PSKH2, and NEK8. In one embodiment, double-stranded RNA molecules of 21-23 nucleotides which hybridize to a homologous region of mRNAs transcribed from the PK gene are used to degrade the mRNAs, thereby “silencing” the expression of the PK gene. Preferably, the dsRNAs have a hybridizing region, e.g., a 19-nucleotide double-stranded region, which is complementary to a sequence of the coding sequence of the PK gene. Any siRNA or a pool (mixture) of siRNAs that targets an appropriate coding sequence of a PK gene and exhibits a sufficient level of silencing can be used in the invention. As exemplary embodiments, 21-nucleotide double-stranded siRNAs targeting the coding regions of a PK gene are designed according to selection rules known in the art (see, e.g., Elbashir et al., 2002, Methods 26:199-213; International Application Publication No. WO 2005/042708, published May 12, 2005, each of which is incorporated herein by reference in its entirety). In a preferred embodiment, the siRNA or siRNAs specifically inhibit the translation or transcription of a PK gene without substantially affecting the translation or transcription of genes encoding other protein kinases in the same kinase family. In a specific embodiment, siRNAs targeting LATS2 specifically inhibit the translation or transcription of LATS2 without substantially affecting the translation or transcription of the gene encoding LATS1. In specific embodiments, siRNAs listed in Table 2 are used to silence the respective PK genes.

RNAi can be carried out using any standard method for introducing nucleic acids into cells. In one embodiment, gene silencing is induced by presenting the cell with one or more siRNAs targeting the PK gene (see, e.g., Elbashir et al., 2001, Nature 411, 494-498; Elbashir et al., 2001, Genes Dev. 15, 188-200, all of which are incorporated by reference herein in their entirety). The siRNAs can be chemically synthesized, or derived from cleavage of double-stranded RNA by recombinant Dicer. Another method to introduce a double stranded DNA (dsRNA) for silencing of the PK gene of the invention is shRNA, for short hairpin RNA (see, e.g., Paddison et al., 2002, Genes Dev. 16, 948-958; Brummelkamp et al., 2002, Science 296, 550-553; Sui, G. et al. 2002, Proc. Natl. Acad. Sci. USA 99, 5515-5520, all of which are incorporated by reference herein in their entirety). In this method, a siRNA targeting a PK gene of the invention is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid- or virus-based shRNAs can be expressed stably in cells, allowing long-term gene silencing in cells both in vitro and in vivo (see, McCaffrey et al. 2002, Nature 418, 38-39; Xia et al., 2002, Nat. Biotech. 20, 1006-1010; Lewis et al., 2002, Nat. Genetics 32, 107-108; Rubinson et al., 2003, Nat. Genetics 33, 401-406; Tiscornia et al., 2003, Proc. Natl. Acad. Sci. USA 100, 1844-1848, all of which are incorporated by reference herein in their entirety). Such plasmid- or virus-based shRNAs can be delivered using a gene therapy approach. SiRNAs targeting the PK gene of the invention can also be delivered to an organ or tissue in a mammal, such a human, in vivo (see, e.g., Song et al. 2003, Nat. Medicine 9, 347-351; Sorensen et al., 2003, J. Mol. Biol. 327, 761-766; Lewis et al., 2002, Nat. Genetics 32, 107-108, all of which are incorporated by reference herein in their entirety). In this method, a solution of siRNA is injected intravenously into the mammal. The siRNA can then reach an organ or tissue of interest and effectively reduce the expression of the target gene in the organ or tissue of the mammal.

In preferred embodiments, an siRNA pool containing at least k (k=2, 3, 4, 5, 6 or 10) different siRNAs targeting the same PK gene at different sequence regions is used to silence the gene. In a preferred embodiment, the total siRNA concentration of the pool is about the same as the concentration of a single siRNA when used individually. As used herein, the word “about” with reference to concentration means within 20%. Preferably, the total concentration of the pool of siRNAs is an optimal concentration for silencing the intended target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the composition of the pool, including the number of different siRNAs in the pool and the concentration of each different siRNA, is chosen such that the pool of siRNAs causes less than 30%, 20%, 10% or 5%, 1%, 0.1% or 0.01% of silencing of any off-target genes (e.g., as determined by standard nucleic acid assay, e.g., PCR). In another preferred embodiment, the concentration of each different siRNA in the pool of different siRNAs is about the same. In still another preferred embodiment, the respective concentrations of different siRNAs in the pool are different from each other by less than 5%, 10%, 20% or 50% of the concentration of any one siRNA or said total siRNA concentration of said different siRNAs. In still another preferred embodiment, at least one siRNA in the pool of different siRNAs constitutes more than 90%, 80%, 70%, 50%, or 20% of the total siRNA concentration in the pool. In still another preferred embodiment, none of the siRNAs in the pool of different siRNAs constitutes more than 90%, 80%, 70%, 50%, or 20% of the total siRNA concentration in the pool. In other embodiments, each siRNA in the pool has a concentration that is lower than the optimal concentration when used individually. In a preferred embodiment, each different siRNA in the pool has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the pool has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene. In specific embodiments, a pool containing the 3 different siRNAs listed in Table 2, infra, is used for targeting a corresponding PK gene. More detailed descriptions of techniques for carrying out RNAi are also presented in Section 4.5.

In other embodiments, antisense nucleic acid, ribozyme, and triple helix forming nucleic acid nucleotides are designed to inhibit the translation or transcription of a PK with minimal effects on the expression of other genes that may share one or more sequence motif with the PK gene. To accomplish this, the oligonucleotides used should be designed on the basis of relevant sequences unique to a PK gene. In one embodiment, the oligonucleotide used specifically inhibits the translation or transcription of a PK without substantially affecting the translation or transcription of other protein kinases in the same kinase family.

For example, and not by way of limitation, the oligonucleotides should not fall within those regions where the nucleotide sequence of a PK gene is most homologous to that of other genes. In the case of antisense molecules, it is preferred that the sequence be at least 18 nucleotides in length in order to achieve sufficiently strong annealing to the target mRNA sequence to prevent translation of the sequence. See, Izant et al., 1984, Cell, 36:1007-1015; Rosenberg et al., 1985, Nature, 313:703-706.

Ribozymes are RNA molecules which possess highly specific endoribonuclease activity Hammerhead ribozymes comprise a hybridizing region which is complementary in nucleotide sequence to at least part of the target RNA, and a catalytic region which is adapted to cleave the target RNA. The hybridizing region contains nine (9) or more nucleotides.

Therefore, the hammerhead ribozymes useful for targeting a PK of the invention have a hybridizing region which is complementary to the sequences listed above and is at least nine nucleotides in length. The construction and production of such ribozymes is well known in the art and is described more fully in Haseloff et al., 1988, Nature, 334:585-591.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been et al., 1986, Cell, 47:207-216). The Cech endoribonucleases have an eight base pair active site which hybridizes to a target RNA sequence whereafter cleavage of the target RNA takes place.

In the case of oligonucleotides that hybridize to and form triple helix structures at the 5′ terminus of a PK gene of the invention and can be used to block transcription, it is preferred that they be complementary to those sequences in the 5′ terminus of a PK gene which are not present in other related genes. It is also preferred that the sequences not include those regions of the PK promoter which are even slightly homologous to that of other related genes.

The foregoing compounds can be administered by a variety of methods which are known in the art including, but not limited to the use of liposomes as a delivery vehicle. Naked DNA or RNA molecules may also be used where they are in a form which is resistant to degradation such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to poly-lysine or transferrin. Nucleic acid may also be transported into cells by any of the various viral carriers, including but not limited to, retrovirus, vaccinia, AAV, and adenovirus.

Alternatively, a recombinant nucleic acid molecule which encodes, or is, such antisense, ribozyme, a molecule that can form a triple helix, or PK molecule can be constructed. This nucleic acid molecule may be either RNA or DNA. If the nucleic acid encodes an RNA, it is preferred that the sequence be operatively attached to a regulatory element so that sufficient copies of the desired RNA product are produced. The regulatory element may permit either constitutive or regulated transcription of the sequence. In vivo, that is, within the cells or cells of an organism, a transfer vector such as a bacterial plasmid or viral RNA or DNA, encoding one or more of the RNAs, may be transfected into cells e.g. (Llewellyn et al., 1987, J. Mol. Biol., 195:115-123; Hanahan et al. 1983, J. Mol. Biol., 166:557-580). Once inside the cell, the transfer vector may replicate, and be transcribed by cellular polymerases to produce the RNA or it may be integrated into the genome of the host cell. Alternatively, a transfer vector containing sequences encoding one or more of the RNAs may be transfected into cells or introduced into cells by way of micromanipulation techniques such as microinjection, such that the transfer vector or a part thereof becomes integrated into the genome of the host cell.

The activity of a PK of the invention can be modulated by modulating the interaction of the PK with its binding partners, including but not limited to its substrates, ATP, and signaling molecules. In one embodiment, agents, e.g., antibodies, peptides, aptamers, small organic or inorganic molecules, can be used to inhibit binding of a PK to its binding partner in a cell such that sensitivity of the cell to an anti-cancer agent is enhanced. In another embodiment, agents, e.g., antibodies, aptamers, small organic or inorganic molecules, can be used to inhibit the activity of a protein in a pathway regulated by a PK of the invention in a cell such that sensitivity of the cell to an anti-cancer agent is enhanced.

In other embodiments, the invention provides small molecule inhibitors of the PKs as anti-proliferating agents. A small molecule inhibitor is a low molecular weight inhibitor of phosphorylation by a PK. As used herein, a small molecule refers to an organic or inorganic molecule having a molecular weight is under 1000 Daltons, preferably in the range between 300 to 700 Daltons, which is not a nucleic acid molecule or a peptide molecule. The small molecule can be naturally occurring, e.g., extracted from plant or microorganisms, or non-naturally occurring, e.g., generated de novo by synthesis. A small molecule that is an inhibitor can be used to block PK-dependent cell proliferation and/or sensitize the tumor cells to the effects of other anti-cancer drug, e.g., anti-cancer agents. In one embodiment, the inhibitors are substrate mimics. In a preferred embodiment, the inhibitor of the PKs of the invention is an ATP mimic. In one embodiment, such an ATP mimic possesses at least two aromatic rings. In a preferred embodiment, the ATP mimic comprises a moiety that forms extensive contacts with residues lining the ATP binding cleft of the target PK and/or peptide segments just outside the cleft, thereby selectively blocking the ATP binding site of the target PK. Minor structural differences from ATP can be introduced into the ATP mimic based on the peptide segments just outside the cleft. Such differences can lead to specific hydrogen bonding and hydrophobic interactions with the peptide segments just outside the cleft.

In still other embodiments, antibodies that specifically bind the PKs of the invention are used as anti-proliferating agents. In a preferred embodiment, the invention provides antibodies specifically bind the extracellular domain of a receptor tyrosine kinase of the invention. Antibodies that specifically bind a target can be obtained using standard method known in the art, e.g., a method described in Section 4.8.

In one embodiment, an antibody-drug conjugate comprising an antibody that specifically binds a PK and an anti-cancer drug molecule is used as an anti-proliferating agent. The efficacy of the antibodies that target specific molecules expressed by tumor cells can be increased by attaching toxins to them. Existing immunotoxins based on bacterial toxins like pseudomonas exotoxin, plant exotoxin like ricin or radio-nucleotides can be used.

The toxins are chemically conjugated to a specific ligand such as the variable domain of the heavy or light chain of the monoclonal antibody. Normal cells lacking the cancer specific antigens are not targeted by the antibody.

In other embodiments, a peptide or peptidomimetic that interferes with the interaction of a PK with its interaction partner is used. A peptide preferably has a size of at least 5, 10, 15, 20 or 30 amino acids. Such a peptide or peptidomimetic can be designed by a person skilled in the art based on the sequence and structure of a PK. In one embodiment, a peptide or peptidomimetic that interferes with substrate binding of a PK is used. In another embodiment, a peptide or peptidomimetic that interferes with the binding of a signal molecule to a PK is used. In a preferred embodiment, the invention provides a peptide that binds the C-terminal region of a PK. In another embodiment, the invention provides a peptide that binds the N-terminal region of the PK. In some embodiments of the invention, a PK fragment or polypeptide of at least 5, 10, 20, 50, 100 amino acids in length is used. In a specific embodiment, a peptide or peptidomimetic that interferes with the interaction between STK6 (Aurora Kinase A) and LATS2 is used. In another specific embodiment, a peptide or peptidomimetic that inhibits tyrosine kinase dimerization by competing with target proteins is to used. The peptide can be prepared by standard method known in the art.

In another embodiment, a dominant negative mutant of a PK is used to reduce activity of a PK. Such a dominant negative mutant can be designed by a person skilled in the art based on the sequence and structure of a PK. In one embodiment, a dominant negative mutant that interferes with substrate binding of a PK is used. In another embodiment, a dominant negative mutant that interferes with the binding of a signal molecule to a PK is used. In a preferred embodiment, the invention provides a dominant negative mutant that comprises the C-terminal region of a PK. In another embodiment, the invention provides a dominant negative mutant that comprises the N-terminal region of the PK.

In one embodiment, gene therapy can be used for delivering any of the above described nucleic acid and protein/peptide therapeutics into tumor cells. Exemplary methods for carrying out gene therapy are described below. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, New York; and Kriegler, 1990, Gene Transfer and Expression, A Laboratory Manual, Stockton Press, New York.

In a preferred embodiment, the therapeutic comprises a nucleic acid that is part of an expression vector that expresses a the therapeutic nucleic acid or peptide/polypeptide in a suitable host. In particular, such a nucleic acid has a promoter operably linked to the coding region, said promoter being inducible or constitutive, and, optionally, tissue-specific. In another particular embodiment, a nucleic acid molecule is used in which the coding sequences and any other desired sequences are flanked by regions that promote homologous recombination at a desired site in the genome, thus providing for intrachromosomal expression of the PK nucleic acid (see e.g., Koller and Smithies, 1989, Proc. Natl. Acad. Sci. U.S.A. 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432) (which can be used to target cell types specifically expressing the receptors), etc. In another embodiment, a nucleic acid-ligand complex can be formed in which the ligand comprises a fusogcnic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. U.S.A. 86:8932-8935; Zijlstra et al., 1989, Nature 342:435-438).

In a specific embodiment, a viral vector that contains the PK nucleic acid is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The PK nucleic acid to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, to 1993, Curr. Opin. Genet. and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells.

Kozarsky and Wilson (1993, Current Opinion in Genetics and Development 3:499-503) present a review of adenovirus-based gene therapy. Bout et al. (1994, Human Gene Therapy 5:3-10) demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234.

Adeno-associated virus (AAV) has also been proposed for use in gene therapy (Walsh et al., 1993, Proc. Soc. Exp. Biol. Med. 204:289-300).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled person in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as

T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a nucleic acid is introduced into the cells such that it is expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention. Such stem cells include but are not limited to hematopoietic stem cells (HSC), stem cells of epithelial tissues such as the skin and the lining of the gut, embryonic heart muscle cells, liver stem cells (PCT Publication WO 94/08598), and neural stem cells (Stemple and Anderson, 1992, Cell 71:973-985).

Epithelial stem cells (ESCs) or keratinocytes can be obtained from tissues such as the skin and the lining of the gut by known procedures (Rheinwald, 1980, Meth. Cell Bio. 21 A:229). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Rheinwald, 1980, Meth. Cell Bio. 21A:229; Pittelkow and Scott, 1986, Mayo Clinic Proc. 61:771). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique which provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see e.g., Kodo et al., 1984, J. Clin. Invest. 73:1377-1384). The HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. U.S.A. 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The methods and/or compositions described above for modulating the expression and/or activity of a PK gene or PK may be used to treat a patient having a cancer in conjunction with an anti-cancer agent. Such therapies may be used to treat cancers, including but not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

In preferred embodiments, the methods and/or compositions (i.e., the agents that reduce the expression or activity of a protein kinase) of the invention are used in conjunction with an anti-cancer agent for treatment of a patient having a cancer which exhibits resistance to the anti-cancer agent mediated by a PK. In such embodiments, the expression and/or activity of the PK are reduced to confer to cancer cells sensitivity to the anti-cancer agent, thereby conferring or enhancing the efficacy of an anti-cancer therapy using the anti-cancer agent.

In a combination therapy, one or more compositions of the present invention can be administered before, at the same time as, or after the administration of an anti-cancer agent . In one embodiment, the compositions of the invention are administered before the administration of an anti-cancer agent (i.e., the agent that reduces expression or activity of a PK is for sequential or concurrent use with one or more anti-cancer agents). In one embodiment, the composition of the invention and an anti-cancer agent are administered in a sequence and within a time interval such that the composition of the invention and an anti-cancer agent can act together to provide an increased benefit than if they were administered alone. In another embodiment, the composition of the invention and an anti-cancer agent are administered sufficiently close in time so as to provide the desired therapeutic outcome. The time intervals between the administration of the compositions of the invention and an anti-cancer agent can be determined by routine experiments that are familiar to one skilled person in the art. In one embodiment, an anti-cancer agent is given to the patient after the PK level reaches a desirable threshold. The level of PK of the invention can be determined by using any techniques known in the art such as those described in Section 4.2., infra.

The composition of the invention and an anti-cancer agent can be administered simultaneously or separately, in any appropriate form and by any suitable route. In one embodiment, the composition of the invention and the anti-cancer agent are administered by different routes of administration. In an alternate embodiment, each is administered by the same route of administration. The composition of the invention and the anti-cancer agent can be administered at the same or different sites, e.g. arm and leg.

In various embodiments, such as those described above, the composition of the invention and an anti-cancer agent are administered less than 1 hour apart, at about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart, or no more than 1 week or 2 weeks or 1 month or 3 months apart. As used herein, the word about means within 10%. In other embodiments, the composition of the invention and an anti-cancer agent are administered 2 to 4 days apart, 4 to 6 days apart, 1 week apart, 1 to 2 weeks apart, 2 to 4 weeks apart, one month apart, 1 to 2 months apart, or 2 or more months apart. In preferred embodiments, the composition of the invention and an anti-cancer agent are administered in a time frame where both are still active. One skilled in the art would be able to determine such a time frame by determining the half life of each administered component. In separate or in the foregoing embodiments, the composition of the invention and an anti-cancer agent are administered less than 2 weeks, one month, six months, 1 year or 5 years apart.

In another embodiment, the compositions of the invention are administered at the same time or at the same patient visit, as the anti-cancer agent.

In still another embodiment, one or more of the compositions of the invention are administered both before and after the administration of an anti-cancer agent. Such administration can be beneficial especially when the anti-cancer agent has a longer half life than that of the one or more of the compositions of the invention used in the treatment.

In one embodiment, the anti-cancer agent is administered daily and the composition of the invention is administered once a week for the first 4 weeks, and then once every other week thereafter. In one embodiment, the anti-cancer agent is administered daily and the composition of the invention is administered once a week for the first 8 weeks, and then once every other week thereafter.

In certain embodiments, the composition of the invention and the anti-cancer agent are cyclically administered to a subject. Cycling therapy involves the administration of the composition of the invention for a period of time, followed by the administration of an anti-cancer agent for a period of time and repeating this sequential administration. Cycling therapy can reduce the development of resistance to one or more of the therapies, avoid or reduce the side effects of one of the therapies, and/or improve the efficacy of the treatment. In such embodiments, the invention contemplates the alternating administration of the composition of the invention followed by the administration of an anti-cancer agent 4 to 6 days later, preferable 2 to 4 days, later, more preferably 1 to 2 days later, wherein such a cycle may be repeated as many times as desired.

In certain embodiments, the composition of the invention and an anti-cancer agent are alternately administered in a cycle of less than 3 weeks, once every two weeks, once every 10 days or once every week. In a specific embodiment of the invention, one cycle can comprise the administration of an anti-cancer agent by infusion over 90 minutes every cycle, 1 hour every cycle, or 45 minutes every cycle. Each cycle can comprise at least 1 week of rest, at least 2 weeks of rest, at least 3 weeks of rest. In an embodiment, the number of cycles administered is from 1 to 12 cycles, more typically from 2 to 10 cycles, and more typically from 2 to 8 cycles.

It will be apparent to one skilled person in the art that any combination of different timing of the administration of the compositions of the invention and an anti-cancer agent can be used. For example, when the anti-cancer agent has a longer half life than that of the composition of the invention, it is preferable to administer the compositions of the invention before and after the administration of the anti-cancer agent.

The frequency or intervals of administration of the compositions of the invention depends on the desired PK level, which can be determined by any of the techniques known in the art, e.g., those techniques described infra. The administration frequency of the compositions of the invention can be increased or decreased when the PK level changes either higher or lower from the desired level.

The effects or benefits of administration of the compositions of the invention alone or to in conjunction with an anti-cancer agent can be evaluated by any methods known in the art, e.g., by methods that are based on measuring the survival rate, side effects, dosage requirement of the anti-cancer agent, or any combinations thereof If the administration of the compositions of the invention achieves any one or more benefits in a patient, such as increasing the survival rate, decreasing side effects, lowing the dosage requirement for the anti-cancer agent, the compositions of the invention are said to have augmented a chemotherapy, and the method is said to have efficacy.

### 4.2. Diagnostics

The invention also provides methods and compositions for evaluating the sensitivity of a cancer to the growth inhibitory effect of an anti-cancer drug based on the expression or activity level of one or more of ATR, MAST2, MAP3K6, TBK1, ADRBK2, CDKL2, LATS2, STK32B, STK11, DDR1, PSKH2, and NEK8 genes, and/or the encoded kinases. Such information can be used to determine a patient's responsiveness to treatment by the anti-cancer drug. For example, cancer patients who have a defective regulation of a PK gene, and therefore have a predisposition to resistance or sensitivity to the growth inhibitory effect of an anti-cancer agent, can be identified. Such information can also be used to determine whether a treatment regimen should include modulating one or more of these kinases. Thus, the invention provides methods and composition for assigning a treatment regimen for a cancer patient. The invention also provides methods and composition for monitoring treatment progress for a cancer patient based on the level of expression and/or activity of one or more of the PKs.

A variety of methods can be employed for the diagnostic and prognostic evaluation of patients for their sensitivity to the growth inhibitory effect of an anti-cancer agent utilizing the PKs of the invention. In one embodiment, the sensitivity of a patient to an anti-cancer drug is evaluated based on a profile comprising measurements of the expression level of one or more of ATR, MAST2, MAP3K6, TBK1, ADRBK2, CDKL2, LATS2, STK32B, STK11, DDR1, PSKH2, and NEK8 genes, and/or abundance or activity of the encoded kinases. One or more of these kinases having a level of expression or activity below a respective predetermined threshold indicate sensitivity to the anti-cancer drug.

In one embodiment, the method comprises determining the expression level of a PK gene in a cell, e.g., level of the mRNA encoded by the PK gene, and determining whether the expression level is below a predetermined threshold, where an expression level below the predetermined threshold level indicates that the cell is sensitive to one or more anti-cancer agents. The cell can be ex vivo, e.g., in a cell culture, or in vivo. Preferably, the predetermined threshold level is at least 2-fold, 4-fold, 8-fold, or 10-fold of the normal expression level of the PK gene. In another embodiment, the invention provides a method for evaluating sensitivity to one or more anti-cancer agents in a cell comprising determining a level of abundance of a protein encoded by a PK gene in the cell, and determining whether the level of abundance is below a predetermined threshold, where a level of abundance of the protein below the predetermined threshold level indicates that the cell is sensitive to the anti-cancer agents. In still another embodiment, the invention provides a method for evaluating sensitivity to one or more anti-cancer agents in a cell comprising determining a level of activity of a protein encoded by the PK gene in the cell and determining whether said level of activity is below a predetermined threshold, where an activity level below the predetermined threshold level indicates that the cell is sensitive to the anti-cancer agents. Such reduced activity may be a result of a mutation in the PK gene. Thus, the invention also provides a method for evaluating sensitivity of a cell to one or more anti-cancer agents comprising determining whether a mutation is present in a PK gene or a protein encoded by the PK gene in the cell, where the detection of a mutation that causes a reduction of the activity of the PK below a predetermined threshold level indicates that the cell is sensitive to the anti-cancer agents. Preferably, the predetermined threshold level of abundance or activity is at least 2-fold, 4-fold, 8-fold, or 10-fold below the normal level of abundance or activity of the PK. In all of the foregoing embodiments, and the embodiments described below, the cell can be ex vivo, e.g., in a cell culture, or in vivo.

The invention provides methods and compositions for evaluating sensitivity of cancer cells to the growth inhibitory effect of specific anti-cancer drugs. In one embodiment, the invention provides methods and compositions for evaluating the sensitivity of cancer cells to the growth inhibitory effect of 5-fluorouracil by detecting the levels of expression and/or activity of any one or more of ADRBK2, STK32B and their encoded kinases.

In still another embodiment, the invention provides methods and compositions for evaluating the sensitivity of cancer cells to the growth inhibitory effect of cisplatin by detecting the levels of expression and/or activity of any one or more of ATR, MAST2, MAP3K6, TBK1, ADRBK2, CDKL2, LATS2, STK32B, STK11, DDR1, PSKH2, and NEK8, and their encoded kinases.

In still another embodiment, the invention provides methods and compositions for evaluating the sensitivity of cancer cells to the growth inhibitory effect of bleomycin by detecting the levels of expression and/or activity of any one or more of the kinase genes ATR, MAST2, MAP3K6, and ADRBK2, and their encoded kinases.

In still another embodiment, the invention provides methods and compositions for evaluating the sensitivity of cancer cells to the growth inhibitory effect of gemcitabine by detecting the levels of expression and/or activity of any one or more of CDKL2, LATS2, and their encoded kinases.

In still another embodiment, the invention provides methods and compositions for evaluating the sensitivity of cancer cells to the growth inhibitory effect of hydroxyurea by detecting the levels of expression and/or activity of any one or more of ATR, MAST2, and LATS2, and their encoded kinases.

In still another embodiment, the invention provides methods and compositions for evaluating the sensitivity of cancer cells to the growth inhibitory effect of L-001000962-000Y by detecting the levels of expression and/or activity of any one or more of CDKL2, LATS2, and DDR1, and their encoded kinases.

In still another embodiment, the invention provides methods and compositions for evaluating the sensitivity of cancer cells to the growth inhibitory effect of camptothecin by detecting the levels of expression and/or activity of DDR1, and/or its encoded kinase.

Such methods may, for example, utilize reagents such as the PK nucleotide sequences and antibodies directed against PKs, including peptide fragments thereof. Specifically, such reagents may be used, for example, for: (1) the detection of the presence of a mutation in a PK gene of the invention, or the detection of either over- or under-expression of the PK gene relative to the normal expression level; and (2) the detection of either an over- or an under-abundance of a PK relative to the normal PK level.

The methods described herein may be performed, for example, by utilizing pre-packaged diagnostic kits comprising nucleic acid or antibody to at least one specific PK gene of the invention.

Nucleic acid-based detection techniques are described, below, in Section 4.2.1. Peptide detection techniques are described, below, in Section 4.2.2.

### 4.2.1. Detection of Expression of a PK Gene

The expression of a PK gene in cells or tissues, e.g., the cellular level of transcripts of a PK of the invention and/or the presence or absence of mutations, can be detected utilizing a number of techniques. Nucleic acid from any nucleated cell can be used as the starting point for such assay techniques, and may be isolated according to standard nucleic acid preparation procedures which are well known to those of skill in the art. For example, the expression level of the PK gene of the invention can be determined by measuring the expression level of the PK gene of the invention using one or more polynucleotide probes, each of which comprises a nucleotide sequence complementary and hybridizable to a sequence in the PK gene of the invention. In particularly preferred embodiments of the invention, the method is used to diagnose sensitivity of a cancer to a treatment using an anti-cancer agent in a human by assaying the expression/activity of one or more of PK genes and/or their encoded PKs in a sample from the human.

DNA may be used in hybridization or amplification assays of biological samples to detect abnormalities involving structure, including point mutations, insertions, deletions and chromosomal rearrangements in a PK gene. Such assays may include, but are not limited to, Southern analyses, single stranded conformational polymorphism analyses (SSCP), DNA microarray analyses, and PCR analyses.

Such diagnostic methods for the detection of mutations can involve, for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, under conditions favorable for the specific annealing of these reagents to their complementary sequences within the PK gene of the invention. Preferably, the lengths of these nucleic acid reagents are at least 15, at least 25, at least 60 nucleotides, or in the range of 15-60 nucleotides. After incubation, all non-annealed nucleic acids are removed from the nucleic acid:PK gene molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microliter plate or polystyrene beads. In this case, after incubation, non-annealed, labeled nucleic acid reagents are easily removed. Detection of the remaining, annealed, labeled PK nucleic acid reagents is accomplished using standard techniques well-known to those in the art. The sequence of a PK gene to which the nucleic acid reagents have annealed can be compared to the annealing pattern expected from a sequence of a normal PK gene in order to determine whether a mutation is present.

Alternative diagnostic methods for the detection of nucleic acid molecules of a PK gene, in patient samples or other appropriate cell sources, may involve their amplification, e.g., by PCR (the experimental embodiment set forth in Mullis, K. B., 1987, U.S. Pat. No. 4,683,202), followed by the detection of the amplified molecules using techniques well known to those of skill in the art. The resulting amplified sequences can be compared to those which would be expected if the nucleic acid being amplified contained only normal copies of the PK gene in order to determine whether a mutation exists.

Among the PK nucleic acid sequences which are preferred for such hybridization and/or PCR analyses are those which will detect the presence of splice site mutation of a PK gene.

Additionally, well-known genotyping techniques can be performed to identify individuals carrying mutations in a PK gene of the invention. Such techniques include, for example, the use of restriction fragment length polymorphisms (RFLPs), which involve sequence variations in one of the recognition sites for the specific restriction enzyme used.

Additionally, improved methods for analyzing DNA polymorphisms which can be utilized for the identification of mutations in a PK gene have been described which capitalize on the presence of variable numbers of short, tandemly repeated DNA sequences between the restriction enzyme sites. For example, Weber (U.S. Pat. No. 5,075,217, which is incorporated herein by reference in its entirety) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers which are so closely spaced exhibit a high frequency co-inheritance, and are extremely useful in the identification of genetic mutations, such as, for example, mutations within a PK gene, and the diagnosis of diseases and disorders related to PK mutations.

Also, Caskey et al. (U.S. Pat. No. 5,364,759, which is incorporated herein by reference in its entirety) describe a DNA profiling assay for detecting short tri and tetra nucleotide repeat sequences. The process includes extracting the DNA of interest, such as the PK gene, amplifying the extracted DNA, and labelling the repeat sequences to form a genotypic map of the individual's DNA.

The expression level of a PK gene can also be assayed. For example, RNA from a cell type or tissue, such as a cancer cell type, may be isolated and tested utilizing hybridization or PCR techniques such as are described, below. The isolated cells can be derived from cell culture or from a patient. The analysis of cells taken from culture may be a necessary step in the assessment of cells to be used as part of a cell-based gene therapy technique or, alternatively, to test the effect of compounds on the expression of the PK gene. Such analyses may reveal both quantitative and qualitative aspects of the expression pattern of a PK gene, including activation or inactivation of the expression of a PK gene.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the PK gene nucleic acid reagents. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by utilizing any suitable nucleic acid staining method, e.g., by standard ethidium bromide staining.

Additionally, it is possible to perform such expression assays “in situ”, i.e., directly upon tissue sections (fixed and/or frozen) of patient tissue obtained from biopsies or, resections, such that no nucleic acid purification is necessary. Nucleic acids from a PK gene may be used as probes and/or primers for such in situ procedures (see, for example, Nuovo, G. J., 1992, “PCR In Situ Hybridization: Protocols And Applications”, Raven Press, NY).

Alternatively, if a sufficient quantity of the appropriate cells can be obtained, standard Northern analysis can be performed to determine the level of mRNA expression of a PK gene.

The expression of a PK gene in cells or tissues, e.g., the cellular level of PK gene transcripts and/or the presence or absence of mutations, can be evaluated using DNA microarray technologies. In such technologies, positionally addressable arrays of one or more polynucleotide probes each comprising a sequence of the PK gene are used to monitor the expression of the PK gene of the invention. The present invention therefore provides

DNA microarrays comprising polynucleotide probes comprising sequences of one or more of the PK genes.

Any formats of DNA microarray technologies can be used in conjunction with the present invention. In one embodiment, spotted cDNA arrays are prepared by depositing PCR products of cDNA fragments, e.g., full length cDNAs, ESTs, etc., of the PK gene of the invention onto a suitable surface (see, e.g., DeRisi et al., 1996, Nature Genetics 14:457-460; Shalon et al., 1996, Genome Res. 6:689-645; Schena et al., 1995, Proc. Natl. Acad. Sci. U.S.A. 93:10539-11286; and Duggan et al., Nature Genetics Supplement 21:10-14). In another embodiment, high-density oligonucleotide arrays containing oligonucleotides complementary to sequences of PK gene of the invention are synthesized in situ on the surface by photolithographic techniques (see, e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; McGall et al., 1996, Proc. Natl. Acad. Sci. U.S.A. 93:13555-13560; U.S. Pat. Nos. 5,578,832; 5,556,752; 5,510,270; 5,858,659; and 6,040,138). This format of microarray technology is particular useful for detection of single nucleotide polymorphisms (SNPs) (see, e.g., Hacia et al., 1999, Nat Genet. 22:164-7; Wang et al., 1998, Science 280:1077-82). In yet another embodiment, high-density oligonucleotide arrays containing oligonucleotides complementary to sequences of PK gene of the invention are synthesized in situ on the surface by inkjet technologies (see, e.g., Blanchard, International Patent Publication WO 98/41531, published Sep. 24, 1998; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123). In still another embodiment, DNA microarrays that allow electronic stringency control can be used in conjunction with polynucleotide probes comprising sequences of the PK gene of the invention (see, e.g., U.S. Pat. No. 5,849,486).

Quantitative reverse transcriptase PCR (qRT-PCR) can also be used to determine the expression level of a PK gene. The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™. Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system includes software for running the instrument and for analyzing the data.

5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

### 4.2.2. Detection of Abundance and Activity of a PK

Antibodies directed against wild type or mutant PKs or conserved variants or peptide fragments thereof may be used for diagnosis and prognosis of sensitivity of cancer cells to an anti-cancer agent. Such diagnostic methods may be used to detect abnormalities in the abundance level of a PK, or abnormalities in the structure and/or temporal, tissue, cellular, or subcellular location of the PK.

The tissue or cell type to be analyzed will generally include those which are known, or suspected, to express the PK gene of the invention, such as, a cancer cell type exhibiting sensitivity to anti-cancer agents. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, New York), which is incorporated herein by reference in its entirety. The isolated cells can be derived from cell culture or from a patient. The analysis of cell taken from culture may be used to test the effect of compounds on the expression of the PK gene of the invention.

Preferred diagnostic methods for the detection of PKs or conserved variants or peptide fragments thereof, may involve, for example, immunoassays wherein the PKs or conserved variants or peptide fragments are detected by their interaction with an anti-PK antibody.

For example, antibodies, or fragments of antibodies, that bind a PK of the invention, may be used to quantitatively or qualitatively detect the presence of the PK or conserved variants or peptide fragments thereof. This can be accomplished, for example, by immunofluorescence techniques employing a fluorescently labeled antibody (see below, this Section) coupled with light microscopic, flow cytometric, or fluorimetric detection. Such techniques are especially preferred if such PKs are expressed on the cell surface.

The antibodies (or fragments thereof) useful in the present invention may, additionally, be employed histologically, as in immunofluorescence or immunoelectron microscopy, for in situ detection of a PK or conserved variants or peptide fragments thereof. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the PK, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for PKs or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying a PK or a conserved variant or peptide fragment thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled PK specific antibody. The solid phase support may then be washed with the buffer a second time to remove unbound antibody. The amount of bound label on solid support may then be detected by conventional means.

By “solid phase support or carrier” as used above is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tub, or the external surface of a rod. Alternatively, the surface may be flat such as a sheet, test strip, etc.

Preferred supports include polystyrene beads. Those skilled in the art will know many other suitable carriers for binding antibody or antigen, or will be able to ascertain the same by use of routine experimentation.

The binding activity of a given lot of anti-PK antibody may be determined according to well known methods. Those skilled in the art will be able to determine operative and optimal assay conditions for each determination by employing routine experimentation.

One of the ways in which the PK gene of the invention peptide-specific antibody can be detectably labeled is by linking the same to an enzyme and use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means.

Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays.

For example, by radioactively labeling the antibodies or antibody fragments, it is possible to detect PK gene of the invention peptides through the use of a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody can also be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### 4.3. Anti-Cancer Agents

The invention can be practiced with any known anti-cancer agent, including but not limited to DNA damaging agents, anti-metabolites, anti-mitotic agents, or a combination of two or more of such known anti-cancer agents.

DNA damage agents cause chemical damage to DNA and/or RNA. DNA damage agents can disrupt DNA replication or cause the generation of nonsense DNA or RNA. DNA damaging agents include but are not limited to topoisomerase inhibitor, DNA binding agent, and ionizing radiation. A topoisomerase inhibitor that can be used in conjunction with the invention can be a topoisomerase I (Topo I) inhibitor, a topoisomerase II (Topo II) inhibitor, or a dual topoisomerase I and II inhibitor. A topo I inhibitor can be for example from any of the following classes of compounds: camptothecin analogue (e.g., karenitecin, aminocamptothecin, lurtotecan, topotecan, irinotecan, BAY 56-3722, rubitecan, GI14721, exatecan mesylate), rebeccamycin analogue, PNU 166148, rebeccamycin, TAS-103, camptothecin (e.g., camptothecin polyglutamate, camptothecin sodium), intoplicine, ecteinascidin 743, J-107088, pibenzimol. Examples of preferred topo I inhibitors include but are not limited to camptothecin, topotecan (hycaptamine), irinotecan (irinotecan hydrochloride), belotecan, or an analogue or derivative of any of the foregoing.

A topo II inhibitor that can be used in conjunction with the invention can be for example from any of the following classes of compounds: anthracycline antibiotics (e.g., carubicin, pirarubicin, daunorubicin citrate liposomal, daunomycin, 4-iodo-4-doxydoxorubicin, doxorubicin, n,n-dibenzyl daunomycin, morpholinodoxorubicin, aclacinomycin antibiotics, duborimycin, menogaril, nogalamycin, zorubicin, epirubicin, marcellomycin, detorubicin, annamycin, 7-cyanoquinocarcinol, deoxydoxorubicin, idarubicin, GPX-100, MEN-10755, valrubicin, KRN5500), epipodophyllotoxin compound (e.g., podophyllin, teniposide, etoposide, GL331, 2-ethylhydrazide), anthraquinone compound (e.g., ametantrone, bisantrene, mitoxantrone, anthraquinone), ciprofloxacin, acridine carboxamide, amonafide, anthrapyrazole antibiotics (e.g., teloxantrone, sedoxantrone trihydrochloride, piroxantrone, anthrapyrazole, losoxantrone), TAS-103, fostriecin, razoxane, XK469R, XK469, chloroquinoxaline sulfonamide, merbarone, intoplicine, elsamitrucin, CI-921, pyrazoloacridine, elliptinium, amsacrine. Examples of preferred topo II inhibitors include but are not limited to doxorubicin (Adriamycin), etoposide phosphate (etopofos), teniposide, sobuzoxane, or an analogue or derivative of any of the foregoing.

DNA binding agents that can be used in conjunction with the invention include but are not limited to a DNA groove binding agent, e.g., DNA minor groove binding agent; DNA crosslinking agent; intercalating agent; and DNA adduct forming agent. A DNA minor groove binding agent can be an anthracycline antibiotic, mitomycin antibiotic (e.g., porfiromycin, KW-2149, mitomycin B, mitomycin A, mitomycin C), chromomycin A3, carzelesin, actinomycin antibiotic (e.g., cactinomycin, dactinomycin, actinomycin F1), brostallicin, echinomycin, bizelesin, duocarmycin antibiotic (e.g., KW 2189), adozelesin, olivomycin antibiotic, plicamycin, zinostatin, distamycin, MS-247, ecteinascidin 743, amsacrine, anthramycin, and pibenzimol, or an analogue or derivative of any of the foregoing.

DNA crosslinking agents include but are not limited to antineoplastic alkylating agent, methoxsalen, mitomycin antibiotic, psoralen. An antineoplastic alkylating agent can be a nitrosourea compound (e.g., cystemustine, tauromustine, semustine, PCNU, streptozocin, SarCNU, CGP-6809, carmustine, fotemustine, methylnitrosourea, nimustine, ranimustine, ethylnitrosourea, lomustine, chlorozotocin), mustard agent (e.g., nitrogen mustard compound, such as spiromustine, trofosfamide, chlorambucil, estramustine, 2,2,2-trichlorotriethylamine, prednimustine, novembichin, phenamet, glufosfamide, peptichemio, ifosfamide, defosfamide, nitrogen mustard, phenesterin, mannomustine, cyclophosphamide, melphalan, perfosfamide, mechlorethamine oxide hydrochloride, uracil mustard, bestrabucil, DHEA mustard, tallimustine, mafosfamide, aniline mustard, chlornaphazine; sulfur mustard compound, such as bischloroethylsulfide; mustard prodrug, such as TLK286 and ZD2767), ethylenimine compound (e.g., mitomycin antibiotic, ethylenimine, uredepa, thiotepa, diaziquone, hexamethylene bisacetamide, pentamethylmelamine, altretamine, carzinophilin, triaziquone, meturedepa, benzodepa, carboquone), alkylsulfonate compound (e.g., dimethylbusulfan, Yoshi-864, improsulfan, piposulfan, treosulfan, busulfan, hepsulfam), epoxide compound (e.g., anaxirone, mitolactol, dianhydrogalactitol, teroxirone), miscellaneous alkylating agent (e.g., ipomeanol, carzelesin, methylene dimethane sulfonate, mitobronitol, bizelesin, adozelesin, piperazinedione, VNP40101M, asaley, 6-hydroxymethylacylfulvene, EO9, etoglucid, ecteinascidin 743, pipobroman), platinum compound (e.g., ZD0473, liposomal-cisplatin analogue, satraplatin, BBR 3464, spiroplatin, ormaplatin, cisplatin, oxaliplatin, carboplatin, lobaplatin, zeniplatin, iproplatin), triazene compound (e.g., imidazole mustard, CB10-277, mitozolomide, temozolomide, procarbazine, dacarbazine), picoline compound (e.g., penclomedine), or an analogue or derivative of any of the foregoing. Examples of preferred alkylating agents include but are not limited to cisplatin, dibromodulcitol, fotemustine, ifosfamide (ifosfamid), ranimustine (ranomustine), nedaplatin (latoplatin), bendamustine (bendamustine hydrochloride), eptaplatin, temozolomide (methazolastone), carboplatin, altretamine (hexamethylmelamine), prednimustine, oxaliplatin (oxalaplatinum), carmustine, thiotepa, leusulfon (busulfan), lobaplatin, cyclophosphamide, bisulfan, melphalan, and chlorambucil, or an analogue or derivative of any of the foregoing.

Intercalating agents can be an anthraquinone compound, bleomycin antibiotic, rebeccamycin analogue, acridine, acridine carboxamide, amonafide, rebeccamycin, anthrapyrazole antibiotic, echinomycin, psoralen, LU 79553, BW A773U, crisnatol mesylate, benzo(a)pyrene-7,8-diol-9,10-epoxide, acodazole, elliptinium, pixantrone, or an analogue or derivative of any of the foregoing.

DNA adduct forming agents include but are not limited to enediyne antitumor antibiotic (e.g., dynemicin A, esperamicin Al, zinostatin, dynemicin, calicheamicin gamma 1I), platinum compound, carmustine, tamoxifen (e.g., 4-hydroxy-tamoxifen), psoralen, pyrazine diazohydroxide, benzo(a)pyrene-7,8-diol-9,10-epoxide, or an analogue or derivative of any of the foregoing.

Ionizing radiation includes but is not limited to x-rays, gamma rays, and electron beams.

Anti-metabolites block the synthesis of nucleotides or deoxyribonucleotides, which are necessary for making DN, thereby preventing cells from replicating. Anti-metabolites include but are not limited to cytosine, arabinoside, floxuridine, 5-fluorouracil (5-FU), mercaptopurine, gemcitabine, hydroxyurea (HU), and methotrexate (MTX).

Anti-mitotic agents disrupt the the development of the mitotic spindle thereby interfering with tumor cell proliferation. Anti-mitotic agents include but are not limited to Vinblastine, Vincristine, and Pacitaxel (Taxol). Anti-mitotic agents also includes agents that target the enzymes that regulate mitosis, e.g., agents that target kinesin spindle protein (KSP), e.g., L-001000962-000Y.

### 4.4. Methods of Identifying Proteins or Other Molecules that Interact with a Kinase Gene and/or Its Products

Any method suitable for detecting protein-protein interactions may be employed for identifying interaction of a PK with another cellular protein. The interaction between a PK gene and other cellular molecules, e.g., interaction between PK and its regulators, may also be determined using methods known in the art. Such cellular consitituents may be the down-stream or up-stream interacting partners of the PK in a pathway in which the PK is functioning. Thus, such cellular constituents provide additional targets that can be modulated.

Among the traditional methods which may be employed are co-immunoprecipitation, crosslinking and co-purification through gradients or chromatographic columns. Utilizing procedures such as these allows for the identification of cellular proteins which interact with a PK. Once isolated, such a cellular protein can be identified and can, in turn, be used, in conjunction with standard techniques, to identify proteins it interacts with. For example, at least a portion of the amino acid sequence of the cellular protein which interacts with the PK can be ascertained using techniques well known to those of skill in the art, such as via the Edman degradation technique (see, e.g., Creighton, 1983, “Proteins: Structures and Molecular Principles”, W.H. Freeman & Co., N.Y., pp. 34-49). The amino acid sequence obtained may be used as a guide for the generation of oligonucleotide mixtures that can be used to screen for gene sequences encoding such cellular proteins. Screening may be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al., eds. Academic Press, Inc., New York).

Additionally, methods may be employed which result in the simultaneous identification of genes which encode the cellular protein interacting with a PK. These methods include, for example, probing expression libraries with labeled PK, using PK in a manner similar to the well known technique of antibody probing of λgt11 libraries.

One method which detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to a PK and the other consists of the transcription activator protein's activation domain fused to an unknown protein that is encoded by a cDNA which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites.

Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, a PK may be used as the bait protein. Total genomic or cDNA sequences are fused to the DNA encoding an activation domain. This library and a plasmid encoding a hybrid of a bait PK fused to the DNA-binding domain are cotransformed into a yeast reporter strain, and the resulting transformants are screened for those that express the reporter gene. For example, and not by way of limitation, a bait PK gene sequence, such as the coding sequence of a PK gene can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with a bait PK are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait PK-GAL4 fusion plasmid into a yeast strain which contains a lacZ gene driven by a promoter which contains GAL4 activation sequence. A cDNA encoded protein, fused to GAL4 transcriptional activation domain, that interacts with bait PK will reconstitute an active GAL4 protein and thereby drive expression of the HIS3 gene. Colonies which express HIS3 can be detected by their growth on petri dishes containing semi-solid agar based media lacking histidine. The cDNA can then be purified from these strains, and used to produce and isolate the bait PK-interacting protein using techniques routinely practiced in the art.

Genes or proteins in a cell of a cell type which interact with, e.g., modulate the effect of, another gene/protein or an agent, e.g., a drug, can also be identified using RNA interference. As used herein, interaction of a gene with an agent or another gene includes interactions of the gene and/or its products with the agent or another gene/gene product. For example, an identified gene may confer sensitivity to a drug, i.e., reduces or enhances the effect of the drug. Such gene or genes can be identified by knocking down a plurality of different genes in cells of the cell type using a plurality of small interfering RNAs (knockdown cells), each of which targets one of the plurality of different genes, and determining which gene or genes among the plurality of different genes whose knockdown modulates the response of the cell to the agent. In one embodiment, a plurality of different knockdown cells (a knockdown library) are generated, each knockdown cell in the knockdown library comprising a different gene that is knockdown, e.g., by an siRNA. In another embodiment, a plurality of different knockdown cells (a knockdown library) are generated, each knockdown cell in the knockdown library comprising 2 or more different genes that are knockdown, e.g., by shRNA and siRNA targeting different genes. In one embodiment, the knockdown library comprises a plurality of cells, each of which expresses an siRNA targeting a primary gene and is supertransfected with one or more siRNAs targeting a secondary gene. It will be apparent to one skilled in the art that a knockdown cell may also be generated by other means, e.g., by using antisense, ribozyme, antibody, or a small organic or inorganic molecule that target the gene or its product. It is envisioned that any of these other means and means utilizing siRNA can be used alone or in combination to generate a knockdown library of the invention. Any method for siRNA silencing may be used, including methods that allow tuning of the level of silencing of the target gene.

In one embodiment, the method of the invention is practiced using an siRNA knockdown library comprising a plurality of cells of a cell type each comprising one of a plurality of siRNAs, each of the plurality of siRNAs targeting and silencing (i.e., knocking down) one of a plurality of different genes in the cell (i.e., knockdown cells). Any known method of introducing siRNAs into a cell can be used for this purpose. Preferably, each of the plurality of cells is generated and maintained separately such that they can be studied separately. Each of the plurality of cells is then treated with an agent, and the effect of the agent on the cell is determined. The effect of the agent on a cell comprising a gene silenced by an siRNA is then compared with the effect of the agent on cells of the cell type which do not comprise an siRNA, i.e., normal cells of the cell type. Knockdown cell or cells which exhibit a change in response to the agent are identified. The gene which is silenced by the comprised siRNA in such a knockdown cell is a gene which modulates the effect of the agent. Preferably, the plurality of siRNAs comprises siRNAs targeting and silencing at least 5, 10, 100, or 1,000 different genes in the cells. In a preferred embodiment, the plurality of siRNAs target and silence endogenous genes.

In a preferred embodiment, the knockdown library comprises a plurality of different knockdown cells having the same gene knocked down, e.g., each cell having a different siRNA targeting and silencing a same gene. The plurality of different knockdown cells having the same gene knocked down can comprises at least 2, 3, 4, 5, 6 or 10 different knockdown cells, each of which comprises an siRNA targeting a different region of the knocked down gene. In another preferred embodiment, the knockdown library comprises a plurality of different knockdown cells, e.g., at least 2, 3, 4, 5, 6, or 10, for each of a plurality of different genes represented in the knockdown library. In still another preferred embodiment, the knockdown library comprises a plurality of different knockdown cells, e.g., at least 2, 3, 4, 5, 6, or 10, for each of all different genes represented in the knockdown library.

In another preferred embodiment, the knockdown library comprises a plurality of different knockdown cells having different genes knocked down, each of the different knockdown cells has two or more different siRNA targeting and silencing a same gene. In preferred embodiment, each different knockdown cell can comprises at least 2, 3, 4, 5, 6 or 10 different siRNAs targeting the same gene at different regions.

In a preferred embodiment, the interaction of a gene with an agent is evaluated based on responses of a plurality of different knockdown cells having the gene knocked down, e.g., each cell having a different siRNA targeting and silencing a same gene. Utilizing the responses of a plurality of different siRNAs allows determination of the on-target and off-target effect of different siRNAs (see, e.g., International Application Publication No. WO 2005/018534, published on Mar. 3, 2005).

The effect of the agent on a cell of a cell type may be reduced in a knockdown cell as compared to that of a normal cell of the cell type, i.e., the knockdown of the gene mitigates the effect of the agent. The gene which is knocked down in such a cell is said to confer sensitivity to the agent. Thus, in one embodiment, the method of the invention is used for identifying one or more genes that confer sensitivity to an agent.

The effect of the agent on a cell of a cell type may be enhanced in a knockdown cell as compared to that of a normal cell of the cell type. The gene which is knocked down in such a cell is said to confer resistance to the agent. Thus, in another embodiment, the method of the invention is used for identifying a gene or genes that confers resistance to an agent.

The enhancement of an effect of an agent may be additive or synergistic. In one embodiment, the invention provides a method for identifying one or more genes capable of enhancing the growth inhibitory effect of an anti-cancer drug in a cancer cell.

Such a method can be used for evaluating a plurality of different agents. For example, sensitivity to a plurality of different anti-cancer agents described in Section 4.3 may be evaluated by the method of the invention. In a preferred embodiment, sensitivity of each knockdown cell in the knockdown library to each of the plurality of different agents is evaluated to generate a two-dimensional responsiveness matrix comprising measurement of effect of each agent on each knockdown cell. A cut at the gene axis at a particular gene index gives a profile of responses of the particular knockdown cell (in which the particular gene is knocked down) to different drugs. A cut at the drug axis at a particular drug gives a gene responsiveness profile to the drug, i.e., a profile comprising measurements of effect of the drug on different knockdown cells in the knockdown library.

Interaction between different genes can also be identified by using an agent that modulates, e.g., suppresses or enhances, the expression of a gene and/or an activity of a protein encoded by the gene. Examples of such agents include but are not limited to siRNA, antisense, ribozyme, antibody, and small organic or inorganic molecules that target the gene or its product. The gene targeted by such an agent is termed the primary target. Such an agent can be used in conjunction with a knockdown library to identify gene or genes which modulates the response of the cell to the agent. The primary target can be different from any of the plurality of genes represented in the knockdown library (secondary genes). The gene or genes identified as modulating the effect of the agent are therefore gene or genes that interact with the primary target.

Interaction between different genes can also be identified using a dual siRNA approach. In a preferred embodiment, dual RNAi screens is achieved through the use of stable in vivo delivery of an shRNA disrupting the primary target gene and supertransfection of an siRNA targeting a secondary target gene. This approach is described in greater detail in Section 4.5., infra. In a preferred embodiment, matched cell lines (+/− primary target gene), e.g., cells containing either empty pRS vector or pRS-shRNA (see, e.g., Section 4.6., infra), are generated and used.

Silencing of the secondary target gene is then carried out using cells of a generated a cell containing an shRNA that targets a primary target gene. Silencing of the secondary target gene can be achieved using any known method of RNA interference (see, e.g., Section 4.6., International Application Publication No. WO 2005/018534, published on Mar. 3, 2005). For example, secondary target gene can be silenced by transfection with siRNA and/or plasmid encoding an shRNA. In one embodiment, cells of a generated shRNA primary target clone are supertransfected with one or more siRNAs targeting a secondary target gene. In one embodiment, the one or more siRNAs targeting the secondary gene are transfected into the cells directly. In another embodiment, the one or more siRNAs targeting the secondary gene are transfected into the cells via shRNAs using one or more suitable plasmids. RNA can be harvested 24 hours post transfection and knockdown assessed by TaqMan analysis.

In a preferred embodiment, an siRNA pool containing at least k (k=2, 3, 4, 5, 6 or 10) different siRNAs targeting the secondary target gene at different sequence regions is used to supertransfect the cells. In another preferred embodiment, an siRNA pool containing at least k (k=2, 3, 4, 5, 6 or 10) different siRNAs targeting two or more different secondary target genes is used to supertransfect the cells. In a preferred embodiment, the total siRNA concentration of the pool is about the same as the concentration of a single siRNA when used individually, e.g., 100 nM. Preferably, the total concentration of the pool of siRNAs is an optimal concentration for silencing the intended target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the composition of the pool, including the number of different siRNAs in the pool and the concentration of each different siRNA, is chosen such that the pool of siRNAs causes less than 30%, 20%, 10% or 5%, 1%, 0.1% or 0.01% of silencing of any off-target genes. In another preferred embodiment, the concentration of each different siRNA in the pool of different siRNAs is about the same. In still another preferred embodiment, the respective concentrations of different siRNAs in the pool are different from each other by less than 5%, 10%, 20% or 50%. In still another preferred embodiment, at least one siRNA in the pool of different siRNAs constitutes more than 90%, 80%, 70%, 50%, or 20% of the total siRNA concentration in the pool. In still another preferred embodiment, none of the siRNAs in the pool of different siRNAs constitutes more than 90%, 80%, 70%, 50%, or 20% of the total siRNA concentration in the pool. In other embodiments, each siRNA in the pool has an concentration that is lower than the optimal concentration when used individually. In a preferred embodiment, each different siRNA in the pool has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the pool has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene.

In one embodiment, the invention provides a method for identifying one or more genes which exhibit synthetic lethal interaction with a primary target gene. In the method, an agent that is an inhibitor of the primary target gene in the cell type is used to screen against a knockdown library. The gene or genes identified as enhancing the effect of the agent are therefore gene or genes that have synthetic lethal interaction with the primary target. In a preferred embodiment, the agent is an siRNA targeting and silencing the primary target.

The effect of an agent on the growth of cells having the primary target gene and the secondary target gene silenced can also be evaluated. In a preferred embodiment, matched cell lines (+/− primary target gene) are generated as described in Section 4.6. Both cell lines are then supertransfected with either a control siRNA (e.g., luciferase) or one or more siRNAs targeting a secondary target gene. The cell cycle profiles are examined with or without exposure to the agent. Cell cycle analysis can be carried out using standard method known in the art. In one embodiment, the supernatant from each well is combined with the cells that have been harvested by trypsinization. The mixture is then centrifuged at a suitable speed. The cells are then fixed with ice cold 70% ethanol for a suitable period of time, e.g., ˜30 minutes. Fixed cells can be washed once with PBS and resuspended, e.g., in 0.5 ml of PBS containing Propidium Iodide (10 microgram/ml) and RNase A(1 mg/ml), and incubated at a suitable temperature, e.g., 37° C., for a suitable period of time, e.g., 30 min. Flow cytometric analysis is carried out using a flow cytometer. In one embodiment, the Sub-G1 cell population is used to measure cell death. An increase of sub-G1 cell population in cells having the primary target gene and the secondary target gene silenced indicates synthetic lethality between the primary and secondary target genes in the presence of the agent.

Any suitable cell lines can be used. The cell lines can be HeLa cells, TP53-positive A549 cells or TP53-negative A549 cells. In one embodiment, matched pair of TP53 positive and negative cells were generated by stable transfection of short hairpin RNAs (shRNAs) targeting TP53. The cells were transfected using pools of siRNAs (pool of 3 siRNA per gene) at 100 nM (each siRNA at 33 nM), or with single siRNA at 100 nM. The following siRNAs were used: Luc control, ATR, MAST2, MAP3K6, TBK1, ADRBK2, CDKL2, LATS2, STK32B, STK11, DDR1, PSKH2, and NEK8 pools.

### 4.5. Methods for Screening Agents that Modulate PKs

Additional agents that modulate the expression or activity of a PK gene or encoded PK, i.e., ATR, MAST2, MAP3K6, TBK1, ADRBK2, CDKL2, LATS2, STK32B, STK11, DDR1, PSKH2, and NEK8 genes, and/or their encoded protein kinases, or modulate interaction of a PK with other proteins or molecules can be identified using a method described in this section. Such agents are useful in modulating, e.g., enhancing, the sensitivity of cells to the growth inhibitory effect of anti-cancer drugs and thus can be used for enhancing the growth inhibitory effect of anti-cancer drugs in a cell or organism.

### 4.5.1. Screening Assays

The following assays are designed to identify compounds that bind to a PK gene or its product, bind to other cellular protein(s) that interact with a PK, bind to cellular constituent(s), e.g., proteins, that are affected by a PK, or bind to compound(s) that interfere with the interaction of a PK gene or its product with other cellular proteins and to compounds which modulate the expression or activity of a PK gene (i.e., modulate the expression level of the PK gene and/or modulate the activity level of the PK). Assays may additionally be utilized which identify compounds which bind to PK regulatory sequences (e.g., promoter sequences), see e.g., Platt, K. A., 1994, J. Biol. Chem. 269:28558-28562, which is incorporated herein by reference in its entirety, which may modulate the level of PK gene expression. Compounds may include, but are not limited to, small organic molecules which are able to affect expression of a PK gene or some other gene involved in the PK pathway, or other cellular proteins. Methods for the identification of such cellular proteins are described, above, in Section 4.4. Such cellular proteins may be involved in the regulation of the growth inhibitory effect of an anti-cancer agent. Further, among these compounds are compounds which affect the level of PK gene expression and/or PK activity and which can be used in the regulation of sensitivity to the growth inhibitory effect of an anti-cancer agent.

Compounds may include, but are not limited to, peptides such as, for example, soluble peptides, including but not limited to, Ig-tailed fusion peptides, and members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, Z. et al., 1993, Cell 72:767-778), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small molecules.

Compounds identified via assays such as those described herein may be useful, for example, in modulating the biological function of the PK, and for ameliorating resistance to the growth inhibitory effect of an anti-cancer agent, and/or enhancing the growth inhibitory effect of an anti-cancer agent. Assays for testing the effectiveness of compounds are discussed, below, in Section 4.5.2.

In vitro systems may be designed to identify compounds capable of binding a PK. Compounds identified may be useful, for example, in modulating the activity of wild type and/or mutant PK, may be useful in elaborating the biological function of the PK, may be utilized in screens for identifying compounds that disrupt normal PK interactions, or may in themselves disrupt such interactions.

The principle of the assays used to identify compounds that bind to the PK involves preparing a reaction mixture of the PK and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring PK or the test substance onto a solid phase and detecting PK/test compound complexes anchored on the solid phase at the end of the reaction. In one embodiment of such a method, the PK may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for a PK or the test compound to anchor any complexes formed in solution, and a labeled antibody specific for the other component of the possible complex to detect anchored complexes.

The PK gene or PK may interact in vivo with one or more intracellular or extracellular molecules, such as proteins. Such molecules may include, but are not limited to, nucleic acid molecules and those proteins identified via methods such as those described, above, in Section 4.4. For purposes of this discussion, such molecules are referred to herein as “binding partners”. Compounds that disrupt PK binding may be useful in modulating the activity of the PK. Compounds that disrupt PK gene binding may be useful in modulating the expression of a PK gene, such as by modulating the binding of a regulator of a PK gene. Such compounds may include, but are not limited to molecules such as peptides which would be capable of gaining access to the PK.

The basic principle of the assay systems used to identify compounds that interfere with the interaction between the PK and its intracellular or extracellular binding partner or partners involves preparing a reaction mixture containing the PK, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of a PK and its binding partner. Control reaction mixtures are incubated without the test compound or with a placebo. The formation of any complexes between the PK gene and the binding partner is then detected. The formation of a complex in the control reaction, but not in the reaction mixture containing the test compound, indicates that the compound interferes with the interaction of the PK gene of the invention protein and the interactive binding partner. Additionally, complex formation within reaction mixtures containing the test compound and a normal PK, e.g., a wild-type, may also be compared to complex formation within reaction mixtures containing the test compound and a mutant PK. This comparison may be important in those cases wherein it is desirable to identify compounds that disrupt interactions of mutant but not the normal PK.

The assay for compounds that interfere with the interaction of the PKs and binding partners can be conducted in a heterogeneous or homogeneous format. Heterogeneous assays involve anchoring either the PK or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the PKs and the binding partners, e.g., by competition, can be identified by conducting the reaction in the presence of the test substance; i.e., by adding the test substance to the reaction mixture prior to or simultaneously with the PK and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the PK or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the PK or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternative embodiment of the invention, a homogeneous assay can be used. In this approach, a preformed complex of the PK and the interactive binding partner is prepared in which either the PK or its binding partner is labeled, but the signal generated by the label is quenched due to complex formation (see, e.g., U.S. Pat. No. 4,109,496 which utilizes this approach for immunoassays). The addition of a test substance that competes with and displaces one of the species from the preformed complex will result in the generation of a signal above background. In this way, test substances which disrupt PK/binding partner interaction can be identified.

In a particular embodiment, the PK can be prepared for immobilization using recombinant DNA techniques. For example, the coding region of PK gene can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with the radioactive isotope ¹²⁵I, for example, by methods routinely practiced in the art. In a heterogeneous assay, e.g., the GST-PK fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the PK gene and the interactive binding partner can be detected by measuring the amount of radioactivity that remains associated with the glutathione-agarose beads. A successful inhibition of the interaction by the test compound will result in a decrease in measured radioactivity.

Alternatively, the GST-PK fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the PK/binding partner interaction can be detected by adding the labeled antibody and measuring the radioactivity associated with the beads.

In another embodiment of the invention, these same techniques can be employed using peptide fragments that correspond to the binding domains of the PK and/or the interactive binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this section above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the binding partner is obtained, short gene segments can be engineered to express peptide fragments of the protein, which can then be tested for binding activity and purified or synthesized.

For example, and not by way of limitation, a PK can be anchored to a solid material as described in this section, above, by making a GST-PK fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-PK fusion protein and allowed to bind. After washing away unbound peptides, labeled bound material, representing the binding partner binding domain, can be eluted, purified, and analyzed for amino acid sequence by well-known methods. Peptides so identified can be produced synthetically or fused to appropriate facilitative proteins using recombinant DNA technology.

Kinase activity of a PK can be assayed in vitro using a synthetic peptide substrate of a PK of interest, e.g., a GSK-derived biotinylated peptide substrate. By way of example, the assay is as follows. The phosphopeptide product is quantitated using a Homogenous Time-Resolved Fluorescence (HTRF) assay system (Park et al., 1999, Anal. Biochem. 269:94-104). The reaction mixture contains suitable amounts of ATP, peptide substrate, and the PK. The peptide substrate has a suitable amino acid sequence and is biotinylated at the N-terminus. The kinase reaction is incubated, and then terminated with Stop/Detection Buffer and GSK3α anti-phosphoserine antibody (e.g., Cell Signaling Technologies, Beverly, Mass.; Cat #9338) labeled with europium-chelate (e.g., from Perkin Elmer, Boston, Mass.). The reaction is allowed to equilibrate, and relative fluorescent units are determined. Inhibitor compounds are assayed in the reaction described above, to determine compound IC50s. A particular compound is added to in a half-log dilution series covering a suitable range of concentrations, e.g., from 1 nM to 100 μM. Relative phospho substrate formation, read as HTRF fluorescence units, is measured over the range of compound concentrations and a titration curve generated using a four parameter sigmoidal fit. Specific compounds having IC₅₀ below a predetermined threshold value, e.g., ≦50 μM against a substrate, can be identified.

The extent of peptide phosphorylation can be determined by Homogeneous Time Resolved Fluorescence (HTRF) using a lanthanide chelate (Lance)-coupled monoclonal antibody specific for the phosphopeptide in combination with a streptavidin-linked allophycocyanin (SA-APC) fluorophore which binds to the biotin moiety on the peptide. When the Lance and APC are in proximity (i.e. bound to the same phosphopeptide molecule), a non-radiative energy transfer takes place from the Lance to the APC, followed by emission of light from APC at 665 nm. The assay can be run using various assay format, e.g., streptavidin flash plate assay, streptavidin filter plate assay.

A standard PICA assay can be used to assay the activity of protein kinase A (PICA). A standard PKC assay can be used to assay the activity of protein kinase C (PKC). The most common methods for assaying PICA or PKC activity involves measuring the transfer of 32P-labeled phosphate to a protein or peptide substrate that can be captured on phosphocellulose filters via weak electrostatic interactions.

PK kinase inhibitors can be identified using fluorescence polarization to monitor kinase activity. This assay utilizes GST-PK, peptide substrate, peptide substrate tracer, an anti-phospho monoclonal IgG, and the inhibitor compound. Reactions are incubated for a period of time and then terminated. Stopped reactions are incubated and fluorescence polarization values determined.

In a specific embodiment, a standard SPA Filtration Assay and FlashPlate® Kinase Assay can be used to measure the activity of a PK. In these assays, GST-PK, biotinylated peptide substrate, ATP, and ³³P-γ-ATP are allowed to react. After a suitable period of incubation, the reactions are terminated. In a PK SPA Filtration Assay, Peptide substrate is allowed to bind Scintilation proximity assay (SPA) beads (Amersham Biosciences), followed by filtration on a Packard GF/B Unifilter plate and washed with phosphate buffered saline. Dried plates are sealed and the amount of ³³P incorporated into the peptide substrate is determined. In a PK FlashPlate® Kinase Assay, a suitable amount of the reaction is transferred to streptavidin-coated FlashPlates® (NEN) and incubated. Plates are washed, dried, sealed and the amount of ³³P incorporated into the peptide substrate is determined.

A standard PK DELFIA® Kinase Assay can also be used. In a DELFIA® Kinase Assay, GST-PK, peptide substrate, and ATP are allowed to react. After the reactions are terminated, the biotin-peptide substrates are captured in the stopped reactions. Wells are washed and reacted with anti-phospho polyclonal antibody and europium labeled anti-rabbit-IgG. Wells are washed and europium released from the bound antibody is detected.

Other assays, such as those described in WO 04/080973, WO 02/070494, and WO 03/101444, may also be utilized to determine biological activity of the instant compounds.

### 4.5.2. Screening for Compunds that Modulate the Growth Inhibitory Effect of an Anti-Cancer Agent

Any agents that modulate, e.g., reduce, the expression of PK gene and/or the activity of a PK or interaction of a PK of the invention with its binding partner, e.g., compounds that are identified in Section 4.5.1., antibodies to a PK of the invention, and so on, can be further screened for its ability to enhance the growth inhibitory effect of an anti-cancer agent in cells. Any suitable proliferation or growth inhibition assays known in the art can be used for this purpose. In one embodiment, a candidate agent and an anti-cancer agent are applied to cells of a cell line, and a change in growth inhibitory effect on the cells is determined. Preferably, changes in growth inhibitory effect are determined using different concentrations of the candidate agent in conjunction with different concentrations of the anti-cancer agent such that one or more combinations of concentrations of the candidate agent and anti-cancer agent which cause 50% inhibition, i.e., the IC₅₀, are determined.

In a preferred embodiment, an MTT proliferation assay (see, e.g., van de Loosdrechet, et al., 1994, J. Immunol. Methods 174: 311-320; Ohno et al., 1991, J. Immunol. Methods 145:199-203; Ferrari et al., 1990, J. Immunol. Methods 131: 165-172; Alley et al., 1988, Cancer Res. 48: 589-601; Carmichael et al., 1987, Cancer Res. 47:936-942; Gerlier et al., 1986, J. Immunol. Methods 65:55-63; Mosmann, 1983, J. Immunological Methods 65:55-63) is used to screen for a candidate agent that can be used in conjunction with an anti-cancer agent to inhibit the growth of cells. The cells are treated with chosen concentrations of the candidate agent and an anti-cancer agent for 4 to 72 hours. The cells are then incubated with a suitable amount of 3-(4,5-dimethylthiazol-2-yl)-2,5-diphenyltetrazolium bromide (MTT) for 1-8 hours such that viable cells convert MTT into an intracellular deposit of insoluble formazan. After removing the excess MTT contained in the supernatant, a suitable MTT solvent, e.g., a DMSO solution, is added to dissolved the formazan. The concentration of MTT, which is proportional to the number of viable cells, is then measured by determining the optical density at 570 nm. A plurality of different concentrations of the candidate agent can be assayed to allow the determination of the concentrations of the candidate agent and the anti-cancer agent which causes 50% inhibition.

In another preferred embodiment, an alamarBlue™ Assay for cell proliferation is used to screen for one or more candidate agents that can be used to inhibit the growth of cells (see, e.g., Page et al., 1993, Int. J. Oncol. 3:473-476). An alamarBlue™ Assay for cell proliferation can also be used to screen for a candidate agent that can be used in conjunction with an anti-cancer agent to inhibit the growth of cells. An alamarBlue™ assay measures cellular respiration and uses it as a measure of the number of living cells. The internal environment of proliferating cells is more reduced than that of non-proliferating cells. For example, the ratios of NADPH/NADP, FADH/FAD, FMNH/FMN, and NADH/NAF increase during proliferation. AlamarBlue can be reduced by these metabolic intermediates and, therefore, can be used to monitor cell proliferation. The cell number of a treated sample as measured by alamarBlue can be expressed in percent relative to that of an untreated control sample. alamarBlue reduction can be measured by either absorption or fluorescence spectroscopy. In one embodiment, the alamarBlue reduction is determined by absorbance and calculated as percent reduced using the equation:

$$\% Reduced = \frac{( {ɛ}_{ox} {λ}_{2} ) ( A {λ}_{1} ) - ( {ɛ}_{ox} {λ}_{1} ) ( A {λ}_{2} )}{( {ɛ}_{red} {λ}_{1} ) ( {A}^{′} {λ}_{2} ) - ( {ɛ}_{red} {λ}_{2} ) ( {A}^{′} {λ}_{1} )} \times 100 \qquad ( 1 )$$

where:
λ₁=570 nm
λ₂=600 nm
(ε_{red} λ₁)=155,677 (Molar extinction coefficient of reduced alamarBlue at 570 nm)
(ε_{red} λ₂)=14,652 (Molar extinction coefficient of reduced alamarBlue at 600 nm)
(εₒₓ λ₁)=80,586 (Molar extinction coefficient of oxidized alamarBlue at 570 nm)
(εₒₓ λ₂)=117,216 (Molar extinction coefficient of oxidized alamarBlue at 600 nm)
(A λ₁)=Absorbance of test wells at 570 nm
(A λ₂)=Absorbance of test wells at 600 nm
(A′λ₁)=Absorbance of negative control wells which contain medium plus alamar Blue but to which no cells have been added at 570 nm.
(A′λ₂)=Absorbance of negative control wells which contain medium plus alamar Blue but to which no cells have been added at 600 nm. Preferably, the % Reduced of wells containing no cell was subtracted from the % Reduced of wells containing samples to determine the % Reduced above background.

In a specific embodiment, the alamarBlue™ assay is performed to determine whether transfection titration curves of siRNAs targeting PK gene of the inventions are changed by the presence of an anti-cancer agent of a chosen concentration, e.g., 6-200 nM of camptothecin. Cells were transfected with an siRNA targeting a PK gene of the invention. 4 hours after siRNA transfection, 100 microliter/well of DMEM/10% fetal bovine serum with or without the anti-cancer agent was added and the plates were incubated at 37° C. and 5% CO₂ for 68 hours. The medium was removed from the wells and replaced with 100 microliter/well DMEM/10% Fetal Bovine Serum (Invitrogen) containing 10% (vol/vol) alamarBlue™ reagent (Biosource International Inc., Camarillo, Calif.) and 0.001 volumes of 1M Hepes buffer tissue culture reagent (Invitrogen). The plates were incubated for 2 hours at 37° C. before they were read at 570 and 600 nm wavelengths on a SpectraMax plus plate reader (Molecular Devices, Sunnyvale, Calif.) using Softmax Pro 3.1.2 software (Molecular Devices). The percent reduced for wells transfected with a titration of an siRNA targeting a PK gene of the invention with or without an anti-cancer agent were compared to luciferase siRNA-transfected wells. The number calculated for % Reduced for 0 nM luciferase siRNA-transfected wells without the anti-cancer agent was considered to be 100%.

Inhibitor compounds can also be assayed for their ability to inhibit a PK in cells by monitoring the phosphorylation or autophosphorylation in response to an anti-cancer drug. Cells are grown in culture medium. Cells are pooled, counted, seeded into 6 well dishes at 200,000 cells per well in 2 ml media, and incubated. Serial dilution series of compounds or control are added to each well and incubated. Following the incubation period, an anti-cancer drug is added to all drug-treated cells and a control well. After incubation with the anti-cancer drug, each well is washed and Protease Inhibitor Cocktail Complete is added to each well. Lysates are then transferred to microcentrifuge tubes and frozen at −80° C. Lysates are thawed on ice and cleared by centrifugation and the supernatants are transferred to clean tubes. Samples are electorphoresed and proteins are transferred onto PVDF. Blots are then blocked and probed using an antibody against phospho-serine or phospho threonine. Bound antibody is visualized using a horseradish peroxidase conjugated secondary antibody and enhanced chemiluminescence. After stripping of the first antibody set, blots are re-probed for total PK, using a PK monoclonal antibody. The PK monoclonal is detected using a sheep anti-mouse IgG coupled to horseradish peroxidase and enhanced chemiluminescence. ECL exposed films are scanned and the intensity of specific bands is quantitated. Titrations are evaluated for, level of phosphor-Ser signal normalized to total PK and IC50 values are calculated.

Detection of phosphonucleolin in cell lysates can be carried out using biotinylated anti-nucleolin antibody and ruthenylated goat anti-mouse antibody. To each well of a 96-well plate is added biotynylated anti-nucleolin antibody and streptavidin coated paramagnetic beads, along with a suitable cell lysate. The antibodies and lysate are incubated. Next, another anti-phosphonucleolin antibody are added to each well of the lysate mix and incubated. Lastly, the ruthenylated goat anti-mouse antibody in antibody buffer is added to each well and incubated. The lysate antibody mixtures are read and EC50s for compound dependent increases in phosphor-nucleolin are determined.

WST Assay can be carried out as follows: cells are seeded to 96 well clear bottom plates at densities which provide linear growth curves for 72 hours. Cells are cultured under sterile conditions in appropriate media. Following the initial seeding of cells, cells are incubated at 37° C., 5% CO₂ from 17 to 24 hours at which time the appropriate anti-cancer agents are added at increasing concentrations to a point which is capable of causing at least 80% cell killing within 48 hours. At the same time as anti-cancer agent addition, PK inhibitor compound is added at fixed concentrations to each anti-cancer agent titration to observe enhancement of cell killing. Cell viability/cell killing under the conditions described above are determined.

Cell cycle analysis can be carried out using standard method known in the art. In one embodiment, the supernatant from each well is combined with the cells that have been harvested by trypsinization. The mixture is then centrifuged at a suitable speed. The cells are then fixed with, e.g., ice cold 70% ethanol for a suitable period of time, e.g., ˜30 minutes.

Fixed cells can be washed once with PBS and resuspended, e.g., in 0.5 ml of PBS containing Propidium Iodide (10 microgram/ml) and RNase A (1 mg/ml), and incubated at a suitable temperature, e.g., 37° C., for a suitable period of time, e.g., 30 min. Flow cytometric analysis is then carried out using a flow cytometer. In one embodiment, the Sub-G1 cell population is used as a measure of cell death. For example, the cells are said to have been sensitized to an agent if the Sub-G1 population from the sample treated with the agent is larger than the Sub-G1 population of sample not treated with the agent.

The compounds identified in the screen should include compounds that demonstrate the ability to selectively reduce the expression of a PK gene and enhance the growth inhibitory effect of an anti-cancer agent in cells. The compounds that can be screened include but are not limited to siRNA, antisense nucleic acid, ribozyme, triple helix forming nucleic acid, antibody, and polypeptide molecules, aptamers, and small molecules.

The compounds identified in the screen can also include compounds that modulate interaction of PK with other protein(s) or molecule(s). In one embodiment, the screen is conducted to identify compounds that modulate the interaction of a PK with its interaction partner. In another embodiment, the screen is conducted to identify compounds that modulate the interaction of PK gene of the invention with a transcription regulator.

In a specific embodiment, to measure functional activity of inhibitors of PKs in cells, compounds are assayed for their ability to abrogate DNA damage induced cell cycle arrest. The assay determines cell phospho-nucleolin levels as a measure of the quantity of cells entering M-phase after cell cycle arrest brought on by the anti-cancer agent, and by way of example, is carried out as follows:

Cells of a suitable cell line are seeded at a density of 5000 cells/well in RPMI640 media supplemented with 10% fetal bovine serum. After incubation for 24 hours at 37° C. at 5% CO₂, an anti-cancer drug, e.g., camptothecin, is added to a final concentration of 200 nM and incubated for 16 hours. An equal volume of a test compound serial dilution series in growth media plus 200 nM camptothecin and 332 nM nocodozole (final concentration: 50 ng/ml) is added and incubation at 37° C. is continued for 8 hours. Media is removed from the wells and 50 μL lysis buffer (20 mM HEPES, pH 7.5, 150 mM NaCl, 50 mM NaF, 1% Triton X-100, 10% Glycerol, 1× Proteinase Inhibitor Cocktail (Roche Diagnostics, Mannheim Germany), 1 μl/ml DNase I (Roche Diagnostics), 300 μM Sodium Orthovanadate, 1 μM Microcystin (Sigma, St. Louis, Mo.) added. The plate with lysis buffer is shaken for 30 min at 4° C. and frozen (−70° C.) for 20 min. Levels of phosphonucleolin in the cell lysates is measured using the IGEN Origen technology (BioVeris Corp., Gaithersburg, Md.).

Agents can also be tested in an in vivo animal model. By way of example, and not as a limitation, cells of an appropriate human tumor cell line are injected subcutaneously into the left flank of 6-10 week old female nude mice (Harlan) on day 0. The mice are randomly assigned to a vehicle, compound or combination treatment group. Daily subcutaneous administration begins on day 1 and continues for the duration of the experiment. Alternatively, the inhibitor test compound may be administered by a continuous infusion pump. Compound, compound combination or vehicle is delivered. Tumors are excised and weighed when all of the vehicle-treated animals exhibited lesions of 0.5-1.0 cm in diameter, typically 4 to 5.5 weeks after the cells were injected. The average weight of the tumors in each treatment group for each cell line is calculated.

### 4.6. Methods of Performing RNA Interference

Any method known in the art for gene silencing can be used in the present invention (see, e.g., Guo et al., 1995, Cell 81:611-620; Fire et al., 1998, Nature 391:806-811; Grant, 1999, Cell 96:303-306; Tabara et al., 1999, Cell 99:123-132; Zamore et al., 2000, Cell 101:25-33; Bass, 2000, Cell 101:235-238; Petcherski et al., 2000, Nature 405:364-368; Elbashir et al., Nature 411:494-498; Paddison et al., Proc. Natl. Acad. Sci. USA 99:1443-1448). The siRNAs targeting a gene can be designed according to methods known in the art (see, e.g., International Application Publication No. WO 2005/018534, published on Mar. 3, 2005, and Elbashir et al., 2002, Methods 26:199-213, each of which is incorporated herein by reference in its entirety).

An siRNA having only partial sequence homology to a target gene can also be used (see, e.g., International Application Publication No. WO 2005/018534, published on Mar. 3, 2005, which is incorporated herein by reference in its entirety). In one embodiment, an siRNA that comprises a sense strand contiguous nucleotide sequence of 11-18 nucleotides that is identical to a sequence of a transcript of a gene but the siRNA does not have full length homology to any sequences in the transcript is used to silence the gene. Preferably, the contiguous nucleotide sequence is in the central region of the siRNA molecules. A contiguous nucleotide sequence in the central region of an siRNA can be any continuous stretch of nucleotide sequence in the siRNA which does not begin at the 3′ end. For example, a contiguous nucleotide sequence of 11 nucleotides can be the nucleotide sequence 2-12, 3-13, 4-14, 5-15, 6-16, 7-17, 8-18, or 9-19. In preferred embodiments, the contiguous nucleotide sequence is 11-16, 11-15, 14-15, 11, 12, or 13 nucleotides in length.

In another embodiment, an siRNA that comprises a 3′ sense strand contiguous nucleotide sequence of 9-18 nucleotides which is identical to a sequence of a transcript of a gene but which siRNA does not have full length sequence identity to any contiguous sequences in the transcript is used to silence the gene. In this application, a 3′ 9-18 nucleotide sequence is a continuous stretch of nucleotides that begins at the first paired base, i.e., it does not comprise the two base 3′ overhang. Thus, when it is stated that a particular nucleotide sequence is at the 3′ end of the siRNA, the 2 base overhang is not considered. In preferred embodiments, the contiguous nucleotide sequence is 9-16, 9-15, 9-12, 11, 10, 9, or 8 nucleotides in length.

In one embodiment, in vitro siRNA transfection is carried out as follows: one day prior to transfection, 100 microliters of chosen cells, e.g., cervical cancer HeLa cells (ATCC, Cat. No. CCL-2), grown in DMEM/10% fetal bovine serum (Invitrogen, Carlsbad, Calif.) to approximately 90% confluency are seeded in a 96-well tissue culture plate (Corning, Corning, N.Y.) at 1500 cells/well. For each transfection 85 microliters of OptiMEM (Invitrogen) is mixed with 5 microliter of serially diluted siRNA (Dharma on, Denver) from a 20 micro molar stock. For each transfection 5 microliter OptiMEM is mixed with 5 microliter Oligofectamine reagent (Invitrogen) and incubated 5 minutes at room temperature. The 10 microliter OptiMEM/Oligofectamine mixture is dispensed into each tube with the OptiMEM/siRNA mixture, mixed and incubated 15-20 minutes at room temperature. 10 microliter of the transfection mixture is aliquoted into each well of the 96-well plate and incubated for 4 hours at 37° C. and 5% CO₂.

In preferred embodiments, an siRNA pool containing at least k (k=2, 3, 4, 5, 6 or 10) different siRNAs targeting the secondary target gene at different sequence regions is used to transfect the cells. In another preferred embodiment, an siRNA pool containing at least k (k=2, 3, 4, 5, 6 or 10) different siRNAs targeting two or more different target genes is used to transfect the cells.

In a preferred embodiment, the total siRNA concentration of the pool is about the same as the concentration of a single siRNA when used individually, e.g., 100 nM. Preferably, the total concentration of the pool of siRNAs is an optimal concentration for silencing the intended target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the composition of the pool, including the number of different siRNAs in the pool and the concentration of each different siRNA, is chosen such that the pool of siRNAs causes less than 30%, 20%, 10% or 5%, 1%, 0.1% or 0.01% of silencing of any off-target genes. In another preferred embodiment, the concentration of each different siRNA in the pool of different siRNAs is about the same. In still another preferred embodiment, the respective concentrations of different siRNAs in the pool are different from each other by less than 5%, 10%, 20% or 50%. In still another preferred embodiment, at least one siRNA in the pool of different siRNAs constitutes more than 90%, 80%, 70%, 50%, or 20% of the total siRNA concentration in the pool. In still another preferred embodiment, none of the siRNAs in the pool of different siRNAs constitutes more than 90%, 80%, 70%, 50%, or 20% of the total siRNA concentration in the pool. In other embodiments, each siRNA in the pool has an concentration that is lower than the optimal concentration when used individually. In a preferred embodiment, each different siRNA in the pool has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the pool has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene.

Another method for gene silencing is to introduce an shRNA, for short hairpin RNA (see, e.g., Paddison et al., 2002, Genes Dev. 16, 948-958; Brummelkamp et al., 2002, Science 296, 550-553; Sui, G. et al. 2002, Proc. Natl. Acad. Sci. USA 99, 5515-5520, all of which are incorporated by reference herein in their entirety), which can be processed in the cells into siRNA. In this method, a desired siRNA sequence is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid-based shRNAs can be expressed stably in cells, allowing long-term gene silencing in cells both in vitro and in vivo, e.g., in animals (see, McCaffrey et al. 2002, Nature 418, 38-39; Xia et al., 2002, Nat. Biotech. 20, 1006-1010; Lewis et al., 2002, Nat. Genetics 32, 107-108; Rubinson et al., 2003, Nat. Genetics 33, 401-406; Tiscornia et al., 2003, Proc. Natl. Acad. Sci. USA 100, 1844-1848, all of which are incorporated by reference herein in their entirety). Thus, in one embodiment, a plasmid-based shRNA is used.

In a preferred embodiment, shRNAs are expressed from recombinant vectors introduced either transiently or stably integrated into the genome (see, e.g., Paddison et al., 2002, Genes Dev 16:948-958; Sui et al., 2002, Proc Natl Acad Sci USA 99:5515-5520; Yu et al., 2002, Proc Natl Acad Sci USA 99:6047-6052; Miyagishi et al., 2002, Nat Biotechnol 20:497-500; Paul et al., 2002, Nat Biotechnol 20:505-508; Kwak et al., 2003, J Pharmacol Sci 93:214-217; Brummelkamp et al., 2002, Science 296:550-553; Boden et al., 2003, Nucleic Acids Res 31:5033-5038; Kawasaki et al., 2003, Nucleic Acids Res 31:700-707). The siRNA that disrupts the target gene can be expressed (via an shRNA) by any suitable vector which encodes the shRNA. The vector can also encode a marker which can be used for selecting clones in which the vector or a sufficient portion thereof is integrated in the host genome such that the shRNA is expressed. Any standard method known in the art can be used to deliver the vector into the cells. In one embodiment, cells expressing the shRNA are generated by transfecting suitable cells with a plasmid containing the vector. Cells can then be selected by the appropriate marker. Clones are then picked, and tested for knockdown. In a preferred embodiment, the expression of the shRNA is under the control of an inducible promoter such that the silencing of its target gene can be turned on when desired. Inducible expression of an siRNA is particularly useful for targeting essential genes.

In one embodiment, the expression of the shRNA is under the control of a regulated promoter that allows tuning of the silencing level of the target gene. This allows screening against cell s in which the target gene is partially knocked out. As used herein, a “regulated promoter” refers to a promoter that can be activated when an appropriate inducing agent is present. An “inducing agent” can be any molecule that can be used to activate transcription by activating the regulated promoter. An inducing agent can be, but is not limited to, a peptide or polypeptide, a hormone, or an organic small molecule. An analogue of an inducing agent, i.e., a molecule that activates the regulated promoter as the inducing agent does, can also be used. The level of activity of the regulated promoter induced by different analogues may be different, thus allowing more flexibility in tuning the activity level of the regulated promoter. The regulated promoter in the vector can be any mammalian transcription regulation system known in the art (see, e.g., Gossen et al, 1995, Science 268:1766-1769; Lucas et al, 1992, Annu. Rev. Biochem. 61:1131; Li et al., 1996, Cell 85:319-329; Saez et al., 2000, Proc. Natl. Acad. Sci. USA 97:14512-14517; and Pollock et al., 2000, Proc. Natl. Acad. Sci. USA 97:13221-13226). In preferred embodiments, the regulated promoter is regulated in a dosage and/or analogue dependent manner. In one embodiment, the level of activity of the regulated promoter is tuned to a desired level by a method comprising adjusting the concentration of the inducing agent to which the regulated promoter is responsive. The desired level of activity of the regulated promoter, as obtained by applying a particular concentration of the inducing agent, can be determined based on the desired silencing level of the target gene.

In one embodiment, a tetracycline regulated gene expression system is used (see, e.g., Gossen et al, 1995, Science 268:1766-1769; U.S. Pat. No. 6,004,941). A tet regulated system utilizes components of the tet repressor/operator/inducer system of prokaryotes to regulate gene expression in eukaryotic cells. Thus, the invention provides methods for using the tet regulatory system for regulating the expression of an shRNA linked to one or more tet operator sequences. The methods involve introducing into a cell a vector encoding a fusion protein that activates transcription. The fusion protein comprises a first polypeptide that binds to a tet operator sequence in the presence of tetracycline or a tetracycline analogue operatively linked to a second polypeptide that activates transcription in cells. By modulating the concentration of a tetracycline, or a tetracycline analogue, expression of the tet operator-linked shRNA is regulated.

In other embodiments, an ecdyson regulated gene expression system (see, e.g., Saez et al., 2000, Proc. Natl. Acad. Sci. USA 97:14512-14517), or an MMTV glucocorticoid response element regulated gene expression system (see, e.g., Lucas et al, 1992, Annu. Rev. Biochem. 61:1131) may be used to regulate the expression of the shRNA.

In one embodiment, the pRETRO-SUPER (pRS) vector which encodes a puromycin-resistance marker and drives shRNA expression from an H1 (RNA Pol III) promoter is used. The pRS-shRNA plasmid can be generated by any standard method known in the art. In one embodiment, the pRS-shRNA is deconvoluted from the library plasmid pool for a chosen gene by transforming bacteria with the pool and looking for clones containing only the plasmid of interest. Preferably, a 19 mer siRNA sequence is used along with suitable forward and reverse primers for sequence specific PCR. Plasmids are identified by sequence specific PCR, and confirmed by sequencing. Cells expressing the shRNA are generated by transfecting suitable cells with the pRS-shRNA plasmid. Cells are selected by the appropriate marker, e.g., puromycin, and maintained until colonies are evident. Clones are then picked, and tested for knockdown. In another embodiment, an shRNA is expressed by a plasmid, e.g., a pRS-shRNA. The knockdown by the pRS-shRNA plasmid, can be achieved by transfecting cells using Lipofectamine 2000 (Invitrogen).

In yet another method, siRNAs can be delivered to an organ or tissue in an animal, such a human, in vivo (see, e.g., Song et al. 2003, Nat. Medicine 9, 347-351; Sorensen et al., 2003, J. Mol. Biol. 327, 761-766; Lewis et al., 2002, Nat. Genetics 32, 107-108, all of which are incorporated by reference herein in their entirety). In this method, a solution of siRNA is injected intravenously into the animal. The siRNA can then reach an organ or tissue of interest and effectively reduce the expression of the target gene in the organ or tissue of the animal.

### 4.7. Production of PKs and PK Peptides

PKs, or peptide fragments thereof, can be prepared for use according to the present invention. For example, PKs, or peptide fragments thereof, can be used for the generation of antibodies, in diagnostic assays, for screening of inhibitors, or for the identification of other cellular gene products involved in the regulation of expression and/or activity of a PK gene.

The PKs or peptide fragments thereof, may be produced by recombinant DNA technology using techniques well known in the art. The amino acid sequences of the PKs of the invention are well-known and can be obtained from, e.g., GenBank®. Methods which are well known to those skilled in the art can be used to construct expression vectors containing

PK coding sequences and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding PK sequences may be chemically synthesized using, for example, synthesizers. See, for example, the techniques described in “Oligonucleotide Synthesis”, 1984, Gait, M. J. ed., IRL Press, Oxford, which is incorporated herein by reference in its entirety.

A variety of host-expression vector systems may be utilized to express the PK gene coding sequences. Such host-expression systems represent vehicles by which the coding sequences of interest may be produced and subsequently purified, but also represent cells which may, when transformed or transfected with the appropriate nucleotide coding sequences, exhibit the PK in situ. These include but are not limited to microorganisms such as bacteria (e.g., E. coli, B. subtilis) transformed with recombinant bacteriophage DNA, plasmid DNA or cosmid DNA expression vectors containing PK coding sequences; yeast (e.g., Saccharomyces, Pichia) transformed with recombinant yeast expression vectors containing the PK coding sequences; insect cell systems infected with recombinant virus expression vectors (e.g., baculovirus) containing the PK coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing PK coding sequences; or mammalian cell systems (e.g., COS, CHO, BHK, 293, 3T3, N2a) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the PK being expressed. For example, when a large quantity of such a protein is to be produced, for the generation of pharmaceutical compositions of PK protein or for raising antibodies to PK protein, for example, vectors which direct the expression of high levels of fusion protein products that are readily purified may be desirable. Such vectors include, but are not limited, to the E. coli expression vector pUR278 (Ruther et al., 1983, EMBO J. 2:1791), in which the PK coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The PK gene coding sequence may be cloned individually into non-essential regions (for example the polyhedrin gene) of the virus and placed under control of an AcNPV promoter (for example the polyhedrin promoter). Successful insertion of PK gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the PK gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing PK in infected hosts. (E.g., See Logan & Shenk, 1984, Proc. Natl. Acad. Sci. USA 81:3655-3659). Specific initiation signals may also be required for efficient translation of inserted PK coding sequences. These signals include the ATG initiation codon and adjacent sequences. In cases where an entire PK gene, including its own initiation codon and adjacent sequences, is inserted into the appropriate expression vector, no additional translational control signals may be needed. However, in cases where only a portion of the PK gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38.

For long-term, high-yield production of recombinant proteins, stable expression is preferred. For example, cell lines which stably express the PK may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the PK. Such engineered cell lines may be particularly useful in screening and evaluation of compounds that affect the endogenous activity of the PK.

In another embodiment, the expression characteristics of an endogenous gene (e.g., a PK gene) within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene (e.g., a PK gene) and controls, modulates, activates, or inhibits the endogenous gene. For example, endogenous PK genes which are normally “transcriptionally silent”, i.e., a PK gene which is normally not expressed, or is expressed only at very low levels in a cell line or microorganism, may be activated by inserting a regulatory element which is capable of promoting the expression of the gene product in that cell line or microorganism. Alternatively, transcriptionally silent, endogenous PK genes may be activated by insertion of a promiscuous regulatory element that works across cell types.

A heterologous regulatory element may be inserted into a stable cell line or cloned microorganism, such that it is operatively linked with and activates or inhibits expression of endogenous PK genes, using techniques, such as targeted homologous recombination, which are well known to those of skill in the art, and described e.g., in Chappel, U.S. Pat. No. 5,272,071; PCT Publication No. WO 91/06667 published May 16, 1991; Skoultchi, U.S. Pat. No. 5,981,214; and Treco et,al U.S. Pat. No. 5,968,502 and PCT Publication No. WO 94/12650 published Jun. 9, 1994. Alternatively, non-targeted, e.g. non-homologous recombination techniques may be used which are well-known to those of skill in the art and described, e.g., in PCT Publication No. WO 99/15650 published Apr. 1, 1999.

PK gene activation (or inactivation) may also be accomplished using designer transcription factors using techniques well known in the art. Briefly, a designer zinc finger protein transcription factor (ZFP-TF) is made which is specific for a regulatory region of the PK gene to be activated or inactivated. A construct encoding this designer ZFP-TF is then provided to a host cell in which the PK gene is to be controlled. The construct directs the expression of the designer ZFP-TF protein, which in turn specifically modulates the expression of the endogenous PK gene. The following references relate to various aspects of this approach in further detail: Wang & Pabo, 1999, Proc. Natl. Acad. Sci. USA 96, 9568; Berg, 1997, Nature Biotechnol. 15, 323; Greisman & Pabo, 1997, Science 275, 657; Berg & Shi, 1996, Science 271, 1081; Rebar & Pabo, 1994, Science 263, 671; Rhodes & Klug, 1993, Scientific American 269, 56; Pavletich & Pabo, 1991, Science 252, 809; Liu et al., 2001, J. Biol. Chem. 276, 11323; Zhang et al., 2000, J. Biol. Chem. 275, 33850; Beerli et al., 2000, Proc. Natl. Acad. Sci. USA 97, 1495; Kang et al., 2000, J. Biol. Chem. 275, 8742; Beerli et al., 1998, Proc. Natl. Acad. Sci. USA 95, 14628; Kim & Pabo, 1998, Proc. Natl. Acad. Sci. USA 95, 2812; Choo et al., 1997, J. Mol. Biol. 273, 525; Kim & Pabo, 1997, J. Biol. Chem. 272, 29795; Liu et al, 1997, Proc. Natl. Acad. Sci. USA 94, 5525; Kim et al, 1997, Proc. Natl. Acad. Sci. USA 94, 3616; Kikyo et al., 2000, Science 289, 2360; Robertson & Wolffe, 2000, Nature Reviews 1, 11; and Gregory, 2001, Curr. Opin. Genet. Devt. 11, 142.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺•nitriloacetic acid-agarose columns and histidine-tagged proteins are selectively eluted with imidazole-containing buffers.

In a specific embodiment, recombinant human PKs can be expressed as a fusion protein with glutathione S-transferase at the amino-terminus (GST-PK) using standard baculovirus vectors and a (Bac-to-Bac®) insect cell expression system purchased from GIBCO™ Invitrogen. Recombinant protein expressed in insect cells can be purified using glutathione sepharose (Amersham Biotech) using standard procedures described by the manufacturer.

### 4.8. Production of Anti-PK Antibodies

A PK or a fragment thereof can be used to raise antibodies which bind PK. Such antibodies include but are not limited to polyclonal, monoclonal, chimeric, single chain, Fab fragments, and an Fab expression library. In a preferred embodiment, anti PK C-terminal antibodies are raised using an appropriate C-terminal fragment of a PK, e.g., the kinase domain. Such antibodies bind the kinase domain of the PK. In another preferred embodiment, anti PK N-terminal antibodies are raised using an appropriate N-terminal fragment of a PK. The N-terminal domain of a PK are less homologous to other kinases, and therefore offered a more specific target for a particular PK.

### 4.8.1. Production of Monoclonal Anti-PK Antibodies

Antibodies can be prepared by immunizing a suitable subject with a PK or a fragment thereof as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see, e.g., U.S. Pat. No. 5,914,112, which is incorporated herein by reference in its entirety).

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of to HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay (ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103, Academic Press, 1986). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against a PK or a fragment thereof can be identified and isolated by screening a recombinant combinatorial immunoglobulin library (e.g., an antibody phage display library) with the PK or the fragment. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.)

Humanized antibodies are antibody molecules from non-human species having one or more complementarity determining regions (CDRs) from the non-human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. Nos. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323; antibodies against hepatitis B in Cole et al. (1991, Proc. Natl. Acad. Sci. USA 88:2869); as well as against viral antigens-respiratory syncitial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with a PK.

Monoclonal antibodies directed against a PK can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif., see, for example, U.S. Pat. No. 5,985,615) and Medarex, Inc. (Princeton, N.J.), can be engaged to provide human antibodies directed against a PK or a fragment thtereof using technology similar to that described above.

Completely human antibodies which recognize and bind a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

A pre-existing anti-PK antibody can be used to isolate additional antigens of the PK by standard techniques, such as affinity chromatography or immunoprecipitation for use as immunogens. Moreover, such an antibody can be used to detect the protein (e.g., in a cellular lysate or cell supernatant) in order to evaluate the abundance and pattern of expression of PK. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

### 4.8.2. Producion of Polyclonal Anti-PK Antibodies

The anti-PK antibodies can be produced by immunization of a suitable animal, such as but are not limited to mouse, rabbit, and horse.

An immunogenic preparation comprising a PK or a fragment thereof can be used to prepare antibodies by immunizing a suitable subject (e.g., rabbit, goat, mouse or other mammal). An appropriate immunogenic preparation can contain, for example, recombinantly expressed or chemically synthesized PK peptide or polypeptide. The preparation can further include an adjuvant, such as Freund's complete or incomplete adjuvant, or similar immunostimulatory agent.

A fragment of a PK suitable for use as an immunogen comprises at least a portion of the PK that is 8 amino acids, more preferably 10 amino acids and more preferably still, 15 amino acids long.

The invention also provides chimeric or fusion PK polypeptides for use as immunogens. As used herein, a “chimeric” or “fusion” PK polypeptide comprises all or part of a PK polypeptide operably linked to a heterologous polypeptide. Within the fusion PK polypeptide, the term “operably linked” is intended to indicate that the PK polypeptide and the heterologous polypeptide are fused in-frame to each other. The heterologous polypeptide can be fused to the N-terminus or C-terminus of the PK polypeptide.

One useful fusion PK polypeptide is a GST fusion PK polypeptide in which the PK polypeptide is fused to the C-terminus of GST sequences. Such fusion PK polypeptides can facilitate the purification of a recombinant PK polypeptide.

In another embodiment, the fusion PK polypeptide contains a heterologous signal sequence at its N-terminus so that the PK polypeptide can be secreted and purified to high homogeneity in order to produce high affinity antibodies. For example, the native signal sequence of an immunogen can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal and the protein A secretory signal (Pharmacia Biotech; Piscataway, N.J.).

In yet another embodiment, the fusion PK polypeptide is an immunoglobulin fusion protein in which all or part of a PK polypeptide is fused to sequences derived from a member of the immunoglobulin protein family. The immunoglobulin fusion proteins can be used as immunogens to produce antibodies directed against the PK polypetide in a subject.

Chimeric and fusion PK polypeptide can be produced by standard recombinant DNA techniques. In one embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (e.g., Ausubel et al., supra). Moreover, many expression vectors are commercially available that already encode a fusion domain (e.g., a GST polypeptide). A nucleic acid encoding an immunogen can be cloned into such an expression vector such that the fusion domain is linked in-frame to the polypeptide.

The PK immunogenic preparation is then used to immunize a suitable animal. Preferably, the animal is a specialized transgenic animal that can secrete human antibody. Non-limiting examples include transgenic mouse strains which can be used to produce a polyclonal population of antibodies directed to a specific pathogen (Fishwild et al., 1996, Nature Biotechnology 14:845-851; Mendez et al., 1997, Nature Genetics 15:146-156). In one embodiment of the invention, transgenic mice that harbor the unrearranged human immunoglobulin genes are immunized with the target immunogens. After a vigorous immune response against the immunogenic preparation has been elicited in the mice, the blood of the mice are collected and a purified preparation of human IgG molecules can be produced from the plasma or serum. Any method known in the art can be used to obtain the purified preparation of human IgG molecules, including but is not limited to affinity column chromatography using anti-human IgG antibodies bound to a suitable column matrix. Anti-human IgG antibodies can be obtained from any sources known in the art, e.g., from commercial sources such as Dako Corporation and ICN. The preparation of IgG molecules produced comprises a polyclonal population of IgG molecules that bind to the immunogen or immunogens at different degree of affinity. Preferably, a substantial fraction of the preparation contains IgG molecules specific to the immunogen or immunogens. Although polyclonal preparations of IgG molecules are described, it is understood that polyclonal preparations comprising any one type or any combination of different types of immunoglobulin molecules are also envisioned and are intended to be within the scope of the present invention.

A population of antibodies directed to a PK can be produced from a phage display library. Polyclonal antibodies can be obtained by affinity screening of a phage display library having a sufficiently large and diverse population of specificities with a PK or a fragment . thereof. Examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734. A phage display library permits selection of desired antibody or antibodies from a very large population of specificities. An additional advantage of a phage display library is that the nucleic acids encoding the selected antibodies can be obtained conveniently, thereby facilitating subsequent construction of expression vectors.

In other preferred embodiments, the population of antibodies directed to a PK or a fragment thereof is produced by a method using the whole collection of selected displayed antibodies without clonal isolation of individual members as described in U.S. Pat. No. 6,057,098, which is incorporated by reference herein in its entirety. Polyclonal antibodies are obtained by affinity screening of a phage display library having a sufficiently large repertoire of specificities with, e.g., an antigenic molecule having multiple epitopes, preferably after enrichment of displayed library members that display multiple antibodies. The nucleic acids encoding the selected display antibodies are excised and amplified using suitable PCR primers. The nucleic acids can be purified by gel electrophoresis such that the full length nucleic acids are isolated. Each of the nucleic acids is then inserted into a suitable expression vector such that a population of expression vectors having different inserts is obtained. The population of expression vectors is then expressed in a suitable host.

### 4.8.3 Production of Anit-PK Antibody-Drug Conjugates

Cancer cells can be targeted and killed using anti-PK antibody-drug conjugates that target a receptor protein tyrosine kinase. For example, an anti-PK antibody may be conjugated to a therapeutic moiety such as a cytotoxin, e.g., a cytostatic or cytocidal agent, or a radioactive metal ion. Antibody-drug conjugates can be prepared by method known in the art (see, e.g., Immunoconjugates, Vogel, ed. 1987; Targeted Drugs, Goldberg, ed. 1983; Antibody Mediated Delivery Systems, Rodwell, ed. 1988). Therapeutic drugs, such as but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof, can be conjugated to anti-PK antibodies of the invention. Other therapeutic agents that can be conjugated to anti-PK antibodies of the invention include, but are not limited to, antimetabolites, e.g., methotrexate, 6-mercaptopurine, 6-thioguanine, cytarabine, 5-fluorouracil decarbazine; alkylating agents, e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin; anthracyclines, e.g., daunorubicin (daunomycin) and doxorubicin; antibiotics, e.g., dactinomycin (actinomycin), bleomycin, mithramycin, anthramycin (AMC); and anti-mitotic agents, e.g., vincristine and vinblastine. The therapeutic agents that can be conjugated to anti-PK antibodies of the invention may also be a protein or polypeptide possessing a desired biological activity. Other anti-cancer agents described in Section 4.3. can also be conjugated with such an anti-PK antibody. Such proteins may include, for example, a toxin such as abrin, ricin A, pseudomonas exotoxin, or diphtheria toxin.

The drug molecules can be linked to the anti-PK antibody via a linker. Any suitable linker can be used for the preparation of such conjugates. In some embodiments, the linker can be a linker that allows the drug molecules to be released from the conjugates in unmodified form at the target site.

The antibodies can also be used diagnostically to, for example, monitor the presence of cancer cells as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See generally U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include fluorescent proteins, e.g., green fluorescent protein (GFP), umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ¹¹¹In, ¹⁷⁷Lu, ⁹⁰Y or ⁹⁹Tc.

Techniques for conjugating therapeutic moieties to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982); each of which is incorporated herein by reference.

Alternatively, an antibody can be conjugated to a second antibody to form an antibody heteroconjugate as described by Segal in U.S. Pat. No. 4,676,980, which is incorporated herein by reference.

### 4.8.4 Production of Peptides

A PK-binding peptide or polypeptide of the invention may be produced by recombinant DNA technology using techniques well known in the art. Thus, PK-binding polypeptides and peptides of the invention can be produced by expressing nucleic acid containing sequences encoding the PK-binding polypeptide or peptide. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding a PK-binding polypeptide may be chemically synthesized using, for example, synthesizers. See, for example, the techniques described in “Oligonucleotide Synthesis”, 1984, Gait, M. J. ed., IRL Press, Oxford, which is incorporated herein by reference in its entirety.

### 4.9. Kits

The invention also provides a kit for determining sensitivity of a cell to the growth inhibitory effect of an anti-cancer agent, comprising in one or more containers one or more polynucleotide probes, wherein each said polynucleotide probe comprises a nucleotide sequence complementary and hybridizable to a sequence in a gene encoding a protein kinase selected from the group consisting of ATR, MAST2, MAP3K6, TBK1, ADRBK2, CDKL2, LATS2, STK32B, STK11, DDR1, PSKH2, and NEK8; wherein said first polynucleotide probes are at least 50%, 75%, 80%, 85%, 90%, 95%, 97%, 98%, 99% or 100% of the total polynucleotide probes in said kit.

The invention also provides a kit for screening for agents which enhance sensitivity of a cell to the growth inhibitory effect of an anti-cancer agent, comprising in one or separate containers (i) a protein kinase selected from the group consisting of ATR, MAST2, MAP3K6, TBK1, ADRBK2, CDKL2, LATS2, STK32B, STK11, DDR1, PSKH2, and NEK8, or a peptide fragment thereof; and (ii) said anti-cancer agent.

The invention also provides a kit for treating a mammal having a cancer, comprising in one or more containers (i) a first agent that reduces the expression of a gene encoding a protein kinase selected from the group consisting of ATR, MAST2, MAP3K6, TBK1, ADRBK2, CDKL2, LATS2, STK32B, STK11, DDR1, PSKH2, and NEK8, and/or the activity of said protein kinase; and (ii) a therapeutically effective amount of an anti-cancer agent different from said first agent. In one embodiment, in the kit of the invention said anti-cancer agent is selected from the group consisting of a topoisomerase I inhibitor, a topoisomerase II inhibitor, a DNA binding agent, anti-metabolite, anti-mitotic agent, and ionizing radiation. In another embodiment, in the kit of the invention, said anti-cancer agent is selected from the group consisting of camptothecin, cisplatin, gemcitabine, hydoxyurea, bleomycin, L-001000962-000Y, and 5-fluorouracil.

### 4.10. Pharmaceutical Formulations and Routes of Administration

The agent that can be used to reduce the expression of the PK genes of the invention or the activity of their gene products can be administered to a patient at therapeutically effective doses of the agent to enhance the effect of chemotherapy. A therapeutically effective dose refers to that amount of the agent sufficient to result in enhancement of the growth inhibitory effect of an anti-cancer agent in cells.

### 4.10.1. Effective Dose

Toxicity and therapeutic efficacy of such agent can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### 4.10.2. Formulations and Use

Pharmaceutical compositions for use in accordance with the present invention may be formulated in conventional manner using one or more pharmaceutically acceptable carriers or excipients.

Thus, the compounds and their pharmaceutically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### 4.10.3. Routes of Administration

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic mariner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

### 4.10.4. Packing

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition. Suitable conditions indicated on the label may include treatment of a disease such as one characterized by aberrant or excessive expression or activity of a PK of the invention.

### 5. EXAMPLES

The following examples are presented by way of illustration of the present invention, and are not intended to limit the present invention in any way.

### 5.1. Example 1

### Kinases that Sensitize Cells to Anti-Cancer Drugs

This example describes screens in which kinase targets that sensitize cells to cancer chemotherapeutics were identified. In this example, results of screens using cisplatin (cis), gemcitabine (Gem), hydoxyurea (HU), bleomycin (bleo), L-001000962-000Y, camptothecin, and 5-fluorouracil (5-FU) are described. Bleomycin is a DNA intercalating agent. Cisplatin is DNA crosslinking agent. Gemcitabine is a deoxycytidine analogue whose active metabolite, dFdCTP, blocks DNA elongation and has a cytotoxic effect. 5-fluorouracil interferes with the synthesis of nucleic acids, thereby preventing cells from making DNA and RNA. Hydroxyurea is an S-phase specific inhibitor of ribonucleotide reductase (RR) with a broad spectrum of antitumor effects. L-001000962-000Y affects the mitotic spindle.

The screens were carried out using HeLa cells, HCT116-p53sh cells, or A549-p53sh cells. The cells were transfected using pools of siRNAs (pool of 3 siRNA per gene) at 100 nM (each siRNA at 33 nM), or with a single siRNA at 100 nM for HeLa and A549 and at 50 nM for HCT116 cells. siRNAs targeting ATR, MAST2, MAP3K6, TBK1, ADRBK2, CDKL2, LATS2, STK32B, STK11, DDR1, PSKH2, and NEK8 were employed. The sequences of the siRNAs used are listed in Table 2. These siRNAs were transfected into cells in the presence or absence of varying concentrations of an anti-cancer drug. The concentration for each agent was as follows: cisplatin (high 200 or 400 ng/ml, low 50 or 100 ng/ml); gemcitabine (7 and 12 nM), hydoxyurea (180 uM), bleomycin (200 ng/ml), L-001000962-000Y (60 nM), and 5-FU (1.5 nM).

siRNA transfection was carried out as follows: one day prior to transfection, 50 microliters of media containing cells of a chosen cell line, e.g., cervical cancer HeLa cells (ATCC, Cat. No. CCL-2), grown in DMEM/10% fetal bovine serum (Invitrogen, Carlsbad, Calif.), were seeded in a 384-well tissue culture plate at 250-600 cells/well. For each transfection, 18 microliters of OptiMEM (Invitrogen) were mixed with 2 microliters of siRNA (Proligo, Boulder, Colo.) from a 10 micromolar stock. For each transfection, 20 microliters of OptiMEM were mixed with 1 microliter of Oligofectamine reagent (Invitrogen) (Hela) or 0.3 microliter SilentFect (BioRad) (A549) or 0.5 microliter Lipofectamine 2000 (Invitrogen) (HCT116) and incubated for 5 minutes at room temperature. Then the 20-microliter OptiMEM/Transfection reagent mixture was mixed with the 20-microliter of OptiMEM/siRNA mixture, and incubated for 15-20 minutes at room temperature. 5 microliters (HeLa and A549) or 2.5 microliters (HCT116) of the transfection mixture were aliquoted into each well of the 384-well plate and incubated for 4 hours at 37° C. and 5% CO₂.

After 4 hours, 5 microliters/well of DMEM/10% fetal bovine serum with or without an anti-cancer agent were added to each well to reach the final concentration of each agents as described above. The plates were incubated at 37° C. and 5% CO₂ for another 68 or 92 hours. Samples were then assessed for the number of viable cells by an Alamar Blue Assay. For the Alamar assay, the media were aspirated and then 25 microliters of DMEM/10% FBS +10 Alamar Blue reagent (BioSource) were added to each well of the 384 well plate. Fluorescence was measured using a microplate reader at 1-2 hrs post reagent addition. Cell growth was calculated as % viability relative to a control siRNA (i.e. viability relative to wells transfected with an siRNA to Luciferase). Fold sensitization was calculated by dividing the percent viability in the absence of drug by the percent viability in the presence of drug.

TABLE 2
siRNA sequences used in the kinase screens

| | Human | | Human | | | |
|---|---|---|---|---|---|---|
| Gene | Reference | | Reference | | | |
| Name | Transcript | SEQ ID NO | Protein | SEQ ID NO | siRNA probes-sense sequence | SEQ ID NO |
| ATR | NM_001184 | SEQ ID NO 1 | NP_001175 | SEQ ID NO 2 | GCCAGUGUAUGCUACCAAA | SEQ ID NO 3 |
| | | | | | CCGCUAAUCUUCUAACAUU | SEQ ID NO 4 |
| | | | | | GCUUAAGUCUGAUUUGCUA | SEQ ID NO 5 |
| MAST2 | NM_015112 | SEQ ID NO 6 | NP_055927 | SEQ ID NO 7 | GUAUGAGGGUCAUAUUGAA | SEQ ID NO 8 |
| | | | | | CCGCCAGAAGGCUGAAUUU | SEQ ID NO 9 |
| | | | | | CAGUCUGCACCUUCUCUUA | SEQ ID NO 10 |
| MAP3K6 | NM_004672 | SEQ ID NO 11 | NP_004663 | SEQ ID NO 12 | GCACUUUGAGGAUUCCAAA | SEQ ID NO 13 |
| | | | | | CGCCCGAUCUGUACUGCAU | SEQ ID NO 14 |
| | | | | | GAGAUGUUGGAGUUUGAUU | SEQ ID NO 15 |
| TBK1 | NM_013254 | SEQ ID NO 16 | NP_037386 | SEQ ID NO 17 | GCGGCAGAGUUAGGUGAAA | SEQ ID NO 18 |
| | | | | | CCACAAAUUUGAUAAGCAA | SEQ ID NO 19 |
| | | | | | CUCUGAAUACCAUAGGAUU | SEQ ID NO 20 |
| ADRBK2 | NM_005160 | SEQ ID NO 21 | NP_005151 | SEQ ID NO 22 | GCAAUGAAAUGCUUAGAUA | SEQ ID NO 23 |
| | | | | | GUCCUUUCAUUGUAUGUAU | SEQ ID NO 24 |
| | | | | | GCUGCUGAUGCCUUUGAUA | SEQ ID NO 25 |
| CDKL2 | NM_003948 | SEQ ID NO 26 | NP_003939 | SEQ ID NO 27 | GUGAUGAAGUGUAGGAAUA | SEQ ID NO 28 |
| | | | | | GCCAUUGGUUGUCUGGUAA | SEQ ID NO 29 |
| | | | | | GUUACAGAGUGGAUGAGAA | SEQ ID NO 30 |
| LATS2 | NM_014572 | SEQ ID NO 31 | NP_055387 | SEQ ID NO 32 | CCAUGAAGACCCUAAGGAA | SEQ ID NO 33 |
| | | | | | GACCUUAUCAGAAAGCCUU | SEQ ID NO 34 |
| | | | | | GCCUCAACGUGGACCUGUA | SEQ ID NO 35 |
| STK32B | NM_018401 | SEQ ID NO 36 | NP_060871 | SEQ ID NO 37 | GGGAGGAAUUCAUCAUAUU | SEQ ID NO 38 |
| | | | | | GAAGUCAACUUUGACCAUU | SEQ ID NO 39 |
| | | | | | GCCCUACAUGGCUCCAGAA | SEQ ID NO 40 |
| STK11 | NM_000455 | SEQ ID NO 41 | NP_000446 | SEQ ID NO 42 | CCGUCAAGAUCCUCAAGAA | SEQ ID NO 43 |
| | | | | | GCCAACGUGAAGAAGGAAA | SEQ ID NO 44 |
| | | | | | CGCAGCAGCUGGGCAUGUU | SEQ ID NO 45 |
| DDR1 | NM_013994 | SEQ ID NO 46 | NP_054700 | SEQ ID NO 47 | GGAGCUACCGGCUGCGUUA | SEQ ID NO 48 |
| | | | | | GGGAUGGACUCCUGUCUUA | SEQ ID NO 49 |
| | | | | | CAGCUUCUCCAGUGGCUAU | SEQ ID NO 50 |
| PSKH2 | NM_033126 | SEQ ID NO 51 | NP_149117 | SEQ ID NO 52 | GGAUCAAGUUUACAUGGUA | SEQ ID NO 53 |
| | | | | | GACUGAGGAUCAAGUUUAC | SEQ ID NO 54 |
| | | | | | GCGGGUUAGCCAUCGUUAC | SEQ ID NO 55 |
| NEK8 | NM_178170 | SEQ ID NO 56 | NP_835464 | SEQ ID NO 57 | GGCUUUGCUGGGCUAUGAA | SEQ ID NO 58 |
| | | | | | CCCAGAACAUCCUGCUUGA | SEQ ID NO 59 |
| | | | | | GCGGCACUCUGGCUGAGUU | SEQ ID NO 60 |

TABLE 3A
results of the screens. The results listed in the table represent the fold sensitization
for four HeLa cisplatin experiments with each experiment run at a low and high dose of
cisplatin. The number in a parenthese is the experiment number.

| | | | Hela cis | | | | Hela cis | Hela cis | Hela cis |
|---|---|---|---|---|---|---|---|---|---|
| | Translated | Hela cis | high | Hela cis | Hela cis | Hela cis | high | low | high |
| Kinase | Identifier | low (29) | (29) | low (3) | high (3) | low (78) | (78) | (100) | (100) |
| ATR | NM_001184 | | 4.41 | | | | 3.78 | 3.23 | 3.52 |
| MAST2 | NM_015112 | | | | | | | | |
| MAP3K6 | NM_004672 | | | | | | 2.95 | | |
| TBK1 | NM_013254 | | | | | | | | |
| ADRBK2 | NM_005160 | | | | | | 3.24 | | |
| CDKL2 | NM_003948 | | | | 2.66 | | | 2.89 | 3.06 |
| LATS2 | NM_014572 | | | | | | | 2.42 | 2.55 |
| STK32B | NM_018401 | | | | | | | | |
| STK11 | NM_000455 | | | | | | | | |
| DDR1 | NM_013994 | | | | | 2.15 | 4.20 | | |
| PSKH2 | NM_033126 | | | | | | 2.63 | 2.65 | 2.93 |
| NEK8 | NM_178170 | 2.85 | | | 2.97 | | | 3.04 | |

TABLE 3B
results for HCT116 p53sh cells and A549 p53sh cells at different doses of
cisplatin. The results listed in the table represent the fold sensitization.

| | | | | HCT116 | | |
|---|---|---|---|---|---|---|
| | Translated | A549 | A549 | p53sh | HCT116 p53sh | HCT116 p53sh |
| Kinase | Identifier | 750 ng | 1500 ng | 62 ng | 125 ng | 500 ng |
| ATR | NM_001184 | 3.13 | 4.89 | | 3.31 | 7.26 |
| MAST2 | NM_015112 | | 2.03 | | | |
| MAP3K6 | NM_004672 | 2.11 | | | | 5.19 |
| TBK1 | NM_013254 | | 2.32 | | | |
| ADRBK2 | NM_005160 | | | | | |
| CDKL2 | NM_003948 | | | | | 3.72 |
| LATS2 | NM_014572 | | | | | |
| STK32B | NM_018401 | | | | | |
| STK11 | NM_000455 | | | | | |
| DDR1 | NM_013994 | | | | | |
| PSKH2 | NM_033126 | | | | | |
| NEK8 | NM_178170 | | | | 4.12 | 8.17 |

TABLE 3C
results for HeLa cells treated with different anti-cancer drugs. The results listed in
the table represent the fold sensitization

| | | | | | | L- | |
|---|---|---|---|---|---|---|---|
| | | | gemc ID77 | gemc ID77 | HU ID93 | 001000962- | |
| Kinase | 5-FU | bleo | high | low | High | 000Y | campto |
| ATR | | 4.26 | | | 3.50 | | |
| MAST2 | | 3.85 | | | 2.92 | | |
| MAP3K6 | | 6.17 | | | | | |
| TBK1 | | | | | | | |
| ADRBK2 | 3.10 | 4.34 | | | | | |
| CDKL2 | | | 2.83 | 2.20 | | 4.02 | |
| LATS2 | | | | 2.28 | 3.00 | 9.95 | |
| STK32B | 2.64 | | | | | | |
| STK11 | | | | | | | |
| DDR1 | | | | | | 3.35 | 3.90 |
| PSKH2 | | | | | | | |
| NEK8 | | | | | | | |

### 5.2. Example 2

### In Vitro Kinase Inhibitor Screening Assays

Kinase activity of each PK is assayed in vitro using a synthetic peptide substrate of the PK of interest. The phosphopeptide product is quantitated using a Homogenous Time-Resolved Fluorescence (HTRF) assay system (Park et al., 1999, Anal. Biochem. 269:94-104). The reaction mixture contains 40 mM HEPES, pH 7.3; 100 mM NaCl; 10 mM MgCl₂; 2 mM dithiothreitol; 0.1% BSA; 0.1 mM ATP; 0.5 μM peptide substrate; and 0.1 nM PK in a final volume of 40 μl. The peptide substrate has a suitable amino acid sequence and is biotinylated at the N-terminus. The kinase reaction is incubated for 30 minutes at 22° C., and then terminated with 60 μl Stop/Detection Buffer (40 mM HEPES, pH 7.3; 10 mM EDTA; 0.125% Triton X-100; 1.25% BSA; 250 nM PhycoLink Streptavidin-Allophycocyanin (APC) Conjugate (Prozyme, San Leandro, Calif.); and 0.75 nM GSK3α anti-phosphoserine antibody (Cell Signaling Technologies, Beverly, Mass.; Cat #9338) labeled with europium-chelate (Perkin Elmer, Boston, Mass.). The reaction is allowed to equilibrate for 2 hours at 22° C., and relative fluorescent units are read on a Discovery plate reader (Packard Biosciences). Inhibitor compounds are assayed in the reaction described above, to determine compound IC50s. 1 μL of compound dissolved in DMSO is added to each 40 μL reaction in a half-log dilution series covering a range of 1 nM to 100 μM. Relative phospho substrate formation, read as HTRF fluorescence units, is measured over the range of compound concentrations and a titration curve generated using a four parameter sigmoidal fit. Specific compounds of the instant invention are tested in the assay described above, and compounds are identified if they are found to have IC₅₀ of ≦50 μM against substrate.

Activated PKs are assayed utilizing a GSK-derived biotinylated peptide substrate. The extent of peptide phosphorylation is determined by Homogeneous Time Resolved Fluorescence (HTRF) using a lanthanide chelate(Lance)-coupled monoclonal antibody specific for the phosphopeptide in combination with a streptavidin-linked allophycocyanin (SA-APC) fluorophore which will bind to the biotin moiety on the peptide. When the Lance and APC are in proximity (i.e. bound to the same phosphopeptide molecule), a non-radiative energy transfer takes place from the Lance to the APC, followed by emission of light from APC at 665 nm.

Materials required for the assay:

A. Activated PK

B. PK peptide substrate: GSK3α (S21) peptide

C. Lance labeled anti-phospho GSK3α monoclonal antibody (Cell Signaling Technology, clone #27).

D. SA-APC (Prozyme catalog no. PJ25S lot #896067).

E. Microfluor® B U Bottom Microtiter Plates (Dynex Technologies, Catalog no. 7205).

F. Discovery® HTRF Microplate Analyzer, Packard Instrument Company.

G. 100 X Protease Inhibitor Cocktail (PIC): 1 mg/ml benzamidine, 0.5 mg/ml pepstatin, 0.5 mg/ml leupeptin, 0.5 mg/ml aprotinin.

H. 10× Assay Buffer: 500 mM HEPES, pH 7.5, 1% PEG, mM EDTA, 1 mM EGTA, 1% BSA, 20 mM θ-Glycerol phosphate.

I. Quench Buffer: 50 mM HEPES pH 7.3, 16.6 mM EDTA, 0.1% BSA, 0.1% Triton X-100, 0.17 nM Lance labeled monoclonal antibody clone #27, 0.0067 mg/ml SA-APC

J. ATP/MgCl₂ working solution: 1× Assay buffer, 1 mM DTT, 1× PIC, 125 mM KCl, 5% Glycerol, 25 mM MgCl₂, 375 TM ATP

K. Enzyme working solution: 1× Assay buffer, 1 mM DTT, 1× PIC, 5% Glycerol, active Akt. The final enzyme concentrations were selected so that the assay was in a linear response range.

L. Peptide working solution: 1× Assay buffer, 1 mM DTT, 1× PIC, 5% Glycerol, 2 TM GSK3 biotinylated peptide #3928

The reaction is assembled by adding 16 μl of the ATP/MgCl₂ working solution to the appropriate wells of a 96-well microtiter plate. Inhibitor or vehicle (1.0 μl) is added followed by 10 μl of peptide working solution. The reaction is started by adding 13 μl of the enzyme working solution and mixing. The reaction is allowed to proceed for 50 min and then stopped by the addition of 60 μl HTRF quench buffer. The stopped reactions are incubated at room temperature for at least 30 min and then are read on the Discovery instrument.

Procedure for Streptavidin Flash Plate Assay:

Step 1:

A 1 μl solution of the test compound in 100% DMSO is added to 20 μl of 2× substrate solution (20 uM GSK3 Peptide, 300 μM ATP, 20 mM MgCl₂, 20 μCi/ml [γ³³P] ATP, 1× Assay Buffer, 5% glycerol, 1 mM DTT, 1× PIC, 0.1% BSA and 100 mM KCl). Phosphorylation reactions are initiated by adding 19 μl of 2× Enzyme solution (6.4 nM active Akt/PKB, 1× Assay Buffer, 5% glycerol, 1 mM DTT, 1× PIC and 0.1% BSA). The reactions are then incubated at room temperature for 45 minutes.

Step 2:

The reaction is stopped by adding 170 μl of 125 mM EDTA. 200 μl of stopped reaction was transferred to a Streptavidin Flashplate® PLUS (NEN Life Sciences, catalog no. SMP103). The plate is incubated for ≧10 minutes at room temperature on a plate shaker. The contents of each well is aspirated, and the wells rinsed 2 times with 200 μl TBS per well. The wells are then washed 3 times for 5 minutes with 200 μl TBS per well with the plates incubated at room temperature on a platform shaker during wash steps.

The plates are covered with sealing tape and counted using the Packard TopCount with the appropriate settings for counting [³³P] in Flashplates.

Procedure for Streptavidin Filter Plate Assay:

Step 1:

The enzymatic reactions as described in Step 1 of the Streptavidin Flash Plate Assay above are performed.

Step 2:

The reaction is stopped by adding 20 μl of 7.5M Guanidine Hydrochloride. 50 μl of the stopped reaction is transferred to the Streptavidin filter plate (SAM²™ Biotin Capture Plate, Promega, catalog no. V7542) and the reaction is incubated on the filter for 1-2 minutes before applying vacuum.

The plate is then washed using a vacuum manifold as follows: 1) 4×200 μl/well of 2M NaCl; 2) 6×200 μl/well of 2M NaCl with 1% H₃PO₄; 3) 2×200 μl/well of diH₂0; and 4) 2×100 μl/well of 95% Ethanol. The membranes are then allowed to air dry completely before adding scintillant.

The bottom of the plate is sealed with white backing tape, 30 μl/well of Microscint 20 (Packard Instruments, catalog no. 6013621) is added. The top of the plate is sealed with clear sealing tape, and the plate then counted using the Packard TopCount with the appropriate settings for [³³P] with liquid scintillant.

Procedure for Phosphocellulose Filter Plate Assay:

Step 1:

The enzymatic reactions are performed as described in Step 1 of the Streptavidin Flash Plate Assay (above) but utilizing a non-biotinylated substracte.

Step 2:

The reaction is stopped by adding 20 μl of 0.75% H₃PO₄. 50 μl of stopped reaction is transferred to the filter plate (UNIFILTER™, Whatman P81 Strong Cation Exchanger, White Polystyrene 96 Well Plates, Polyfiltronics, catalog no. 7700-3312) and the reaction incubated on the filter for 1-2 minutes before applying vacuum.

The plate is then washed using a vacuum manifold as follows: 1) 9×200 μl/well of 0.75% H₃PO₄; and 2) 2×200 μl/well of diH₂0. The bottom of the plate is sealed with white backing tape, then 30 μl/well of Microscint 20 was added. The top of the plate is sealed with clear sealing tape, and the plate counted using the Packard TopCount with the appropriate settings for [³³P] and liquid scintillant.

PKA Assay:

Each individual PKA assay consists of the following components:

A. 5× PKA assay buffer (200 mM Tris pH7.5, 100 mM MgCl₂, 5 mM θ-mercaptoethanol, 0.5 mM EDTA)

B. 50 μM stock of Kemptide (Sigma) diluted in water

C. ³³P-ATP prepared by diluting 1.0 μl ³³P-ATP [10 mCi/ml] into 200 μl of a 50 μM stock of unlabeled ATP

D. 10 μl of a 70 nM stock of PKA catalytic subunit (UBI catalog #14-114) diluted in 0.5 mg/ml BSA

E. PKA/Kemptide working solution: equal volumes of 5× PKA assay buffer, Kemptide solution and PKA catalytic subunit.

The reaction is assembled in a 96 deep-well assay plate. The inhibitor or vehicle (10 Tl) is added to 10 μl of the ³³P-ATP solution. The reaction is initiated by adding 30 μl of the PKA/Kemptide working solution to each well. The reactions are mixed and incubated at room temperature for 20 min. The reactions are stopped by adding 50 μl of 100 mM EDTA and 100 mM sodium pyrophosphate and mixing.

The enzyme reaction product (phosphorylated Kemptide) is collected on p81 phosphocellulose 96 well filter plates (Millipore). To prepare the plate, each well of a p81 filter plate is filled with 75 mM phosphoric acid. The wells are emptied through the filter by applying a vacuum to the bottom of the plate. Phosphoric acid (75 mM, 170 μl) is added to each well. A 30 μl aliquot from each stopped PKA reaction is added to corresponding wells on the filter plate containing the phosphoric acid. The peptide is trapped on the filter following the application of a vacuum and the filters were washed 5 times with 75 mM phosphoric acid. After the final wash, the filters are allowed to air dry. Scintillation fluid (30 μl) is added to each well and the filters counted on a TopCount (Packard).

PKC Assay:

Each PKC assay consists of the following components:

A. 10× PKC co-activation buffer: 2.5 mM EGTA, 4 mM CaCl₂

B. 5× PKC activation buffer: 1.6 mg/ml phosphatidylserine, 0.16 mg/ml diacylglycerol, 100 mM Tris pH 7.5, 50 mM MgCl₂, 5 mM θ-mercaptoethanol

C. ³³P-ATP prepared by diluting 1.0 μl ³³P-ATP [10 mCi/ml] into 100 μl of a 100 μM stock of unlabeled ATP

D. Myelin basic protein (350 μg/ml, UBI) diluted in water

E. PKC (50 ng/ml, UBI catalog #14-115) diluted into 0.5 mg/ml BSA

F. PKC/Myelin Basic Protein working solution: is prepared by mixing 5 volumes each of PKC co-activation buffer and Myelin Basic protein with 10 volumes each of PKC activation buffer and PKC.

The assays are assembled in 96 deep-well assay plates Inhibitor or vehicle (10 μl) is added to 5.0 ul of ³³P-ATP. Reactions are initiated with the addition of the PKC/Myelin Basic Protein working solution and mixing. Reactions are incubated at 30° C. for 20 min. The reactions are stopped by adding 50 μl of 100 mM EDTA and 100 mM sodium pyrophosphate and mixing. Phosphorylated Mylein Basic Protein is collected on PVDF membranes in 96 well filter plates and quantitated by scintillation counting.

Specific compounds of the instant invention are tested in the assay described above, and compounds are identified if they are found to have IC₅₀ of ≦50 μM against substrate.

PK Expression and Purification: Recombinant human PKs can be expressed as a fusion protein with glutathione S-transferase at the amino-terminus (GST-PK) using standard baculovirus vectors and a (Bac-to-Bac®) insect cell expression system purchased from GIBCO™ Invitrogen. Recombinant protein expressed in insect cells can be purified using glutathione sepharose (Amersham Biotech) using standard procedures described by the manufacturer.

PK Fluorescense Polarization Assays: PK kinase inhibitors can be identified using fluorescence polarization to monitor kinase activity. This assay utilizes 10 nM GST-PK and contains 5 mM 2-(N-Morpholino)ethanesulfonic acid (MES, pH 6.5), 5 mM magnesium chloride (MgCl₂), 0.05% Tween®-20, 1 μM adenosine 5′ triphosphate (ATP), 2 mM 1,4-Dithio-DL-threitol (DTT), 1 μM peptide substrate, 10 nM peptide substrate tracer, 60 ng anti-phospho-CREB(S133) mouse monoclonal IgG purified on Protein G sepharose from crude mouse ascites purchased from Cell Signalling Technologies (Beverly, Mass.), 4% dimethyl sulfoxide (DMSO) and 30 μM inhibitor compound. Reactions are incubated at room temperature for 140 minutes and terminated by addition of 25 mM EDTA (pH 8.0). Stopped reactions are incubated for 120 minutes at room temperature and fluorescence polarization values determined using a Molecular Devices/LJL Biosystems Analyst™ AD (Sunnyvale, Calif.) with standard fluorescine settings.

PK SPA Filtration Assay: Assays (25 μl) contain 10 nM GST-PK, 10 mM MES, 2 mM DTT, 10 mM MgCl₂, 0.025% Tween®-20, 1 uM biotinylated peptide substrate, 1 μM ATP, 0.1 μCi ³³P-γ-ATP (New England Nuclear, NEN) and are reacted for 90 minutes at room temperature. Reactions are terminated by adding 55 μl of phosphate buffered saline containing 50 mM EDTA, 6.9 mM ATP, 0.5 mg Scintilation proximity assay (SPA) beads (Amersham Biosciences). Peptide substrate is allowed to bind beads for 10 minutes at room temperature followed by filtration on a Packard GF/B Unifilter plate and washed with phosphate buffered saline. Dried plates are sealed with Topseal™ (NEN) and the amount of ³³P incorporated into the peptide substrate is determined using a Packard Topcount® scintillation counter with standard settings for ³³P.

PK FlashPlate® Kinase Assay: Assays (25 μl) contain 8.7 GST-PK, 10 mM MES, 0.1 mM ethylene glycol-bis(β-aminoethylether)-N,N,N′,N′-tetracetic acid (EGTA, pH 8.0), 2 mM DTT, 0.05% Tween 20, 3 μM peptide substrate (Biotin-ILSRRPSYRKILND-free acid) (SEQ ID NO: 19), 1 μM ATP, 0.4 μCi 33P-γ-ATP (NEN) and 4% DMSO. Reactions are incubated for 30 minutes at room temperature, terminated with 50 μl of 50 mM EDTA. 90 μl of reaction is transferred to streptavidin-coated FlashPlates® (NEN) and incubated for 1 hour at room temperature. Plates are washed with phosphate buffered saline containing 0.01% Tween-20 and 10 mM sodium pyrophosphate. Plates are dried, sealed with Topseal™ (NEN) and an amount of ³³P incorporated into the peptide substrate is measured using a Packard Topcount® NXT™ scintillation counter with standard settings.

PK DELFIA® Kinase Assay: Assays (25 μl) utilize 6.4 mM GST-PK containing 25 mM Tris, pH 8.5, 20% glycerol, 50 mM sodium chloride (NaCl), 0.1 Surfact-Amps® 20, 1 μM peptide substrate, 2 mM DTT, 4% DMSO, 12.5 μM ATP, 5 mM MgCl₂ and are reacted for 30 minutes at room temperature. Reactions are terminated with 100 μl Stop buffer containing 1% BSA, 10 mM Tris, pH 8.0, 150 mM NaCl and 100 mM EDTA. Stopped reactions (100 μl) are transferred to 96 well neutravidin plates (Pierce) to capture the biotin-peptide substrate during a 30 minute room temperature incubation. Wells are washed and reacted with 100 μl PerkinElmer Wallac Assay Buffer containing 21.5 ng/ml anti-phospho-Ser216-Cdc25c rabbit polyclonal antibody from Cell Signalling Technology (Beverly, Mass.) and 292 ng/ml europium labeled anti-rabbit-IgG for 1 hour at room temperature. Wells are washed and europium is released from the bound antibody by addition of Enhancement Solution (100 μl) (PerkinElmer Wallac) and detected using a Wallac Victor2™ using standard manufacturer settings.

Compounds of the present invention may be tested in the PK FlashPlate® Kinase Assay described above.

WST Assay: cells are seeded (75 μl ) to 96 well clear bottom plates at densities which provide linear growth curves for 72 hours. Cells are cultured under sterile conditions in appropriate media and for HT29 and HCT116 this media is McCoy's 5A containing 10% Fetal Bovine Serum (FBS). Following the initial seeding of cells, cells are incubated at 37° C., 5% CO₂ from 17 to 24 hours at which time the appropriate anti-cancer agents (camptothicins, 5-fluorouracil and etoposide) are added at increasing concentrations to a point which is capable of causing at least 80% cell killing within 48 hours. The final volume of all anti-cancer agent and compound additions is 25 Assays contain <1% DMSO final. At the same time as anti-cancer agent addition, PK inhibitor compound is added at fixed concentrations to each anti-cancer agent titration to observe enhancement of cell killing. Cell viability/cell killing under the conditions described above is determined by addition of WST reagent (Roche) according to the manufacturer at 47 hours following DNA damage and PK inhibitor compound addition and following a 3.5 hour or 2.5 hour incubation at 37° C., 5% CO₂ wherein OD₄₅₀ is measured.

Compounds of the present invention may be tested in the assays described above.

### 5.3. Example 3

### Cells Based Kinase Inhibitor Screening Assays

Inhibitor compounds are assayed for their ability to inhibit a PK in cells by monitoring the phosphorylation or autophosphorylation in response to DNA damage. Cells are grown in culture medium: RPMI 1640 supplemented with 10% fetal bovine serum; 10 mM HEPES; 2 mM L-glutamine; 1× non-essential amino acids; and penicillin-streptomycin. Cells from T-75 flasks are pooled, counted, seeded into 6 well dishes at 200,000 cells per well in 2 ml media, and incubated. Serial dilution series of compounds in DMSO or DMSO control are added to each well from a 1000× working stock in DMSO and incubated for 2 hr at 37° C. Following the 2-hr incubation period, 100 nM camptothecin (EMD Biosciences, San Diego, Calif.) is added from a 200× working stock in PBS to all drug-treated cells (except one of the high dose wells) and one DMSO control well. After a 4 hour incubation with camptothecin, each well is washed once with ice-cold PBS and 300 μL of lysis buffer (50 mM Tris (pH 8.0), 150 mM NaCl, 50 mM NaF, 1% NP-40, 0.5% Deoxycholic acid, 0.1% SDS, 0.5 μM Na₃VO₄ and 1× Protease Inhibitor Cocktail Complete—without EDTA (Roche Diagnostics, Mannheim, Germany)) is added to each well. Plates are shaken at 4° C. for 10-15 min and lysates are then transferred to 1.5 ml microcentrifuge tubes and frozen at −80° C. Lysates are thawed on ice and cleared by centrifugation at 15,000×g for 20 min and the supernatants are transferred to clean tubes.

Samples (20 μL) are prepared for gel electrophoresis by addition of 5 μL of 5× sample loading buffer and heat-denaturation for 5 min at 100° C. Samples are electorphoresed in Tris/Glycine SDS-polyacrylamide gels (10%) and proteins are transferred onto PVDF. Blots are then blocked for 1 hr in 3% BSA in TBS and probed using an antibody against phospho-serine or phospho threonine. Bound antibody is visualized using a horseradish peroxidase conjugated secondary antibody (goat anti-rabbit Jackson Labs—Cat #111-035-046) and enhanced chemiluminescence (ECL-plus, Amersham, Piscataway, N.J.). After stripping of the first antibody set by incubation in 62.5 mM Tris HCl pH 6.7, 2% SDS and 2-mercaptoethanol to 100 μM for 30 min at 55° C., blots are re-probed for total PK, using a PK monoclonal antibody (Santa Cruz Biotechnology Inc., Cat #SC-8408). The PK monoclonal is detected using a a sheep anti-mouse IgG coupled to horseradish peroxidase (Amersham Biosciences, Piscataway, N.J., Cat #NA931) and enhanced chemiluminescence (ECL-plus, Amersham). ECL exposed films are scanned and the intensity of specific bands is quantitated with ImageQuant software. Titrations are evaluated for level of phospho-(Ser) signal normalized to total PK and IC50 values are calculated.

Detection of Phosphonucleolin in Cell Lysates

Anti-nucleolin antibody (Research Diagnostics Inc., Flanders, N.J.) is biotinylated using Origen Biotin-LC-NHS-Ester (BioVeris Corp.) using the protocol described by the manufacturer. Goat anti-mouse antibody (Jackson Immuno Research, West Grove, Pa.) is ruthenylated employing a ruthenylation kit (BioVeris Corp.; cat #110034) according to the protocol described by the manufacturer. To each well of a 96-well plate is added 25 μL of antibody buffer (phospho buffered saline pH7.2, 1% bovine serum albumin, 0.5% Tween-20) containing 2 μg/ml biotynylated the anti-nucleolin antibody and 0.4 mg/ml streptavidin coated paramagnetic Dynabeads (BioVeris Corp.) along with 25 μL of cell lysate (above). The antibodies and lysate are incubated with shaking for 1 hr at room temperature. Next, 50 ng of an anti-phosphonucleolin antibody (Applied NeuroSolutions Inc., Vernon Hills, Ill.) in a volume of 50 μL of antibody buffer (above) are added to each well of the lysate mix and incubation is continued for 30 min at room temperature. Lastly, 25 μl of a 240 ng/ml solution of the ruthenylated goat anti-mouse antibody in antibody buffer is added to each well and incubation continued for 3 hours at room temperature. The lysate antibody mixtures are read in a BioVeris M-series M8 analyser and EC50s for compound dependent increases in phosphor-nucleolin are determined.

### 5.4. Example 4

### Checkpoint Escape Assay DNA Damage Arrest

To measure functional activity of inhibitors of PKs in cells, compounds are assayed for their ability to abrogate DNA damage induced cell cycle arrest. The assay determines cell phospho-nucleolin levels as a measure of the quantity of cells entering M-phase after cell cycle arrest brought on by an anti-cancer agent, e.g., camptothecin.

H1299 cells (ATCC, Manassas Va.) are seeded at a density of 5000 cells/well in RPMI640 media supplemented with 10% fetal bovine serum. After incubation for 24 hours at 37° C. at 5% CO₂, the anti-cancer agent is added to a final concentration of 200 nM and incubated for 16 hours. An equal volume of a test compound serial dilution series in growth media plus 200 nM camptothecin and 332 nM nocodozole (final concentration: 50 ng/ml) is added and incubation at 37° C. is continued for 8 hours. Media is removed from the wells and 50 μL lysis buffer (20 mM HEPES, pH7.5, 150 mM NaCl, 50 mM NaF, 1% Triton X-100, 10% Glycerol, 1× Proteinase Inhibitor Cocktail (Roche Diagnostics, Mannheim Germany), 1 μl/ml DNase I (Roche Diagnostics), 300 μM Sodium Orthovanadate, 1 μM Microcystin (Sigma, St. Louis, Mo.) added. The plate with lysis buffer is shaken for 30 min at 4° C. and frozen (−70° C.) for 20 min. Levels of phosphonucleolin in the cell lysates is measured using the IGEN Origen technology (BioVeris Corp., Gaithersburg, Md.).

### 5.5. Example 5

### In Vivo Efficacy of an Inhibitor of the Growth of Cancer Cells

Human tumor cell lines are injected subcutaneously into the left flank of 6-10 week old female nude mice (Harlan) on day 0. The mice are randomly assigned to a vehicle, compound or combination treatment group. Daily subcutaneous administration begins on day 1 and continues for the duration of the experiment. Alternatively, the inhibitor test compound may be administered by a continuous infusion pump. Compound, compound combination or vehicle is delivered in a total volume of 0.2 ml. Tumors are excised and weighed when all of the vehicle-treated animals exhibited lesions of 0.5-1.0 cm in diameter, typically 4 to 5.5 weeks after the cells were injected. The average weight of the tumors in each treatment group for each cell line is calculated.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. (canceled)

2. A method for treating a mammal having a cancer, comprising
(a) administering to said mammal a therapeutically effective amount of a first agent, said first agent reducing the expression of a gene encoding a protein kinase selected from the group consisting of ATR, MAST2, MAP3K6, TBK1, ADRBK2, CDKL2, LATS2, STK32B, STK11, DDR1, PSKH2, and NEK8 and/or activity of said protein kinase; and
(b) administering to said mammal a therapeutically effective amount of a composition comprising one or more anti-cancer agents.

3. The method of claim 2, wherein said first agent comprises a substance selected from the group consisting of siRNA, antisense nucleic acid, ribozyme, and triple helix forming nucleic acid, each being capable of reducing the expression of said gene in cells of said cancer.

4. The method of claim 3, wherein said first agent comprises an siRNA targeting said gene.

5. The method of claim 4, wherein said first agent comprises 2, 3, 4, 5, 6, or 10 different siRNAs targeting said gene.

6. The method of claim 2, wherein said first agent comprises a substance selected from the group consisting of antibody, peptide, and small molecule, each being capable of reducing the activity of said protein kinase in cells of said cancer.

7. The method of claim 2, wherein said one or more anti-cancer agents are selected from the group consisting of topoisomerase I inhibitor, topoisomerase II inhibitor, DNA binding agent, anti-metabolite, anti-mitotic agent, and ionizing radiation.

8. The method of claim 7, wherein said one or more anti-cancer agents are selected from the group consisting of camptothecin, cisplatin, gemcitabine, hydoxyurea, bleomycin, L-001000962-000Y, and 5-fluorouracil.

9. (canceled)

10. (canceled)

11. (canceled)

12. (canceled)

13. (canceled)

14. (canceled)

15. (canceled)

16. (canceled)

17. (canceled)

18. A method for evaluating sensitivity of a cell to the growth inhibitory effect of an anti-cancer agent, said method comprising determining a level of activity of a protein kinase selected from the group consisting of ATR, MAST2, MAP3K6, TBK1, ADRBK2, CDKL2, LATS2, STK32B, STK11, DDR1, PSKH2, and NEK8, and determining whether said level of activity is below a predetermined threshold level, wherein said activity level below the predetermined threshold level indicates that said cell is sensitive to the growth inhibitory effect of said anti-cancer agent.

19. The method of claim 18, wherein said anti-cancer agent is selected from the group consisting of topoisomerase I inhibitor, topoisomerase II inhibitor, DNA binding agent, anti-metabolite, anti-mitotic agent, and ionizing radiation.

20. The method of claim 19, wherein said anti-cancer agent is selected from the group consisting of camptothecin, cisplatin, gemcitabine, hydoxyurea, bleomycin, L-001000962-000Y, and 5-fluorouracil.

21. The method of claim 18, wherein said cell is a human cell.

22. A method for enhancing sensitivity of a cell to an anti-cancer agent, comprising contacting said cell with an agent that reduces the expression of a gene encoding a protein kinase selected from the group consisting of ATR, MAST2, MAP3K6, TBK1, ADRBK2, CDKL2, LATS2, STK32B, STK11, DDR1, PSKH2, and NEK8 and/or the activity of said protein kinase, said agent being in an amount sufficient to enhance sensitivity of said cell to an anti-cancer agent.

23. The method of claim 22, wherein said agent comprises a substance selected from the group consisting of siRNA, antisense nucleic acid, ribozyme, and triple helix forming nucleic acid.

24. The method of claim 22, wherein said agent comprises a substance selected from the group consisting of antibody, peptide, and small molecule.

25. The method of claim 23, wherein said anti-cancer agent is selected from the group consisting of topoisomerase I inhibitor, topoisomerase II inhibitor, DNA binding agent, anti-metabolite, anti-mitotic agent, and ionizing radiation.

26. The method of claim 23, wherein said anti-cancer agent is selected from the group consisting of camptothecin, cisplatin, gemcitabine, hydoxyurea, bleomycin, L-001000962-000Y, and 5-fluorouracil.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A method of identifying a substance that is capable of enhancing sensitivity of a cell of a cell type to the growth inhibitory effect of an anti-cancer agent, wherein said substance is capable of reducing the expression of a first gene encoding a protein kinase selected from the group consisting of ATR, MAST2, MAP3K6, TBK1, ADRBK2, CDKL2, LATS2, STK32B, STK11, DDR1, PSKH2, and NEK8 and/or the activity of said protein kinase, said method comprising:
(a) contacting a first cell of said cell type expressing said first gene with said anti-cancer agent in the presence of said candidate substance and measuring a first growth inhibitory effect;
(b) contacting a second cell of said cell type expressing said first gene with said anti-cancer agent under the same conditions as (a) except in the absence of said candidate substance and measuring a second growth inhibitory effect; and
(c) comparing said first and second growth inhibitory effects measured in said step (a) and (b),
wherein a greater first growth inhibitory effect than said second growth inhibitory effect identifies said candidate substance as capable of enhancing sensitivity of a cell to the growth inhibitory effect of said anti-cancer agent.

41. (canceled)

42. (canceled)

43. The method of claim 40, wherein said substance comprises a molecule that reduces expression of said gene.

44. The method of claim 43, wherein said substance comprises an siRNA targeting said gene.

45. The method of claim 44, wherein said substance comprises 2, 3, 4, 5, 6, or 10 different siRNAs targeting said gene.

46. The method of claim 45, wherein the total siRNA concentration of said different siRNAs in said substance is an optimal concentration for silencing said gene, wherein said optimal concentration is a concentration further increase of which does not increase the level of silencing substantially.

47. (canceled)

48. (canceled)

49. (canceled)

50. (canceled)

51. (canceled)

52. (canceled)

53. The method of claim 40, wherein said anti-cancer agent is selected from the group consisting of a topoisomerase I inhibitor, a topoisomerase II inhibitor, a DNA binding agent, anti-metabolite, anti-mitotic agent, and ionizing radiation.

54. The method of claim 53, wherein said anti-cancer agent is selected from the group consisting of camptothecin, cisplatin, gemcitabine, hydoxyurea, bleomycin, L-001000962-000Y, and 5-fluorouracil.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)


SEQUENCE LISTING (60 sequences)

SEQ ID NO: 1 - DNA, 7935 bp, Homo sapiens
atgggggaacatggcctggagctggcttccatgatccccgccctgcgggagctgggcagtgccacaccagaggaatataatacagttgtacagaagccaagacaaattctgtgtcaattcattgaccggatacttacagatgtaaatgttgttgctgtagaacttgtaaagaaaactgactctcagccaacctccgtgatgttgcttgatttcatccagcatatcatgaaatcctccccacttatgtttgtaaatgtgagtggaagccatgagcgcaaaggcagttgtattgaattcagtaattggatcataacgagacttctgcggattgcagcaactccctcctgtcatttgttacacaagaaaatctgtgaagtcatctgttcattattatttctttttaaaagcaagagtcctgctatttttggggtactcacaaaagaattattacaactttttgaagacttggtttacctccatagaagaaatgtgatgggtcatgctgtggaatggccagtggtcatgagccgatttttaagtcaattagatgaacacatgggatatttacaatcagctcctttgcagttgatgagtatgcaaaatttagaatttattgaagtcactttattaatggttcttactcgtattattgcaattgtgttttttagaaggcaagaactcttactttggcagataggttgtgttctgctagagtatggtagtccaaaaattaaatccctagcaattagctttttaacagaactttttcagcttggaggactaccagcacaaccagctagcacttttttcagctcatttttggaattattaaaacaccttgtagaaatggatactgaccaattgaaactctatgaagagccattatcaaagctgataaagacactatttccctttgaagcagaagcttatagaaatattgaacctgtctatttaaatatgctgctggaaaaactctgtgtcatgtttgaagacggtgtgctcatgcggcttaagtctgatttgctaaaagcagctttgtgccatttactgcagtatttccttaaatttgtgccagctgggtatgaatctgctttacaagtcaggaaggtctatgtgagaaatatttgtaaagctcttttggatgtgcttggaattgaggtagatgcagagtacttgttgggcccactttatgcagctttgaaaatggaaagtatggaaatcattgaggagattcaatgccaaactcaacaggaaaacctcagcagtaatagtgatggaatatcacccaaaaggcgtcgtctcagctcgtctctaaacccttctaaaagagcaccaaaacagactgaggaaattaaacatgtggacatgaaccaaaagagcatattatggagtgcactgaaacagaaagctgaatcccttcagatttcccttgaatacagtggcctaaagaatcctgttattgagatgttagaaggaattgctgttgtcttacaactgactgctctgtgtactgttcattgttctcatcaaaacatgaactgccgtactttcaaggactgtcaacataaatccaagaagaaaccttctgtagtgataacttggatgtcattggatttttacacaaaagtgcttaagagctgtagaagtttgttagaatctgttcagaaactggacctggaggcaaccattgataaggtggtgaaaatttatgatgctttgatttatatgcaagtaaacagttcatttgaagatcatatcctggaagatttatgtggtatgctctcacttccatggatttattcccattctgatgatggctgtttaaagttgaccacatttgccgctaatcttctaacattaagctgtaggatttcagatagctattcaccacaggcacaatcacgatgtgtgtttcttctgactctgtttccaagaagaatattccttgagtggagaacagcagtttacaactgggccctgcagagctcccatgaagtaatccgggctagttgtgttagtggattttttatcttattgcagcagcagaattcttgtaacagagttcccaagattcttatagataaagtcaaagatgattctgacattgtcaagaaagaatttgcttctatacttggtcaacttgtctgtactcttcacggcatgttttatctgacaagttctttaacagaacctttctctgaacacggacatgtggacctcttctgtaggaacttgaaagccacttctcaacatgaatgttcatcttctcaactaaaagcttctgtctgcaagccattccttttcctactgaaaaaaaaaatacctagtccagtaaaacttgctttcatagataatctacatcatctttgtaagcatcttgattttagagaagatgaaacagatgtaaaagcagttcttggaactttattaaatttaatggaagatccagacaaagatgttagagtggcttttagtggaaatatcaagcacatattggaatccttggactctgaagatggatttataaaggagctttttgtcttaagaatgaaggaagcatatacacatgcccaaatatcaagaaataatgagctgaaggataccttgattcttacaacaggggatattggaagggccgcaaaaggagatttggtaccatttgcactcttacacttattgcattgtttgttatccaagtcagcatctgtctctggagcagcatacacagaaattagagctctggttgcagctaaaagtgttaaactgcaaagttttttcagccagtataagaaacccatctgtcagtttttggtagaatcccttcactctagtcagatgacagcacttccgaatactccatgccagaatgctgacgtgcgaaaacaagatgtggctcaccagagagaaatggctttaaatacgttgtctgaaattgccaacgttttcgactttcctgatcttaatcgttttcttactaggacattacaagttctactacctgatcttgctgccaaagcaagccctgcagcttctgctctcattcgaactttaggaaaacaattaaatgtcaatcgtagagagattttaataaacaacttcaaatatattttttctcatttggtctgttcttgttccaaagatgaattagaacgtgcccttcattatctgaagaatgaaacagaaattgaactggggagcctgttgagacaagatttccaaggattgcataatgaattattgctgcgtattggagaacactatcaacaggtttttaatggtttgtcaatacttgcctcatttgcatccagtgatgatccatatcagggcccgagagatatcatatcacctgaactgatggctgattatttacaacccaaattgttgggcattttggctttttttaacatgcagttactgagctctagtgttggcattgaagataagaaaatggccttgaacagtttgatgtctttgatgaagttaatgggacccaaacatgtcagttctgtgagggtgaagatgatgaccacactgagaactggccttcgattcaaggatgattttcctgaattgtgttgcagagcttgggactgctttgttcgctgcctggatcatgcttgtctgggctcccttctcagtcatgtaatagtagctttgttacctcttatacacatccagcctaaagaaactgcagctatcttccactacctcataattgaaaacagggatgctgtgcaagattttcttcatgaaatatattttttacctgatcatccagaattaaaaaagataaaagccgttctccaggaatacagaaaggagacctctgagagcactgatcttcagacaactcttcagctctctatgaaggccattcaacatgaaaatgtcgatgttcgtattcatgctcttacaagcttgaaggaaaccttgtataaaaatcaggaaaaactgataaagtatgcaacagacagtgaaacagtagaacctattatctcacagttggtgacagtgcttttgaaaggttgccaagatgcaaactctcaagctcggttgctctgtggggaatgtttaggggaattgggggcgatagatccaggtcgattagatttctcaacaactgaaactcaaggaaaagattttacatttgtgactggagtagaagattcaagctttgcctatggattattgatggagctaacaagagcttaccttgcgtatgctgataatagccgagctcaagattcagctgcctatgccattcaggagttgctttctatttatgactgtagagagatggagaccaacggcccaggtcaccaattgtggaggagatttcctgagcatgttcgggaaatactagaacctcatctaaataccagatacaagagttctcagaagtcaaccgattggtctggagtaaagaagccaatttacttaagtaaattgggtagtaactttgcagaatggtcagcatcttgggcaggttatcttattacaaaggttcgacatgatcttgccagtaaaattttcacctgctgtagcattatgatgaagcatgatttcaaagtgaccatctatcttcttccacatattctggtgtatgtcttactgggttgtaatcaagaagatcagcaggaggtttatgcagaaattatggcagttctaaagcatgacgatcagcataccataaatacccaagacattgcatctgatctgtgtcaactcagtacacagactgtgttctccatgcttgaccatctcacacagtgggcaaggcacaaatttcaggcactgaaagctgagaaatgtccacacagcaaatcaaacagaaataaggtagactcaatggtatctactgtggattatgaagactatcagagtgtaacccgttttctagacctcataccccaggatactctggcagtagcttcctttcgctccaaagcatacacacgagctgtaatgcactttgaatcatttattacagaaaagaagcaaaatattcaggaacatcttggatttttacagaaattgtatgctgctatgcatgaacctgatggagtggccggagtcagtgcaattagaaaggcagaaccatctctaaaagaacagatccttgaacatgaaagccttggcttgctgagggatgccactgcttgttatgacagggctattcagctagaaccagaccagatcattcattatcatggtgtagtaaagtccatgttaggtcttggtcagctgtctactgttatcactcaggtgaatggagtgcatgctaacaggtccgagtggacagatgaattaaacacgtacagagtggaagcagcttggaaattgtcacagtgggatttggtggaaaactatttggcagcagatggaaaatctacaacatggagtgtcagactgggacagctattattatcagccaaaaaaagagatatcacagctttttatgactcactgaaactagtgagagcagaacaaattgtacctctttcagctgcaagctttgaaagaggctcctaccaacgaggatatgaatatattgtgagattgcacatgttatgtgagttggagcatagcatcaaaccacttttccagcattctccaggtgacagttctcaagaagattctctaaactgggtagctcgactagaaatgacccagaattcctacagagccaaggagcctatcctggctctccggagggctttactaagcctcaacaaaagaccagattacaatgaaatggttggagaatgctggctgcagagtgccagggtagctagaaaggctggtcaccaccagacagcctacaatgctctccttaatgcaggggaatcacgactcgctgaactgtacgtggaaagggcaaagtggctctggtccaagggtgatgttcaccaggcactaattgttcttcaaaaaggtgttgaattatgttttcctgaaaatgaaaccccacctgagggtaagaacatgttaatccatggtcgagctatgctactagtgggccgatttatggaagaaacagctaactttgaaagcaatgcaattatgaaaaaatataaggatgtgaccgcgtgcctgccagaatgggaggatgggcatttttaccttgccaagtactatgacaaattgatgcccatggtcacagacaacaaaatggaaaagcaaggtgatctcatccggtatatagttcttcattttggcagatctctacaatatggaaatcagttcatatatcagtcaatgccacgaatgttaactctatggcttgattatggtacaaaggcatatgaatgggaaaaagctggccgctccgatcgtgtacaaatgaggaatgatttgggtaaaataaacaaggttatcacagagcatacaaactatttagctccatatcaatttttgactgctttttcacaattgatctctcgaatttgtcattctcacgatgaagtttttgttgtcttgatggaaataatagccaaagtatttctagcctatcctcaacaagcaatgtggatgatgacagctgtgtcaaagtcatcttatcccatgcgtgtgaacagatgcaaggaaatcctcaataaagctattcatatgaaaaaatccttagagaagtttgttggagatgcaactcgcctaacagataagcttctagaattgtgcaataaaccggttgatggaagtagttccacattaagcatgagcactcattttaaaatgcttaaaaagctggtagaagaagcaacatttagtgaaatcctcattcctctacaatcagtcatgatacctacacttccatcaattctgggtacccatgctaaccatgctagccatgaaccatttcctggacattgggcctatattgcagggtttgatgatatggtggaaattcttgcttctcttcagaaaccaaagaagatttctttaaaaggctcagatggaaagttctacatcatgatgtgtaagccaaaagatgacctgagaaaggattgtagactaatggaattcaattccttgattaataagtgcttaagaaaagatgcagagtctcgtagaagagaacttcatattcgaacatatgcagttattccactaaatgatgaatgtgggattattgaatgggtgaacaacactgctggtttgagacctattctgaccaaactatataaagaaaagggagtgtatatgacaggaaaagaacttcgccagtgtatgctaccaaagtcagcagctttatctgaaaaactcaaagtattccgagaatttctcctgcccaggcatcctcctatttttcatgagtggtttctgagaacattccctgatcctacatcatggtacagtagtagatcagcttactgccgttccactgcagtaatgtcaatggttggttatattctggggcttggagaccgtcatggtgaaaatattctctttgattctttgactggtgaatgcgtacatgtagatttcaattgtcttttcaataagggagaaacctttgaagttccagaaattgtgccatttcgcctgactcataatatggttaatggaatgggtcctatgggaacagagggtctttttcgaagagcatgtgaagttacaatgaggctgatgcgtgatcagcgagagcctttaatgagtgtcttaaagacttttctacatgatcctcttgtggaatggagtaaaccagtgaaagggcattccaaagcgccactgaatgaaactggagaagttgtcaatgaaaaggccaagacccatgttcttgacattgagcagcgactacaaggtgtaatcaagactcgaaatagagtgacaggactgccgttatctattgaaggacatgtgcattaccttatacaagaagctactgatgaaaacttactatgccagatgtatcttggttggactccatatatgtga

SEQ ID NO: 2 - protein, 2644 aa, Homo sapiens
MGEHGLELASMIPALRELGSATPEEYNTVVQKPRQILCQFIDRILTDVNVVAVELVKKTDSQPTSVMLLDFIQHIMKSSPLMFVNVSGSHERKGSCIEFSNWIITRLLRIAATPSCHLLHKKICEVICSLLFLFKSKSPAIFGVLTKELLQLFEDLVYLHRRNVMGHAVEWPVVMSRFLSQLDEHMGYLQSAPLQLMSMQNLEFIEVTLLMVLTRIIAIVFFRRQELLLWQIGCVLLEYGSPKIKSLAISFLTELFQLGGLPAQPASTFFSSFLELLKHLVEMDTDQLKLYEEPLSKLIKTLFPFEAEAYRNIEPVYLNMLLEKLCVMFEDGVLMRLKSDLLKAALCHLLQYFLKFVPAGYESALQVRKVYVRNICKALLDVLGIEVDAEYLLGPLYAALKMESMEIIEEIQCQTQQENLSSNSDGISPKRRRLSSSLNPSKRAPKQTEEIKHVDMNQKSILWSALKQKAESLQISLEYSGLKNPVIEMLEGIAVVLQLTALCTVHCSHQNMNCRTFKDCQHKSKKKPSVVITWMSLDFYTKVLKSCRSLLESVQKLDLEATIDKVVKIYDALIYMQVNSSFEDHILEDLCGMLSLPWIYSHSDDGCLKLTTFAANLLTLSCRISDSYSPQAQSRCVFLLTLFPRRIFLEWRTAVYNWALQSSHEVIRASCVSGFFILLQQQNSCNRVPKILIDKVKDDSDIVKKEFASILGQLVCTLHGMFYLTSSLTEPFSEHGHVDLFCRNLKATSQHECSSSQLKASVCKPFLFLLKKKIPSPVKLAFIDNLHHLCKHLDFREDETDVKAVLGTLLNLMEDPDKDVRVAFSGNIKHILESLDSEDGFIKELFVLRMKEAYTHAQISRNNELKDTLILTTGDIGRAAKGDLVPFALLHLLHCLLSKSASVSGAAYTEIRALVAAKSVKLQSFFSQYKKPICQFLVESLHSSQMTALPNTPCQNADVRKQDVAHQREMALNTLSEIANVFDFPDLNRFLTRTLQVLLPDLAAKASPAASALIRTLGKQLNVNRREILINNFKYIFSHLVCSCSKDELERALHYLKNETEIELGSLLRQDFQGLHNELLLRIGEHYQQVFNGLSILASFASSDDPYQGPRDIISPELMADYLQPKLLGILAFFNMQLLSSSVGIEDKKMALNSLMSLMKLMGPKHVSSVRVKMMTTLRTGLRFKDDFPELCCRAWDCFVRCLDHACLGSLLSHVIVALLPLIHIQPKETAAIFHYLIIENRDAVQDFLHEIYFLPDHPELKKIKAVLQEYRKETSESTDLQTTLQLSMKAIQHENVDVRIHALTSLKETLYKNQEKLIKYATDSETVEPIISQLVTVLLKGCQDANSQARLLCGECLGELGAIDPGRLDFSTTETQGKDFTFVTGVEDSSFAYGLLMELTRAYLAYADNSRAQDSAAYAIQELLSIYDCREMETNGPGHQLWRRFPEHVREILEPHLNTRYKSSQKSTDWSGVKKPIYLSKLGSNFAEWSASWAGYLITKVRHDLASKIFTCCSIMMKHDFKVTIYLLPHILVYVLLGCNQEDQQEVYAEIMAVLKHDDQHTINTQDIASDLCQLSTQTVFSMLDHLTQWARHKFQALKAEKCPHSKSNRNKVDSMVSTVDYEDYQSVTRFLDLIPQDTLAVASFRSKAYTRAVMHFESFITEKKQNIQEHLGFLQKLYAAMHEPDGVAGVSAIRKAEPSLKEQILEHESLGLLRDATACYDRAIQLEPDQIIHYHGVVKSMLGLGQLSTVITQVNGVHANRSEWTDELNTYRVEAAWKLSQWDLVENYLAADGKSTTWSVRLGQLLLSAKKRDITAFYDSLKLVRAEQIVPLSAASFERGSYQRGYEYIVRLHMLCELEHSIKPLFQHSPGDSSQEDSLNWVARLEMTQNSYRAKEPILALRRALLSLNKRPDYNEMVGECWLQSARVARKAGHHQTAYNALLNAGESRLAELYVERAKWLWSKGDVHQALIVLQKGVELCFPENETPPEGKNMLIHGRAMLLVGRFMEETANFESNAIMKKYKDVTACLPEWEDGHFYLAKYYDKLMPMVTDNKMEKQGDLIRYIVLHFGRSLQYGNQFIYQSMPRMLTLWLDYGTKAYEWEKAGRSDRVQMRNDLGKINKVITEHTNYLAPYQFLTAFSQLISRICHSHDEVFVVLMEIIAKVFLAYPQQAMWMMTAVSKSSYPMRVNRCKEILNKAIHMKKSLEKFVGDATRLTDKLLELCNKPVDGSSSTLSMSTHFKMLKKLVEEATFSEILIPLQSVMIPTLPSILGTHANHASHEPFPGHWAYIAGFDDMVEILASLQKPKKISLKGSDGKFYIMMCKPKDDLRKDCRLMEFNSLINKCLRKDAESRRRELHIRTYAVIPLNDECGIIEWVNNTAGLRPILTKLYKEKGVYMTGKELRQCMLPKSAALSEKLKVFREFLLPRHPPIFHEWFLRTFPDPTSWYSSRSAYCRSTAVMSMVGYILGLGDRHGENILFDSLTGECVHVDFNCLFNKGETFEVPEIVPFRLTHNMVNGMGPMGTEGLFRRACEVTMRLMRDQREPLMSVLKTFLHDPLVEWSKPVKGHSKAPLNETGEVVNEKAKTHVLDIEQRLQGVIKTRNRVTGLPLSIEGHVHYLIQEATDENLLCQMYLGWTPYM

SEQ ID NO: 3 - RNA, 19 nt, Artificial Sequence
  siRNA oligonucleotide
gccaguguaugcuaccaaa

SEQ ID NO: 4 - RNA, 19 nt, Artificial Sequence
  siRNA oligonucleotide
ccgcuaaucuucuaacauu

SEQ ID NO: 5 - RNA, 19 nt, Artificial Sequence
  siRNA oligonucleotide
gcuuaagucugauuugcua

SEQ ID NO: 6 - DNA, 5205 bp, Homo sapiens
atgaagcggagccgctgccgcgaccgaccgcagccgccgccgcccgaccgccgggaggatggagttcagcgggcagcggagctgtctcagtctttgccgccgcgccggcgagcgccgcccgggaggcagcggctggaggagcggacgggccccgcggggcccgagggcaaggagcaggatgtagtaactggagttagtcccctgctcttcaggaaactcagtaatcctgacatattttcatccactggaaaagttaaacttcagcgacaactgagtcaggatgattgtaagttatggagaggaaacctggccagctctctatcgggtaagcagctgctccctttgtccagcagtgtacatagcagtgtgggacaggtgacttggcagtcgtcaggagaagcatcaaacctggttcgaatgagaaaccagtcccttggacagtctgcaccttctcttactgctggcctgaaggagttgagccttccaagaagaggcagcttttgtcggacaagtaaccgcaagagcttgattgtgacctctagcacatcacctacactaccacggccacactcaccactccatggccacacaggtaacagtcctttggacagcccccggaatttctctccaaatgcacctgctcacttttcttttgttcctgcccgtaggactgatgggcggcgctggtctttggcctctttgccctcttcaggatatggaactaacactcctagctccactgtctcatcatcatgctcctcacaggaaaagctgcatcagttgcctttccagcctacagctgatgagctgcactttttgacgaagcatttcagcacagagagcgtaccagatgaggaaggacggcagtccccagccatgcggcctcgctcccggagcctcagtcccggacgatccccagtatcctttgacagtgaaataataatgatgaatcacgtttacaaagaaagattcccaaaggccaccgcacaaatggaagagcgactagcagagtttatttcctccaacactccagacagcgtgctgcccttggcagatggagccctgagctttattcatcatcaggtgattgagatggcccgagactgcctggataaatctcggagtggcctcattacatcacaatacttctacgaacttcaagagaatttggagaaacttttacaagatgctcatgagcgctcagagagctcagaagtggcttttgtgatgcagctggtgaaaaagctgatgattatcattgcccgcccagcacgtctcctggaatgcctggagtttgaccctgaagagttctaccaccttttagaagcagctgagggccacgccaaagagggacaagggattaaatgtgacattccccgctacatcgttagccagctgggcctcacccgggatcccctagaagaaatggcccagttgagcagctgtgacagtcctgacactccagagacagatgattctattgagggccatggggcatctctgccatctaaaaagacaccctctgaagaggacttcgagaccattaagctcatcagcaatggcgcctatggggctgtatttctggtgcggcacaagtccacccggcagcgctttgccatgaagaagatcaacaagcagaacctgatcctacggaaccagatccagcaggccttcgtggagcgtgacatactgactttcgctgagaacccctttgtggtcagcatgttctgctcctttgataccaagcgccacttgtgcatggtgatggagtacgttgaagggggagactgtgccactctgctgaagaatattggggccctgcctgtggacatggtgcgtctatactttgcggaaactgtgctggccctggagtacttacacaactatggcatcgtgcaccgtgacctcaagcctgacaacctcctaattacatccatggggcacatcaagctcacggactttggactgtccaaaatgggcctcatgagtctgacaacgaacttgtatgagggtcatattgaaaaggatgcccgggaattcctggacaagcaggtatgcgggaccccagaatacattgcgcctgaggtgatcctgcgccagggctatgggaagccagtggactggtgggccatgggcattatcctgtatgagttcctggtgggctgcgtccctttttttggagatactccggaggagctctttgggcaggtgatcagtgatgagattgtgtggcctgagggtgatgaggcactgcccccagacgcccaggacctcacctccaaactgctccaccagaaccctctggagagacttggcacaggcagtgcctatgaggtgaagcagcacccattctttactggtctggactggacaggacttctccgccagaaggctgaatttattcctcagttggagtcagaggatgatactagctattttgacacccgctcagagcgataccaccacatggactcggaggatgaggaagaagtgagtgaggatggctgccttgagatccgccagttctcttcctgctctccaaggttcaacaaggtgtacagcagcatggagcggctctcactgctcgaggagcgccggacaccacccccgaccaagcgcagcctgagtgaggagaaggaggaccattcagatggcctggcagggctcaaaggccgagaccggagctgggtgattggctcccctgagatattacggaagcggctgtcggtgtctgagtcgtcccacacagagagtgactcaagccctccaatgacagtgcgacgccgctgctcaggcctcctggatgcgcctcggttcccggagggccctgaggaggccagcagcaccctcaggaggcaaccacaggagggtatatgggtcctgacacccccatctggagagggggtatctgggcctgtcactgaacactcaggggagcagcggccaaagctggatgaggaagctgttggccggagcagtggttccagtccagctatggagacccgaggccgtgggacctcacagctggctgagggagccacagccaaggccatcagtgacctggctgtgcgtagggcccgccaccggctgctctctggggactcaacagagaagcgcactgctcgccctgtcaacaaagtgatcaagtccgcctcagccacagccctctcactcctcattccttcggaacaccacacctgctccccgttggccagccccatgtccccacattctcagtcgtccaacccatcatcccgggactcttctccaagcagggacttcttgccagcccttggcagcatgaggcctcccatcatcatccaccgagctggcaagaagtatggcttcaccctgcgggccattcgcgtctacatgggtgactccgatgtctacaccgtgcaccatatggtgtggcacgtggaggatggaggtccggccagtgaggcagggcttcgtcaaggtgacctcatcacccatgtcaatggggaacctgtgcatggcctggtgcacacggaggtggtggagctgatcctgaagagtggaaacaaggtggccatttcaacaactcccctggagaacacatccattaaagtggggccagctcggaagggcagctacaaggccaagatggcccgaaggagcaagaggagccgcggcaaggatgggcaagaaagcagaaaaaggagctccctgttccgcaagatcaccaagcaagcatccctgctccacaccagccgcagcctttcttcccttaaccgctccttgtcatcaggggagagtgggccaggctctcccacacacagccacagcctttccccccgatctcccactcaaggctaccgggtgacccccgatgctgtgcattcagtgggagggaattcatcacagagcagctcccccagctccagcgtgcccagttccccagccggctctgggcacacacggcccagctccctccacggtctggcacccaagctccaacgccagtaccgctctccacggcgcaagtcagcaggcagcatcccactgtcaccactggcccacaccccttctcccccacccccaacagcttcacctcagcggtccccatcgcccctgtctggccatgtagcccaggcctttcccacaaagcttcacttgtcacctcccctgggcaggcaactctcacggcccaagagtgcggagccaccccgttcaccactactcaagagggtgcagtcggctgagaaactggcagcagcacttgccgcctctgagaagaagctagccacttctcgcaagcacagccttgacctgccccactctgaactaaagaaggaactgccgcccagggaagtgagccctctggaggtagttggagccaggagtgtgctgtctggcaagggggccctgccagggaagggggtgctgcagcctgctccctcacgggccctaggcaccctccggcaggaccgagccgaacgacgggagtcgctgcagaagcaagaagccattcgtgaggtggactcctcagaggacgacaccgaggaagggcctgagaacagccagggtgcacaggagctgagcttggcacctcacccagaagtgagccagagtgtggcccctaaaggagcaggagagagtggggaagaggatcctttcccgtccagaggccctaggagcctgggcccaatggtcccaagcctattgacagggatcacactggggcctcccagaatggaaagtcccagtggtccccacaggaggctcgggagcccacaagccattgaggaggctgccagctcctcctcagcaggccccaacctaggtcagtctggagccacagaccccatccctcctgaaggttgctggaaggcccagcacctccacacccaggcactaacagcactttctcccagcacttcgggactcacccccaccagcagttgctctcctcccagctccacctctgggaagctgagcatgtggtcctggaaatcccttattgagggcccagacagggcatccccaagcagaaaggcaaccatggcaggtgggctagccaacctccaggatttggaaacacaactccagcccagcctaagaacctgtctcccagggagcaggggaagacacagccacctagtgcccccagactggcccatccatcttatgaggatcccagccagggctggctatgggagtctgagtgtgcacaagcagtga

SEQ ID NO: 7 - protein, 1734 aa, Homo sapiens
MKRSRCRDRPQPPPPDRREDGVQRAAELSQSLPPRRRAPPGRQRLEERTGPAGPEGKEQDVVTGVSPLLFRKLSNPDIFSSTGKVKLQRQLSQDDCKLWRGNLASSLSGKQLLPLSSSVHSSVGQVTWQSSGEASNLVRMRNQSLGQSAPSLTAGLKELSLPRRGSFCRTSNRKSLIVTSSTSPTLPRPHSPLHGHTGNSPLDSPRNFSPNAPAHFSFVPARRTDGRRWSLASLPSSGYGTNTPSSTVSSSCSSQEKLHQLPFQPTADELHFLTKHFSTESVPDEEGRQSPAMRPRSRSLSPGRSPVSFDSEIIMMNHVYKERFPKATAQMEERLAEFISSNTPDSVLPLADGALSFIHHQVIEMARDCLDKSRSGLITSQYFYELQENLEKLLQDAHERSESSEVAFVMQLVKKLMIIIARPARLLECLEFDPEEFYHLLEAAEGHAKEGQGIKCDIPRYIVSQLGLTRDPLEEMAQLSSCDSPDTPETDDSIEGHGASLPSKKTPSEEDFETIKLISNGAYGAVFLVRHKSTRQRFAMKKINKQNLILRNQIQQAFVERDILTFAENPFVVSMFCSFDTKRHLCMVMEYVEGGDCATLLKNIGALPVDMVRLYFAETVLALEYLHNYGIVHRDLKPDNLLITSMGHIKLTDFGLSKMGLMSLTTNLYEGHIEKDAREFLDKQVCGTPEYIAPEVILRQGYGKPVDWWAMGIILYEFLVGCVPFFGDTPEELFGQVISDEIVWPEGDEALPPDAQDLTSKLLHQNPLERLGTGSAYEVKQHPFFTGLDWTGLLRQKAEFIPQLESEDDTSYFDTRSERYHHMDSEDEEEVSEDGCLEIRQFSSCSPRFNKVYSSMERLSLLEERRTPPPTKRSLSEEKEDHSDGLAGLKGRDRSWVIGSPEILRKRLSVSESSHTESDSSPPMTVRRRCSGLLDAPRFPEGPEEASSTLRRQPQEGIWVLTPPSGEGVSGPVTEHSGEQRPKLDEEAVGRSSGSSPAMETRGRGTSQLAEGATAKAISDLAVRRARHRLLSGDSTEKRTARPVNKVIKSASATALSLLIPSEHHTCSPLASPMSPHSQSSNPSSRDSSPSRDFLPALGSMRPPIIIHRAGKKYGFTLRAIRVYMGDSDVYTVHHMVWHVEDGGPASEAGLRQGDLITHVNGEPVHGLVHTEVVELILKSGNKVAISTTPLENTSIKVGPARKGSYKAKMARRSKRSRGKDGQESRKRSSLFRKITKQASLLHTSRSLSSLNRSLSSGESGPGSPTHSHSLSPRSPTQGYRVTPDAVHSVGGNSSQSSSPSSSVPSSPAGSGHTRPSSLHGLAPKLQRQYRSPRRKSAGSIPLSPLAHTPSPPPPTASPQRSPSPLSGHVAQAFPTKLHLSPPLGRQLSRPKSAEPPRSPLLKRVQSAEKLAAALAASEKKLATSRKHSLDLPHSELKKELPPREVSPLEVVGARSVLSGKGALPGKGVLQPAPSRALGTLRQDRAERRESLQKQEAIREVDSSEDDTEEGPENSQGAQELSLAPHPEVSQSVAPKGAGESGEEDPFPSRGPRSLGPMVPSLLTGITLGPPRMESPSGPHRRLGSPQAIEEAASSSSAGPNLGQSGATDPIPPEGCWKAQHLHTQALTALSPSTSGLTPTSSCSPPSSTSGKLSMWSWKSLIEGPDRASPSRKATMAGGLANLQDLETQLQPSLRTCLPGSRGRHSHLVPPDWPIHLMRIPARAGYGSLSVHKQ

SEQ ID NO: 8 - RNA, 19 nt, Artificial Sequence
  siRNA oligonucleotide
guaugagggucauauugaa

SEQ ID NO: 9 - RNA, 19 nt, Artificial Sequence
  siRNA oligonucleotide
ccgccagaaggcugaauuu

SEQ ID NO: 10 - RNA, 19 nt, Artificial Sequence
  siRNA oligonucleotide
cagucugcaccuucucuua

SEQ ID NO: 11 - DNA, 3867 bp, Homo sapiens
atggcggggccgtgtccccggtccggggcggagcgcgccggcagctgctggcaggacccgctggccgtggcgctgagccggggccggcagctcgcggcgcccccgggccggggctgcgcgcggagccggccgctcagcgtggtctacgtgctgacccgggagccgcagcccgggctcgagcctcgggagggaaccgaggcggagccgctgcccctgcgctgcctgcgcgaggcttgcgcgcaggtcccccggccgcggccgcccccgcagctgcgcagcctgcccttcgggacgctggagctaggcgacaccgcggctctggatgccttctacaacgcggatgtggtggtgctggaggtgagcagctcgctggtacagccctccctgttctaccaccttggtgtgcgtgagagcttcagcatgaccaacaatgtgctcctctgctcccaggccgacctccctgacctgcaggccctgcgggaggatgttttccagaagaactcggattgcgttggcagctacacactgatcccctatgtggtgacggccactggtcgggtgctgtgtggtgatgcaggccttctgcggggcctggctgatgggctggtacaggctggagtggggaccgaggccctgctcactcccctggtgggccggcttgcccgcctgctggaggccacacccacagactcttgtggctatttccgggagaccattcggcgggacatccggcaggcgcgggagcggttcagtgggccacagctgcggcaggagctggctcgcctgcagcggagactggacagcgtggagctgctgagccccgacatcatcatgaacttgctgctctcctaccgcgatgtgcaggactactcggccatcattgagctggtggagacgctgcaggccttgcccacctgtgatgtggccgagcagcataatgtctgcttccactacacttttgccctcaaccggaggaacaggcctggggaccgggcgaaggccctgtctgtgctgctgccgctggtacagcttgagggctctgtggcgcccgatctgtactgcatgtgtggccgtatctacaaggacatgttcttcagctcgggtttccaggatgctgggcaccgggagcaggcctatcactggtatcgcaaggcttttgacgtagagcccagccttcactcaggcatcaatgcagctgtgctcctcattgctgccgggcagcactttgaggattccaaagagctccggctaataggcatgaagctgggctgcctgctggcccgcaaaggctgcgtggagaagatgcagtattactgggatgtgggtttctacctgggagcccagatcctcgccaatgaccccacccaggtggtgctggctgcagagcagctgtataagctcaatgcccccatatggtacctggtgtccgtgatggagaccttcctgctctaccagcacttcaggcccacgccagagccccctggagggccaccacgccgtgcccacttctggctccacttcttgctacagtcctgccaaccattcaagacagcctgtgcccagggcgaccagtgcttggtgctggtcctggagatgaacaaggtgctgctgcctgcaaagctcgaggttcggggtactgacccagtaagcacagtgaccctgagcctgctggagcctgagacccaggacattccctccagctggaccttcccagtcgcctccatatgcggagtcagcgcctcaaagcgcgacgagcgctgctgcttcctctatgcactccccccggctcaggacgtccagctgtgcttccccagcgtagggcactgccagtggttctgcggcctgatccaggcctgggtgacgaacccggattccacggcgcccgcggaggaggcggagggcgcgggggagatgttggagtttgattatgagtacacggagacgggcgagcggctggtgctgggcaagggcacgtatggggtggtgtacgcgggccgcgatcgccacacgagggtgcgcatcgccatcaaggagatcccggagcgggacagcaggttctctcagcccctgcatgaagagatcgctcttcacagacgcctgcgccacaagaacatagtgcgctatctgggctcagctagccagggcggctaccttaagatcttcatggaggaagtgcctggaggcagcctgtcctccttgctgcggtcggtgtggggacccctgaaggacaacgagagcaccatcagtttctacacccgccagatcctgcagggacttggctacttgcacgacaaccacatcgtgcacagggacataaaaggggacaatgtgctgatcaacaccttcagtgggctgctcaagatttctgacttcggcacctccaagcggctggcaggcatcacaccttgcactgagaccttcacaggaactctgcagtatatggccccagaaatcattgaccagggcccacgcgggtatgggaaagcagctgacatctggtcactgggctgcactgtcattgagatggccacaggtcgcccccccttccacgagctcgggagcccacaggctgccatgtttcaggtgggtatgtacaaggtccatccgccaatgcccagctctctgtcggccgaggcccaagcctttctcctccgaacttttgagccagacccccgcctccgagccagcgcccagacactgctgggggaccccttcctgcagcctgggaaaaggagccgcagccccagctccccacgacatgctccacggccctcagatgccccttctgccagtcccactccttcagccaactcaaccacccagtctcagacattcccgtgccctcaggcaccctctcagcacccacccagccccccgaagcgctgcctcagttatgggggcaccagccagctccgggtgcccgaggagcctgcggccgaggagcctgcgtctccggaggagagttcggggctgagcctgctgcaccaggagagcaagcgtcgggccatgctggccgcagtattggagcaggagctgccagcgctggcggagaatctgcaccaggagcagaagcaagagcagggggcccgtctgggcagaaaccatgtggaagagctgctgcgctgcctcggggcacacatccacactcccaaccgccggcagctcgcccaggagctgcgggcgctgcaaggacggctgagggcccagggccttgggcctgcgcttctgcacagaccgctgtttgccttcccggatgcggtgaagcagatcctccgcaagcgccagatccgtccacactggatgttcgttctggactcactgctcagccgtgctgtgcgggcagccctgggtgtgctaggaccggaggtggagaaggaggcggtctcaccgaggtcagaggagctgagtaatgaaggggactcccagcagagcccaggccagcagagcccgcttccggtggagcccgagcagggccccgctcctctgatggtgcagctgagcctcttgagggcagagactgatcggctgcgcgaaatcctggcggggaaggaacgggagtaccaggccctggtgcagcgggctctacagcggctgaatgaggaagcccggacctatgtcctggccccagagcctccaactgctctttcaacggaccagggcctggtgcagtggctacaggaactgaatgtggattcaggcaccatccaaatgctgttgaaccatagcttcaccctccacactctgctcacctatgccactcgagatgacctcatctacacccgcatcaggggagggatggtatgccgcatctggagggccatcttggcacagcgagcaggatccacaccagtcacctctggaccctga

SEQ ID NO: 12 - protein, 1288 aa, Homo sapiens
MAGPCPRSGAERAGSCWQDPLAVALSRGRQLAAPPGRGCARSRPLSVVYVLTREPQPGLEPREGTEAEPLPLRCLREACAQVPRPRPPPQLRSLPFGTLELGDTAALDAFYNADVVVLEVSSSLVQPSLFYHLGVRESFSMTNNVLLCSQADLPDLQALREDVFQKNSDCVGSYTLIPYVVTATGRVLCGDAGLLRGLADGLVQAGVGTEALLTPLVGRLARLLEATPTDSCGYFRETIRRDIRQARERFSGPQLRQELARLQRRLDSVELLSPDIIMNLLLSYRDVQDYSAIIELVETLQALPTCDVAEQHNVCFHYTFALNRRNRPGDRAKALSVLLPLVQLEGSVAPDLYCMCGRIYKDMFFSSGFQDAGHREQAYHWYRKAFDVEPSLHSGINAAVLLIAAGQHFEDSKELRLIGMKLGCLLARKGCVEKMQYYWDVGFYLGAQILANDPTQVVLAAEQLYKLNAPIWYLVSVMETFLLYQHFRPTPEPPGGPPRRAHFWLHFLLQSCQPFKTACAQGDQCLVLVLEMNKVLLPAKLEVRGTDPVSTVTLSLLEPETQDIPSSWTFPVASICGVSASKRDERCCFLYALPPAQDVQLCFPSVGHCQWFCGLIQAWVTNPDSTAPAEEAEGAGEMLEFDYEYTETGERLVLGKGTYGVVYAGRDRHTRVRIAIKEIPERDSRFSQPLHEEIALHRRLRHKNIVRYLGSASQGGYLKIFMEEVPGGSLSSLLRSVWGPLKDNESTISFYTRQILQGLGYLHDNHIVHRDIKGDNVLINTFSGLLKISDFGTSKRLAGITPCTETFTGTLQYMAPEIIDQGPRGYGKAADIWSLGCTVIEMATGRPPFHELGSPQAAMFQVGMYKVHPPMPSSLSAEAQAFLLRTFEPDPRLRASAQTLLGDPFLQPGKRSRSPSSPRHAPRPSDAPSASPTPSANSTTQSQTFPCPQAPSQHPPSPPKRCLSYGGTSQLRVPEEPAAEEPASPEESSGLSLLHQESKRRAMLAAVLEQELPALAENLHQEQKQEQGARLGRNHVEELLRCLGAHIHTPNRRQLAQELRALQGRLRAQGLGPALLHRPLFAFPDAVKQILRKRQIRPHWMFVLDSLLSRAVRAALGVLGPEVEKEAVSPRSEELSNEGDSQQSPGQQSPLPVEPEQGPAPLMVQLSLLRAETDRLREILAGKEREYQALVQRALQRLNEEARTYVLAPEPPTALSTDQGLVQWLQELNVDSGTIQMLLNHSFTLHTLLTYATRDDLIYTRIRGGMVCRIWRAILAQRAGSTPVTSGP

SEQ ID NO: 13 - RNA, 19 nt, Artificial Sequence
  siRNA oligonucleotide
gcacuuugaggauuccaaa

SEQ ID NO: 14 - RNA, 19 nt, Artificial Sequence
  siRNA oligonucleotide
cgcccgaucuguacugcau

SEQ ID NO: 15 - RNA, 19 nt, Artificial Sequence
  siRNA oligonucleotide
gagauguuggaguuugauu

SEQ ID NO: 16 - DNA, 2190 bp, Homo sapiens
atgcagagcacttctaatcatctgtggcttttatctgatattttaggccaaggagctactgcaaatgtctttcgtggaagacataagaaaactggtgatttatttgctatcaaagtatttaataacataagcttccttcgtccagtggatgttcaaatgagagaatttgaagtgttgaaaaaactcaatcacaaaaatattgtcaaattatttgctattgaagaggagacaacaacaagacataaagtacttattatggaattttgtccatgtgggagtttatacactgttttagaagaaccttctaatgcctatggactaccagaatctgaattcttaattgttttgcgagatgtggtgggtggaatgaatcatctacgagagaatggtatagtgcaccgtgatatcaagccaggaaatatcatgcgtgttataggggaagatggacagtctgtgtacaaactcacagattttggtgcagctagagaattagaagatgatgagcagtttgtttctctgtatggcacagaagaatatttgcaccctgatatgtatgagagagcagtgctaagaaaagatcatcagaagaaatatggagcaacagttgatctttggagcattggggtaacattttaccatgcagctactggatcactgccatttagaccctttgaagggcctcgtaggaataaagaagtgatgtataaaataattacaggaaagccttctggtgcaatatctggagtacagaaagcagaaaatggaccaattgactggagtggagacatgcctgtttcttgcagtctttctcggggtcttcaggttctacttacccctgttcttgcaaacatccttgaagcagatcaggaaaagtgttggggttttgaccagttttttgcagaaactagtgatatacttcaccgaatggtaattcatgttttttcgctacaacaaatgacagctcataagatttatattcatagctataatactgctactatatttcatgaactggtatataaacaaaccaaaattatttcttcaaatcaagaacttatctacgaagggcgacgcttagtcttagaacctggaaggctggcacaacatttccctaaaactactgaggaaaaccctatatttgtagtaagccgggaacctctgaataccataggattaatatatgaaaaaatttccctccctaaagtacatccacgttatgatttagacggggatgctagcatggctaaggcaataacaggggttgtgtgttatgcctgcagaattgccagtaccttactgctttatcaggaattaatgcgaaaggggatacgatggctgattgaattaattaaagatgattacaatgaaactgttcacaaaaagacagaagttgtgatcacattggatttctgtatcagaaacattgaaaaaactgtgaaagtatatgaaaagttgatgaagatcaacctggaagcggcagagttaggtgaaatttcagacatacacaccaaattgttgagactttccagttctcagggaacaatagaaaccagtcttcaggatatcgacagcagattatctccaggtggatcactggcagacgcatgggcacatcaagaaggcactcatccgaaagacagaaatgtagaaaaactacaagtcctgttaaattgcatgacagagatttactatcagttcaaaaaagacaaagcagaacgtagattagcttataatgaagaacaaatccacaaatttgataagcaaaaactgtattaccatgccacaaaagctatgacgcactttacagatgaatgtgttaaaaagtatgaggcatttttgaataagtcagaagaatggataagaaagatgcttcatcttaggaaacagttattatcgctgactaatcagtgttttgatattgaagaagaagtatcaaaatatcaagaatatactaatgagttacaagaaactctgcctcagaaaatgtttacagcttccagtggaatcaaacataccatgaccccaatttatccaagttctaacacattagtagaaatgactcttggtatgaagaaattaaaggaagagatggaaggggtggttaaagaacttgctgaaaataaccacattttagaaaggtttggctctttaaccatggatggtggccttcgcaacgttgactgtctttag

SEQ ID NO: 17 - protein, 729 aa, Homo sapiens
MQSTSNHLWLLSDILGQGATANVFRGRHKKTGDLFAIKVFNNISFLRPVDVQMREFEVLKKLNHKNIVKLFAIEEETTTRHKVLIMEFCPCGSLYTVLEEPSNAYGLPESEFLIVLRDVVGGMNHLRENGIVHRDIKPGNIMRVIGEDGQSVYKLTDFGAARELEDDEQFVSLYGTEEYLHPDMYERAVLRKDHQKKYGATVDLWSIGVTFYHAATGSLPFRPFEGPRRNKEVMYKIITGKPSGAISGVQKAENGPIDWSGDMPVSCSLSRGLQVLLTPVLANILEADQEKCWGFDQFFAETSDILHRMVIHVFSLQQMTAHKIYIHSYNTATIFHELVYKQTKIISSNQELIYEGRRLVLEPGRLAQHFPKTTEENPIFVVSREPLNTIGLIYEKISLPKVHPRYDLDGDASMAKAITGVVCYACRIASTLLLYQELMRKGIRWLIELIKDDYNETVHKKTEVVITLDFCIRNIEKTVKVYEKLMKINLEAAELGEISDIHTKLLRLSSSQGTIETSLQDIDSRLSPGGSLADAWAHQEGTHPKDRNVEKLQVLLNCMTEIYYQFKKDKAERRLAYNEEQIHKFDKQKLYYHATKAMTHFTDECVKKYEAFLNKSEEWIRKMLHLRKQLLSLTNQCFDIEEEVSKYQEYTNELQETLPQKMFTASSGIKHTMTPIYPSSNTLVEMTLGMKKLKEEMEGVVKELAENNHILERFGSLTMDGGLRNVDCL

SEQ ID NO: 18 - RNA, 19 nt, Artificial Sequence
  siRNA oligonucleotide
gcggcagaguuaggugaaa

SEQ ID NO: 19 - RNA, 19 nt, Artificial Sequence
  siRNA oligonucleotide
ccacaaauuugauaagcaa

SEQ ID NO: 20 - RNA, 19 nt, Artificial Sequence
  siRNA oligonucleotide
cucugaauaccauaggauu

SEQ ID NO: 21 - DNA, 2067 bp, Homo sapiens
atggcggacctggaggccgtgctggccgatgtcagttacctgatggccatggagaagagcaaggcgaccccggccgcccgcgccagcaagaggatcgtcctgccggagcccagtatccggagtgtgatgcagaagtaccttgcagagagaaatgaaataacctttgacaagattttcaatcagaaaattggtttcttgctatttaaagatttttgtttgaatgaaattaatgaagctgtacctcaggtgaagttttatgaagagataaaggaatatgaaaaacttgataatgaggaagaccgcctttgcagaagtcgacaaatttatgatgcctacatcatgaaggaacttctttcctgttcacatcctttctcaaagcaagctgtagaacacgtacaaagtcatttatccaagaaacaagtgacatcaactctttttcagccatacatagaagaaatttgtgaaagccttcgaggtgacatttttcaaaaatttatggaaagtgacaagttcactagattttgtcagtggaaaaacgttgaattaaatatccatttgaccatgaatgagttcagtgtgcataggattattggacgaggaggattcggggaagtttatggttgcaggaaagcagacactggaaaaatgtatgcaatgaaatgcttagataagaagaggatcaaaatgaaacaaggagaaacattagctttaaatgaaagaatcatgttgtctcttgtcagcacaggagactgtcctttcattgtatgtatgacctatgccttccataccccagataaactctgcttcatcctggatctgatgaacgggggcgatttgcactaccacctttcacaacacggtgtgttctctgagaaggagatgcggttttatgccactgaaatcattctgggtctggaacacgtgcacaatcggtttgttgtctacagagatttgaagccagcaaatattctcttggatgaacatggacacgcaagaatatcagatcttggtcttgcctgcgatttttccaaaaagaagcctcatgcgagtgttggcacccatgggtacatggctcccgaggtgctgcagaaggggacggcctatgacagcagtgccgactggttctccctgggctgcatgcttttcaaacttctgagaggtcacagccctttcagacaacataaaaccaaagacaagcatgaaattgaccgaatgacactcaccgtgaatgtggaacttccagacaccttctctcctgaactgaagtcccttttggagggcttgcttcagcgagacgttagcaagcggctgggctgtcacggaggcggctcacaggaagtaaaagagcacagctttttcaaaggtgttgactggcagcatgtctacttacaaaagtacccaccacccttgattcctccccggggagaagtcaatgctgctgatgcctttgatattggctcatttgatgaagaggataccaaagggattaagctacttgattgcgaccaagaactctacaagaacttccctttggtcatctctgaacgctggcagcaagaagtaacggaaacagtttatgaagcagtaaatgcagacacagataaaatcgaggccaggaagagagctaaaaataagcaacttggccacgaagaagattacgctctggggaaggactgtattatgcacgggtacatgctgaaactgggaaacccatttctgactcagtggcagcgtcgctatttttacctctttccaaatagacttgaatggagaggagagggagagtcccggcaaaatttactgacaatggaacagattctctctgtggaagaaactcaaattaaagacaaaaaatgcattttgttcagaataaaaggagggaaacaatttgtcttgcaatgtgagagtgatccagagtttgtgcagtggaagaaagagttgaacgaaaccttcaaggaggcccagcggctattgcgtcgtgccccgaagttcctcaacaaacctcggtcaggtactgtggagctcccaaagccatccctctgtcacaggaacagcaacggcctctga

SEQ ID NO: 22 - protein, 688 aa, Homo sapiens
MADLEAVLADVSYLMAMEKSKATPAARASKRIVLPEPSIRSVMQKYLAERNEITFDKIFNQKIGFLLFKDFCLNEINEAVPQVKFYEEIKEYEKLDNEEDRLCRSRQIYDAYIMKELLSCSHPFSKQAVEHVQSHLSKKQVTSTLFQPYIEEICESLRGDIFQKFMESDKFTRFCQWKNVELNIHLTMNEFSVHRIIGRGGFGEVYGCRKADTGKMYAMKCLDKKRIKMKQGETLALNERIMLSLVSTGDCPFIVCMTYAFHTPDKLCFILDLMNGGDLHYHLSQHGVFSEKEMRFYATEIILGLEHVHNRFVVYRDLKPANILLDEHGHARISDLGLACDFSKKKPHASVGTHGYMAPEVLQKGTAYDSSADWFSLGCMLFKLLRGHSPFRQHKTKDKHEIDRMTLTVNVELPDTFSPELKSLLEGLLQRDVSKRLGCHGGGSQEVKEHSFFKGVDWQHVYLQKYPPPLIPPRGEVNAADAFDIGSFDEEDTKGIKLLDCDQELYKNFPLVISERWQQEVTETVYEAVNADTDKIEARKRAKNKQLGHEEDYALGKDCIMHGYMLKLGNPFLTQWQRRYFYLFPNRLEWRGEGESRQNLLTMEQILSVEETQIKDKKCILFRIKGGKQFVLQCESDPEFVQWKKELNETFKEAQRLLRRAPKFLNKPRSGTVELPKPSLCHRNSNGL

SEQ ID NO: 23 - RNA, 19 nt, Artificial Sequence
  siRNA oligonucleotide
gcaaugaaaugcuuagaua

SEQ ID NO: 24 - RNA, 19 nt, Artificial Sequence
  siRNA oligonucleotide
guccuuucauuguauguau

SEQ ID NO: 25 - RNA, 19 nt, Artificial Sequence
  siRNA oligonucleotide
gcugcugaugccuuugaua

SEQ ID NO: 26 - DNA, 1482 bp, Homo sapiens
atggaaaaatatgaaaacctgggtttggttggagaagggagttatggaatggtgatgaagtgtaggaataaagatactggaagaattgtggccataaagaagttcttagaaagtgacgatgacaaaatggttaaaaagattgcaatgcgagaaatcaagttactaaagcaacttaggcatgaaaacttggtgaatctcttggaagtgtgtaagaaaaaaaaacgatggtacctagtctttgaatttgttgaccacacaattcttgatgacttggagctctttccaaatggactagactaccaagtagttcaaaagtatttgtttcagattattaatggaattggattttgtcacagtcacaatatcatacacagagatataaagccagagaatatattagtctcccagtctggcgttgtcaagctatgcgattttggatttgcgcgaacattggcagctcctggggaggtttatactgattatgtggcaacccgatggtacagagctccagaactattggttggtgatgtcaagtatggcaaggctgttgatgtgtgggccattggttgtctggtaactgaaatgttcatgggggaacccctatttcctggagattctgatattgatcagctatatcatattatgatgtgtttaggtaatctaattccaaggcatcaggagctttttaataaaaatcctgtgtttgctggagtaaggttgcctgaaatcaaggaaagagaacctcttgaaagacgctatcctaagctctctgaagtggtgatagatttagcaaagaaatgcttacatattgaccccgacaaaagacccttctgtgctgagctcctacaccatgatttctttcaaatggatggatttgctgagaggttttcccaagaactacagttaaaagtacagaaagatgccagaaatgtttctttatctaaaaaatcccaaaacagaaagaaggaaaaagaaaaagatgattccttagttgaagaaagaaaaacacttgtggtacaggataccaatgctgatcccaaaattaaggattataaactatttaaaataaaaggctcaaaaattgatggagaaaaagctgaaaaaggcaatagagcttcaaatgccagctgtctccatgacagtaggacaagccacaacaaaatagtgccttcaacaagcctcaaagactgcagcaatgtcagcgtggaccacacaaggaatccaagcgtggcaattcccccacttacacacaatctttctgcagttgctcccagcattaattctggaatggggactgagactataccaattcagggttacagagtggatgagaaaactaagaagtgttctattccatttgttaaaccgaacagacattccccatcaggcatttataacattaatgtgaccacattagtatcaggacctcccctgtcagatgattcaggggctgatttgcctcaaatggaacaccagcactga

SEQ ID NO: 27 - protein, 493 aa, Homo sapiens
MEKYENLGLVGEGSYGMVMKCRNKDTGRIVAIKKFLESDDDKMVKKIAMREIKLLKQLRHENLVNLLEVCKKKKRWYLVFEFVDHTILDDLELFPNGLDYQVVQKYLFQIINGIGFCHSHNIIHRDIKPENILVSQSGVVKLCDFGFARTLAAPGEVYTDYVATRWYRAPELLVGDVKYGKAVDVWAIGCLVTEMFMGEPLFPGDSDIDQLYHIMMCLGNLIPRHQELFNKNPVFAGVRLPEIKEREPLERRYPKLSEVVIDLAKKCLHIDPDKRPFCAELLHHDFFQMDGFAERFSQELQLKVQKDARNVSLSKKSQNRKKEKEKDDSLVEERKTLVVQDTNADPKIKDYKLFKIKGSKIDGEKAEKGNRASNASCLHDSRTSHNKIVPSTSLKDCSNVSVDHTRNPSVAIPPLTHNLSAVAPSINSGMGTETIPIQGYRVDEKTKKCSIPFVKPNRHSPSGIYNINVTTLVSGPPLSDDSGADLPQMEHQH

SEQ ID NO: 28 - RNA, 19 nt, Artificial Sequence
  siRNA oligonucleotide
gugaugaaguguaggaaua

SEQ ID NO: 29 - RNA, 19 nt, Artificial Sequence
  siRNA oligonucleotide
gccauugguugucugguaa

SEQ ID NO: 30 - RNA, 19 nt, Artificial Sequence
  siRNA oligonucleotide
guuacagaguggaugagaa

SEQ ID NO: 31 - DNA, 3267 bp, Homo sapiens
atgaggccaaagacttttcctgccacgacttattctggaaatagccggcagcgactgcaagagattcgtgaggggttaaaacagccatccaagtcttcggttcaggggctacccgcaggaccaaacagtgacacttccctggatgccaaagtcctggggagcaaagatgccaccaggcagcagcagcagatgagagccaccccaaagttcggaccttatcagaaagccttgagggaaatcagatattccttgttgccttttgctaatgaatcgggcacctctgcagctgcagaagtgaaccggcaaatgctgcaggaactggtgaacgcaggatgcgaccaggagatggctggccgagctctcaagcagactggcagcaggagcatcgaggccgccctggagtacatcagcaagatgggctacctggacccgaggaatgagcagattgtgcgggtcattaagcagacctccccaggaaaggggctcatgccaaccccagtgacgcggaggcccagcttcgaaggaaccggcgattcgtttgcgtcctaccaccagctgagcggtaccccctacgagggcccaagcttcggcgctgacggccccacggcgctggaggagatgccgcggccgtacgtggactaccttttccccggagtcggcccccacgggcccggccaccagcaccagcacccacccaagggctacggtgccagcgtagaggcagcaggggcacacttcccgctgcagggcgcgcactacgggcggccgcacctgctggtgcctggggaacccctgggctacggagtgcagcgcagcccctccttccagagcaagacgccgccggagaccgggggttacgccagcctgcccacgaagggccagggaggaccgccaggcgccggcctcgctttcccaccccctgccgccgggctctacgtgccgcacccacaccacaagcaggccggtcccgcggcccaccagctgcatgtgctgggctcccgcagccaggtgttcgccagcgacagccccccgcagagcctgctcactccctcgcggaacagcctcaacgtggacctgtatgaattgagcagcacctccgtccagcagtggccggctgccaccctggcccgccgggactccctgcagaagccgggcctggaggcgccgccgcgcgcgcacgtggccttccggcctgactgcccagtgcccagcaggaccaactccttcaacagccaccagccgcggcccggtccgcctggcaaggccgagccctccctgcccgcccccaacaccgtgacggctgtcacggccgcgcacatcttgcacccggtgaagagcgtgcgtgtgctgaggccggagccgcagacggctgtggggccctcgcaccccgcctgggtgcccgcgcctgccccggcccccgcccccgcccccgccccggctgcggagggcttggacgccaaggaggagcatgccctggcgctgggcggcgcaggcgccttcccgctggacgtggagtacggaggcccagaccggaggtgcccgcctccgccctacccgaagcacctgctgctgcgcagcaagtcggagcagtacgacctggacagcctgtgcgcaggcatggagcagagcctccgtgcgggccccaacgagcccgagggcggcgacaagagccgcaaaagcgccaagggggacaaaggcggaaaggataaaaagcagattcagacctctcccgttcccgtccgcaaaaacagcagagacgaagagaagagagagtcacgcatcaagagctactcgccatacgcctttaagttcttcatggagcagcacgtggagaatgtcatcaaaacctaccagcagaaggttaaccggaggctgcagctggagcaagaaatggccaaagctggactctgtgaagctgagcaggagcagatgcggaagatcctctaccagaaagagtctaattacaacaggttaaagagggccaagatggacaagtctatgtttgtcaagatcaaaaccctggggatcggtgcctttggagaagtgtgccttgcttgtaaggtggacactcacgccctgtacgccatgaagaccctaaggaaaaaggatgtcctgaaccggaatcaggtggtccacgtcaaggccgagagggacatcctggccgaggcagacaatgagtgggtggtcaaactctactactccttccaagacaaagacagcctgtactttgtgatggactacatccctggtggggacatgatgagcctgctgatccggatggaggtcttccctgagcacctggcccggttctacatcgcagagctgactttggccattgagagtgtccacaagatgggcttcatccaccgagacatcaagcctgataacattttgatagatctggatggtcacattaaactcacagatttcggcctctgcactgggttcaggtggactcacaattccaaatattaccagaaagggagccatgtcagacaggacagcatggagcccagcgacctctgggatgatgtgtctaactgtcggtgtggggacaggctgaagaccctagagcagagggcgcggaagcagcaccagaggtgcctggcacattcactggtggggactccaaactacatcgcacccgaggtgctcctccgcaaagggtacactcaactctgtgactggtggagtgttggagtgattctcttcgagatgctggtggggcagccgccctttttggcacctactcccacagaaacccagctgaaggtgatcaactgggagaacacgctccacattccagcccaggtgaagctgagccctgaggccagggacctcatcaccaagctgtgctgctccgcagaccaccgcctggggcggaatggggccgatgacctgaaggcccaccccttcttcagcgccattgacttctccagtgacatccggaagcagccagccccctacgttcccaccatcagccaccccatggacacctcgaatttcgaccccgtagatgaagaaagcccttggaacgatgccagcgaaggtagcaccaaggcctgggacacactcacctcgcccaataacaagcatcctgagcacgcattttacgaattcaccttccgaaggttctttgatgacaatggctacccctttcgatgcccaaagccttcaggagcagaagcttcacaggctgagagctcagatttagaaagctctgatctggtggatcagactgaaggctgccagcctgtgtacgtgtag

SEQ ID NO: 32 - protein, 1088 aa, Homo sapiens
MRPKTFPATTYSGNSRQRLQEIREGLKQPSKSSVQGLPAGPNSDTSLDAKVLGSKDATRQQQQMRATPKFGPYQKALREIRYSLLPFANESGTSAAAEVNRQMLQELVNAGCDQEMAGRALKQTGSRSIEAALEYISKMGYLDPRNEQIVRVIKQTSPGKGLMPTPVTRRPSFEGTGDSFASYHQLSGTPYEGPSFGADGPTALEEMPRPYVDYLFPGVGPHGPGHQHQHPPKGYGASVEAAGAHFPLQGAHYGRPHLLVPGEPLGYGVQRSPSFQSKTPPETGGYASLPTKGQGGPPGAGLAFPPPAAGLYVPHPHHKQAGPAAHQLHVLGSRSQVFASDSPPQSLLTPSRNSLNVDLYELSSTSVQQWPAATLARRDSLQKPGLEAPPRAHVAFRPDCPVPSRTNSFNSHQPRPGPPGKAEPSLPAPNTVTAVTAAHILHPVKSVRVLRPEPQTAVGPSHPAWVPAPAPAPAPAPAPAAEGLDAKEEHALALGGAGAFPLDVEYGGPDRRCPPPPYPKHLLLRSKSEQYDLDSLCAGMEQSLRAGPNEPEGGDKSRKSAKGDKGGKDKKQIQTSPVPVRKNSRDEEKRESRIKSYSPYAFKFFMEQHVENVIKTYQQKVNRRLQLEQEMAKAGLCEAEQEQMRKILYQKESNYNRLKRAKMDKSMFVKIKTLGIGAFGEVCLACKVDTHALYAMKTLRKKDVLNRNQVVHVKAERDILAEADNEWVVKLYYSFQDKDSLYFVMDYIPGGDMMSLLIRMEVFPEHLARFYIAELTLAIESVHKMGFIHRDIKPDNILIDLDGHIKLTDFGLCTGFRWTHNSKYYQKGSHVRQDSMEPSDLWDDVSNCRCGDRLKTLEQRARKQHQRCLAHSLVGTPNYIAPEVLLRKGYTQLCDWWSVGVILFEMLVGQPPFLAPTPTETQLKVINWENTLHIPAQVKLSPEARDLITKLCCSADHRLGRNGADDLKAHPFFSAIDFSSDIRKQPAPYVPTISHPMDTSNFDPVDEESPWNDASEGSTKAWDTLTSPNNKHPEHAFYEFTFRRFFDDNGYPFRCPKPSGAEASQAESSDLESSDLVDQTEGCQPVYV

SEQ ID NO: 33 - RNA, 19 nt, Artificial Sequence
  siRNA oligonucleotide
ccaugaagacccuaaggaa

SEQ ID NO: 34 - RNA, 19 nt, Artificial Sequence
  siRNA oligonucleotide
gaccuuaucagaaagccuu

SEQ ID NO: 35 - RNA, 19 nt, Artificial Sequence
  siRNA oligonucleotide
gccucaacguggaccugua

SEQ ID NO: 36 - DNA, 1245 bp, Homo sapiens
atgggcgggaaccactcccacaagccccccgtgtttgacgagaatgaggaagtcaactttgaccattttcagattctgcgggccattggtaaagggagttttggaaaggtatgcatcgtgcagaagcgagacactaagaaaatgtatgcaatgaagtacatgaacaagcagaagtgcatcgagagggatgaggttcggaatgttttccgggagctgcagatcatgcaagggctggagcaccccttcctggtcaatctgtggtactccttccaggatgaggaggacatgttcatggtggtggacctgctcctgggaggcgacctgcgctaccatctgcagcagaatgtgcatttcacagaggggactgtgaaactctacatctgtgagctggcactggccctggagtatcttcagaggtaccacatcatccacagagacatcaagccagacaatatcctgctggatgaacacggacatgttcacattacagacttcaacatagcgacggtagtgaaaggagcagaaagggcttcctccatggctggcaccaagccctacatggctccagaagtattccaggtgtacatggacagaggccccggatactcgtaccctgtcgactggtggtccctgggcatcacagcctatgagctgctgcggggctggaggccgtacgaaatccactcggtcacgcccatcgatgaaatcctcaacatgttcaaggtggagcgtgtccactactcctccacgtggtgcaaggggatggtggccctgctgaggaagctcctgaccaaggatcctgagagccgcgtgtccagccttcatgacatacagagcgtgccctacttggccgacatgaactgggacgcggtgttcaagaaggcactgatgcccggctttgtgcccaataaagggaggttgaactgcgatcccacatttgagcttgaagagatgattctagaatccaagccacttcacaaaaagaagaagcgattggcaaagaacagatccagggatggcacaaaggacagctgcccgctgaatggacacctgcagcactgtttggagactgtccgggaggaattcatcatattcaacagagagaagctcaggaggcagcagggacagggcagccagctcttggacaccgacagccgagggggaggccaggcccaaagcaagctccaggacgggtgcaacaacaacctcctcacccacacctgcacccgtggctgcagcagctga

SEQ ID NO: 37 - protein, 414 aa, Homo sapiens
MGGNHSHKPPVFDENEEVNFDHFQILRAIGKGSFGKVCIVQKRDTKKMYAMKYMNKQKCIERDEVRNVFRELQIMQGLEHPFLVNLWYSFQDEEDMFMVVDLLLGGDLRYHLQQNVHFTEGTVKLYICELALALEYLQRYHIIHRDIKPDNILLDEHGHVHITDFNIATVVKGAERASSMAGTKPYMAPEVFQVYMDRGPGYSYPVDWWSLGITAYELLRGWRPYEIHSVTPIDEILNMFKVERVHYSSTWCKGMVALLRKLLTKDPESRVSSLHDIQSVPYLADMNWDAVFKKALMPGFVPNKGRLNCDPTFELEEMILESKPLHKKKKRLAKNRSRDGTKDSCPLNGHLQHCLETVREEFIIFNREKLRRQQGQGSQLLDTDSRGGGQAQSKLQDGCNNNLLTHTCTRGCSS

SEQ ID NO: 38 - RNA, 19 nt, Artificial Sequence
  siRNA oligonucleotide
gggaggaauucaucauauu

SEQ ID NO: 39 - RNA, 19 nt, Artificial Sequence
  siRNA oligonucleotide
gaagucaacuuugaccauu

SEQ ID NO: 40 - RNA, 19 nt, Artificial Sequence
  siRNA oligonucleotide
gcccuacauggcuccagaa

SEQ ID NO: 41 - DNA, 1302 bp, Homo sapiens
atggaggtggtggacccgcagcagctgggcatgttcacggagggcgagctgatgtcggtgggtatggacacgttcatccaccgcatcgactccaccgaggtcatctaccagccgcgccgcaagcgggccaagctcatcggcaagtacctgatgggggacctgctgggggaaggctcttacggcaaggtgaaggaggtgctggactcggagacgctgtgcaggagggccgtcaagatcctcaagaagaagaagttgcgaaggatccccaacggggaggccaacgtgaagaaggaaattcaactactgaggaggttacggcacaaaaatgtcatccagctggtggatgtgttatacaacgaagagaagcagaaaatgtatatggtgatggagtactgcgtgtgtggcatgcaggaaatgctggacagcgtgccggagaagcgtttcccagtgtgccaggcccacgggtacttctgtcagctgattgacggcctggagtacctgcatagccagggcattgtgcacaaggacatcaagccggggaacctgctgctcaccaccggtggcaccctcaaaatctccgacctgggcgtggccgaggcactgcacccgttcgcggcggacgacacctgccggaccagccagggctccccggctttccagccgcccgagattgccaacggcctggacaccttctccggcttcaaggtggacatctggtcggctggggtcaccctctacaacatcaccacgggtctgtaccccttcgaaggggacaacatctacaagttgtttgagaacatcgggaaggggagctacgccatcccgggcgactgtggccccccgctctctgacctgctgaaagggatgcttgagtacgaaccggccaagaggttctccatccggcagatccggcagcacagctggttccggaagaaacatcctccggctgaagcaccagtgcccatcccaccgagcccagacaccaaggaccggtggcgcagcatgactgtggtgccgtacttggaggacctgcacggcgcggacgaggacgaggacctcttcgacatcgaggatgacatcatctacactcaggacttcacggtgcccggacaggtcccagaagaggaggccagtcacaatggacagcgccggggcctccccaaggccgtgtgtatgaacggcacagaggcggcgcagctgagcaccaaatccagggcggagggccgggcccccaaccctgcccgcaaggcctgctccgccagcagcaagatccgccggctgtcggcctgcaagcagcagtga

SEQ ID NO: 42 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

SEQ ID NO: 43 - RNA, 19 nt, Artificial Sequence
  siRNA oligonucleotide
ccgucaagauccucaagaa

SEQ ID NO: 44 - RNA, 19 nt, Artificial Sequence
  siRNA oligonucleotide
gccaacgugaagaaggaaa

SEQ ID NO: 45 - RNA, 19 nt, Artificial Sequence
  siRNA oligonucleotide
cgcagcagcugggcauguu

SEQ ID NO: 46 - DNA, 2760 bp, Homo sapiens
atgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcgttgctgctctccaatccagcctaccgcctccttctggccacttacgcccgtccccctcgaggcccgggcccccccacacccgcctgggccaaacccaccaacacccaggcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccagcttctccttgttctccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggtcagctcaccgacgagcaggtcatcgagaacgcgggggagttcttccgggaccagggccggcagtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtga

SEQ ID NO: 47 - protein, 919 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNASFSLFSRNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGSAHRRAGHRERGGVLPGPGPAVYLSRPPACPQGLYELMLRCWSRESEQRPPFSQLHRFLAEDALNTV

SEQ ID NO: 48 - RNA, 19 nt, Artificial Sequence
  siRNA oligonucleotide
ggagcuaccggcugcguua

SEQ ID NO: 49 - RNA, 19 nt, Artificial Sequence
  siRNA oligonucleotide
gggauggacuccugucuua

SEQ ID NO: 50 - RNA, 19 nt, Artificial Sequence
  siRNA oligonucleotide
cagcuucuccaguggcuau

SEQ ID NO: 51 - DNA, 1158 bp, Homo sapiens
atggggtgcggcgccagcaggaaggtggtcccggggccaccagcgctggcttgggccaagcacgaaggtcaaaaccaagccggcgtcggaggcgcggggcctgggcccgaggcggcggcccaggcggcgcagaggatacaggtggctcgcttccgagccaagttcgacccccgggtccttgccaggtatgacatcaaagctcttattgggacaggcagtttcagcagggttgtcagggtagagcagaagaccaccaagaaaccttttgcaataaaagtgatggaaaccagagagagggaaggtagagaagcgtgcgtgtctgagctgagcgtcctgcggcgggttagccatcgttacattgtccagctcatggagatctttgagactgaggatcaagtttacatggtaatggagctggctaccggaggggagctctttgatcgactcattgctcagggatcctttacagagcgggatgccgtcaggatcctccagatggttgctgatgggattaggtatttgcatgcgctgcagataactcataggaatctaaagcctgaaaacctcttatactatcatccaggtgaagagtcgaaaattttaattacagattttggtttggcatactccgggaaaaaaagtggtgactggacaatgaagacactctgtgggaccccagagtacatagctcctgaggttttgctaaggaagccttataccagtgcagtggacatgtgggctcttggtgtgatcacatatgctttacttagcggattcctgccttttgatgatgaaagccagacaaggctttacaggaagattctgaaaggcaaatataattatacaggagagccttggccaagcatttcccacttggcgaaggactttatagacaaactactgattttggaggctggtcatcgcatgtcagctggccaggccctggaccatccctgggtgatcaccatggctgcagggtcttccatgaagaatctccagagggccatatcccgaaacctcatgcagagggcctctccccactctcagagtcctggatctgcacagtcttctaagtcacattattctcacaaatccaggcatatgtggagcaagagaaacttaaggatagtagaatcgccactgtctgcgcttttgtaa

SEQ ID NO: 52 - protein, 385 aa, Homo sapiens
MGCGASRKVVPGPPALAWAKHEGQNQAGVGGAGPGPEAAAQAAQRIQVARFRAKFDPRVLARYDIKALIGTGSFSRVVRVEQKTTKKPFAIKVMETREREGREACVSELSVLRRVSHRYIVQLMEIFETEDQVYMVMELATGGELFDRLIAQGSFTERDAVRILQMVADGIRYLHALQITHRNLKPENLLYYHPGEESKILITDFGLAYSGKKSGDWTMKTLCGTPEYIAPEVLLRKPYTSAVDMWALGVITYALLSGFLPFDDESQTRLYRKILKGKYNYTGEPWPSISHLAKDFIDKLLILEAGHRMSAGQALDHPWVITMAAGSSMKNLQRAISRNLMQRASPHSQSPGSAQSSKSHYSHKSRHMWSKRNLRIVESPLSALL

SEQ ID NO: 53 - RNA, 19 nt, Artificial Sequence
  siRNA oligonucleotide
ggaucaaguuuacauggua

SEQ ID NO: 54 - RNA, 19 nt, Artificial Sequence
  siRNA oligonucleotide
gacugaggaucaaguuuac

SEQ ID NO: 55 - RNA, 19 nt, Artificial Sequence
  siRNA oligonucleotide
gcggguuagccaucguuac

SEQ ID NO: 56 - DNA, 2079 bp, Homo sapiens
atggagaagtacgagcggatccgagtggtggggagaggtgccttcgggattgtgcacctgtgcctgcgaaaggctgaccagaagctggtgatcatcaagcagattccagtggaacagatgaccaaggaagagcggcaggcagcccagaatgagtgccaggtcctcaagctgctcaaccaccccaatgtcattgagtactacgagaacttcctggaagacaaagcccttatgatcgccatggaatatgcaccaggcggcactctggctgagttcatccaaaagcgctgtaattccctgctggaggaggagaccatcctgcacttcttcgtgcagatcctgcttgcactgcatcatgtgcacacccacctcatcctgcaccgagacctcaagacccagaacatcctgcttgacaaacaccgcatggtcgtcaagatcggtgatttcggcatctccaagatccttagcagcaagagcaaggcctacacggtggtgggtaccccatgctatatctcccctgagctgtgtgagggcaagccctacaaccagaagagtgacatctgggccctgggctgtgtcctctacgagctggccagcctcaagagggctttcgaggctgcgaacttgccagcactggtgctgaagatcatgagtggcacctttgcacctatctctgaccggtacagccctgagcttcgccagctggtcctgagtctactcagcctggagcctgcccagcggccaccactcagccacatcatggcacagcccctctgcatccgtgccctcctcaacctccacaccgacgtgggcagtgtccgcatgcggagggcagagaagtccgtggcccccagcaacacagggagcaggaccaccagtgtccgctgcagaggtatcccccggggacctgtgaggccagccatcccaccaccactgtcgtcagtgtatgcctggggtggtgggctgggcacccccctgcggctgccaatgctcaacacagaggtggtccaggtggcagctgggcgcacgcagaaagccggcgtcacgcgctctgggcgtctcatcctgtgggaggccccacccctaggtgcaggcggaggcagtctccttcctggggcagtggagcagccacagccccagttcatctcgcgtttcctggagggccagtcgggtgtgaccatcaagcacgtggcctgtggggacttcttcactgcctgcctgactgacagaggcatcatcatgacattcggcagcggcagcaatgggtgcctaggccatggcagcctcactgacatcagccagcccaccattgtggaggctttgctgggctatgaaatggtgcaggtggcctgtggggcctctcacgtgctggccctgtccactgagcgagaactatttgcctggggccgtggagacagcggcagactggggctaggcaccagggagtcccacagctgcccccagcaggtgcccatgcccccaggacaggaagctcagcgagttgtatgtggtatcgattcctccatgatcctcactgtgcctggccaagccctagcctgtgggagcaacaggttcaacaagctgggcctggaccacctctccctgggggaggagcctgtcccccaccagcaagtggaggaggccctgagcttcacactactaggctctgcacccctggaccaggagcctctgctgagtatagacctgggcactgctcactcagctgctgtgactgcctcgggtgattgctacacttttggcagcaatcagcacggacagttgggcaccaatactcgccgaggcagtcgggcaccctgtaaggtccaaggccttgagggcatcaagatggcaatggtagcctgtggggatgccttcactgtagctattggggcagagagcgaagtgtactcttggggcaaaggggcgcgaggtcgattgggaaggagggatgaggatgccggactccctcggccagtgcagttggatgagacacacccttacacggtgacttccgtgtcctgttgccatggaaacaccctcctggctgttcgatcggtcacagatgagccggtccccccctga

SEQ ID NO: 57 - protein, 692 aa, Homo sapiens
MEKYERIRVVGRGAFGIVHLCLRKADQKLVIIKQIPVEQMTKEERQAAQNECQVLKLLNHPNVIEYYENFLEDKALMIAMEYAPGGTLAEFIQKRCNSLLEEETILHFFVQILLALHHVHTHLILHRDLKTQNILLDKHRMVVKIGDFGISKILSSKSKAYTVVGTPCYISPELCEGKPYNQKSDIWALGCVLYELASLKRAFEAANLPALVLKIMSGTFAPISDRYSPELRQLVLSLLSLEPAQRPPLSHIMAQPLCIRALLNLHTDVGSVRMRRAEKSVAPSNTGSRTTSVRCRGIPRGPVRPAIPPPLSSVYAWGGGLGTPLRLPMLNTEVVQVAAGRTQKAGVTRSGRLILWEAPPLGAGGGSLLPGAVEQPQPQFISRFLEGQSGVTIKHVACGDFFTACLTDRGIIMTFGSGSNGCLGHGSLTDISQPTIVEALLGYEMVQVACGASHVLALSTERELFAWGRGDSGRLGLGTRESHSCPQQVPMPPGQEAQRVVCGIDSSMILTVPGQALACGSNRFNKLGLDHLSLGEEPVPHQQVEEALSFTLLGSAPLDQEPLLSIDLGTAHSAAVTASGDCYTFGSNQHGQLGTNTRRGSRAPCKVQGLEGIKMAMVACGDAFTVAIGAESEVYSWGKGARGRLGRRDEDAGLPRPVQLDETHPYTVTSVSCCHGNTLLAVRSVTDEPVPP

SEQ ID NO: 58 - RNA, 19 nt, Artificial Sequence
  siRNA oligonucleotide
ggcuuugcugggcuaugaa

SEQ ID NO: 59 - RNA, 19 nt, Artificial Sequence
  siRNA oligonucleotide
cccagaacauccugcuuga

SEQ ID NO: 60 - RNA, 19 nt, Artificial Sequence
  siRNA oligonucleotide
gcggcacucuggcugaguu